(19) United States
(12) Patent Application Publication
(10) Pub. No.: US 2019/0038762 A1
(43) Pub. Date: Feb. 7, 2019

(54) GCC-TARGETED ANTIBODY-DRUG CONJUGATES

(71) Applicants: MILLENNIUM PHARMACEUTICALS, INC., Cambridge, MA (US); IMMUNOGEN, INC., Waltham, MA (US)

(72) Inventors: Ole Petter VEIBY, Westborough, MA (US); Ravi V. J. CHARI, Newton, MA (US); John M. LAMBERT, Cambridge, MA (US); Katharine C. LAI, Littleton, MA (US); Robert W. HERBST, Braintree, MA (US); Scott A. HILDERBRAND, Swampscott, MA (US)

(73) Assignees: MILLENNIUM PHARMACEUTICALS, INC., Cambridge, MA (US); IMMUNOGEN, INC., Waltham, MA (US)

(21) Appl. No.: 16/075,023

(22) PCT Filed: Feb. 3, 2017
(86) PCT No.: PCT/US2017/016458

§ 371 Date: Aug. 2, 2018

Related U.S. Application Data
(60) Provisional application No. 62/292,087, filed on Feb. 5, 2016.

(51) Int. Cl. A61K 47/68; C07K 16/40; C07K 16/30; A61P 35/00

(52) U.S. Cl. CPC A61K 47/6803; C07K 16/40; C07K 16/30; A61P 35/00; A61K 47/6871; A61K 47/6863; A61K 47/6859

(57) ABSTRACT

This invention relates to antibody-drug conjugates capable of delivering cytotoxic compounds to cancers expressing the guanylyl cyclase C (GCC) transmembrane cell surface receptor.

The present application claims the benefit of priority to U.S. Provisional Patent Application No. 62/292,087, filed 5 Feb. 2016, the contents of which are hereby incorporated herein its entirety.

This invention relates to antibody-drug conjugates capable of delivering cytotoxic compounds to cancers expressing the guanylyl cyclase C (GCC) transmembrane cell surface receptor.

GCC functions in the maintenance of intestinal fluid, electrolyte homeostasis, and cell proliferation. Arshad and Visweswariah, FEBS Letters 586:2835-2840 (2012). In normal adult mammals, functional GCC is expressed by mucosal cells lining the small intestine, large intestine, and rectum. These cells undergo homeostatic cycles of proliferation, migration, differentiation, and apoptosis, and an imbalance between proliferation and apoptosis can lead to the formation of tumors within the gastrointestinal tract. Arshad and Visweswariah (2012).

GCC is a surface protein with anatomically compartmentalized expression allowing selective targeting to antigen-expressing tumors. GCC expression is maintained upon neoplastic transformation of intestinal epithelial cells, with expression in all primary and metastatic colorectal tumors. Carrithers et al., Proc. Natl. Acad. Sci. USA 93(25):14827-14832 (1996). GCC-targeting agents are not able to penetrate the intestinal wall and reach the site where GCC is normally found, but do reach cancer cells that continue to express GCC on the cell surface.

E. coli heat-stable enterotoxin, a ligand for GCC, has been described as a potential targeting vehicle for the delivery of anticancer therapeutic protein agents to colorectal cancer cells. Buc et al., Eur. J. Cancer 41(11):1618-1627 (2005). In addition, anti-GCC antibody-drug conjugates have previously been demonstrated to have activity against GCC in pancreatic cancer. Veiby, Abstract PR12/B19 presented at the International Conference on Molecular Targets and Cancer Therapeutics Oct. 19-23, 2013, Boston. However, not all antibody-drug conjugates will meet the biological profile necessary to be taken into the clinic.

It is not possible to predict in advance, simply based on an antibody profile, or a drug payload profile, which antibody-drug conjugates will be sufficiently safe and effective for clinical applications. For example, a particular drug payload may function perfectly well when conjugated to an antibody directed to one target, but it may not work nearly as well when conjugated to an antibody directed to a different target, or even to a different antibody directed to the same target. Why different antibody-drug conjugates display different anti-tumor activity in vivo is not sufficiently well understood to allow accurate predictions in the design of new antibody-drug conjugates. It is speculated that an unpredictable interplay of many factors play a role. These factors may include, for example, the binding affinity of an antibody-drug conjugate to a target antigen, the ability of the conjugate to penetrate solid tumors, as well as the half-life in circulation for proper exposure to tumors without causing toxicity.

The complexity and unpredictability is well demonstrated by antibody affinity alone. Antibodies or antibody-drug conjugates with high affinity track with better cellular uptake, which leads to a higher level of the cytotoxic payloads released inside the cells. Higher affinity is also known to enhance the antibody-dependent cellular cytotoxicity (ADCC). All these attributes favor the cell killing property of antibody-drug conjugates. However, it is also known that high affinity of an antibody or antibody-drug conjugate can prevent efficient tumor penetration via an “antigen barrier effect”, suggesting that in order to achieve a strong anti-tumor activity in vivo, affinity of the antibody-drug conjugate has to be just right: not too high or not too low. To date, it is not known how to predict what will be the most efficient or effective level of affinity for an antibody-drug conjugate.

In addition, in vivo anti-tumor activity cannot be predicted by the mechanism of linkers and payloads alone. For example, O. Ab et al, Mol. Cancer Ther. 14(&):1605-1613 (2015) demonstrated that, when tested in preclinical cancer models, the same antibody conjugated to the same anti-tubulin toxin via different linkers exhibited dramatically different anti-tumor activity. This example is particularly surprising because the chemical structures of the two linkers are very similar. Moreover, the linker present in the superior conjugate contained a hydrophilic moiety. Hydrophilic metabolites are generally less membrane-permeable, and are thought to be slower in efflux from the lysosomes (the site of conjugate degradation), leading to a delay in the anti-tubulin activity of the released payload. This finding argues for an “ideal” kinetics of payload delivery, but to date, there is no insight into what constitutes such kinetics. Adding to this complexity is the open question of whether ideal kinetics of payload delivery, even if defined for a particular cell type, would apply to all cell types. Thus, it is not possible to predict the most effective in vivo anti-tumor activity merely from the chemical composition of the linker or payload.

Further supporting the unpredictability of antibody-drug conjugate activity in vivo are two antibody-drug conjugates (both targeting liquid tumors), that share the same linker payload (SPDB-DM4) conjugated to different antibodies. The first, an anti-CD33-SPDB-DM4 conjugate, was found to be ineffective in vivo. S. Lapusan et al., Invest. New Drugs 30:1121-1131 (2012). In contrast, an anti-CD19-SPDB-DM4 conjugate has been shown to be effective against lymphoma in clinical trials. V. Ribrag, at al., Clin. Cancer Res. 20(1):213-220 (2014).

Consequently, it is not surprising that, to date, no drug products containing an anti-GCC antibody have been approved for cancer treatment, let alone an antibody-drug conjugate that can selectively deliver cytotoxic agents to cancer cells expressing the GCC antigen. Thus, an unmet need exists for antibody-drug conjugates that treat cancers expressing GCC.

The invention provides, in part, antibody-drug conjugates comprising an antibody molecule which comprises a heavy chain variable region (VH) comprising complementarity determining region (CDR) amino acid sequences of SEQ ID NO:1 (VHCDR1), SEQ ID NO:2 (VHCDR2), and SEQ ID NO:3 (VHCDR3) and a light chain variable region (VL) comprising CDR amino acid sequences of SEQ ID NO:4 (VLCDR1), SEQ ID NO:5 (VLCDR2), and SEQ ID NO:6 (VLCDR3), conjugated to a cytotoxic drug agent (CDA) selected from

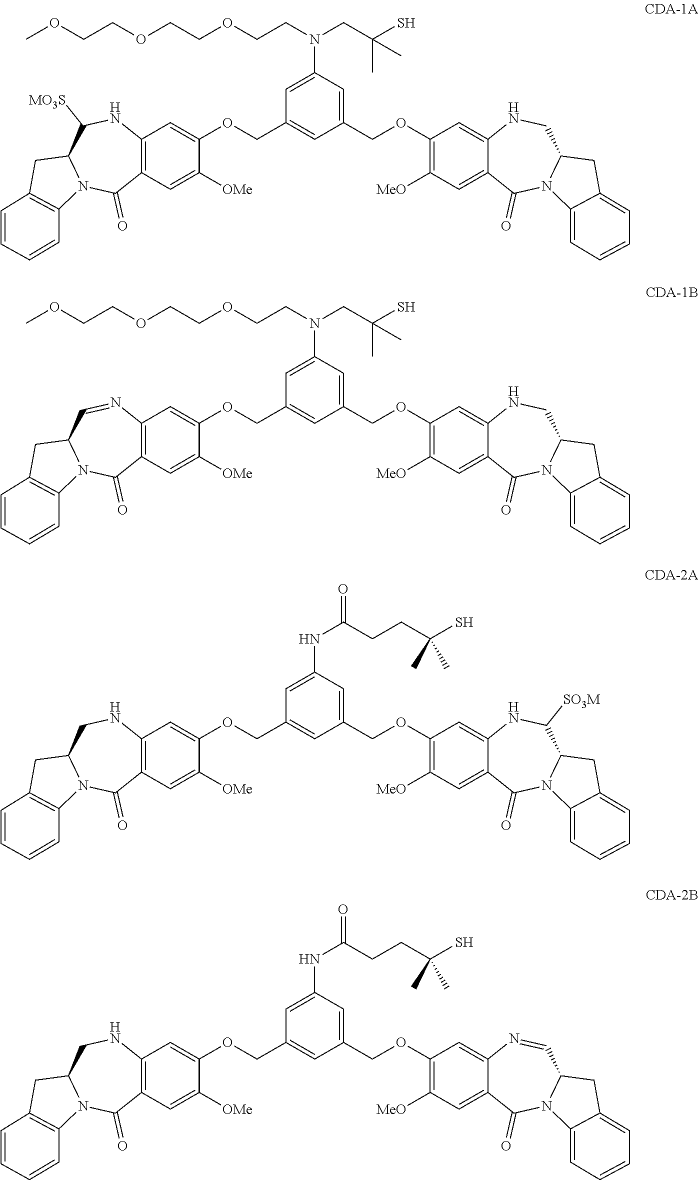

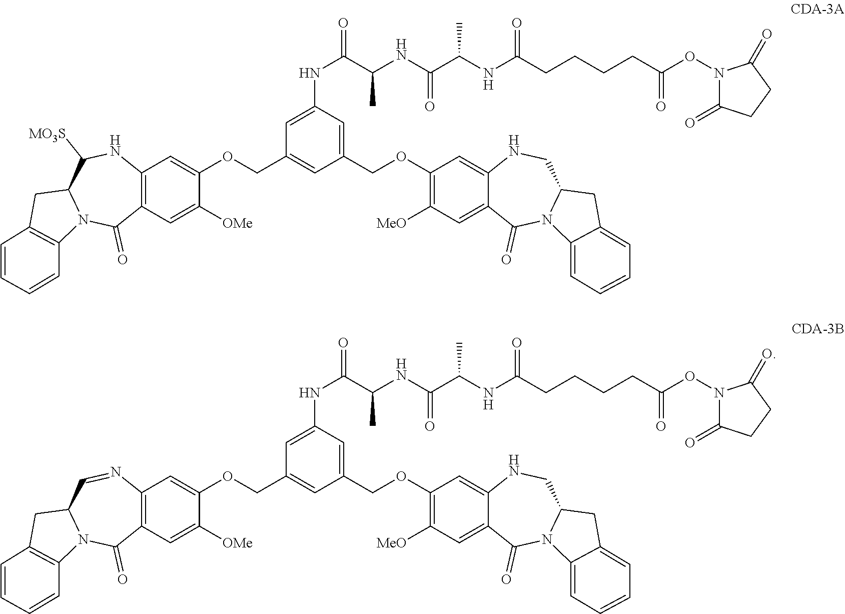

The antibody molecule may be linked to a CDA through any suitable linker, such as, e.g., N-succinimidyl-3-(2-pyridyldithio)propionate (SPDP) or N-succinimidyl-4-(2-pyridyldithio)-2-sulfo butanoate (sulfo-SPDB).

In some embodiments, the VH of the antibody molecule comprises the amino acid sequence of SEQ ID NO:7, or a sequence that is at least 85% identical to SEQ ID NO:7, and the VL comprises the amino acid sequence of SEQ ID NO:8 or a sequence that is at least 95% identical to SEQ ID NO:8. In some embodiments, the antibody molecule comprises a heavy chain comprising the amino acid sequence of SEQ ID NO:9 or a sequence that is at least 95% identical to SEQ ID NO:9 and a light chain comprising the amino acid sequence of SEQ ID NO:10 or a sequence that is at least 95% identical to SEQ ID NO:10.

Additional aspects of the invention include methods of targeting anticancer therapy to tumor cells expressing GCC antigen, methods of inhibiting the growth of a tumor by administering an antibody-drug conjugate of the invention, methods of reducing the size of a tumor by administering an antibody-drug conjugate of the invention, and methods of treating a cancer characterized by the expression of GCC by administering an antibody-drug conjugate of the invention. In some embodiments, the tumor/cancer to be treated is a cancer of the gastrointestinal system (e.g., colorectal cancer, esophageal cancer, or stomach cancer). In some embodiments, the tumor/cancer to be treated is pancreatic cancer.

### BRIEF DESCRIPTION OF THE FIGURES

FIG. 1A-FIG. 1D show cell binding data to GCC-expressing cells. FIG. 1A reflects affinity values for unconjugated 5F9 antibody. FIG. 1B, FIG. 1C, and FIG. 1D reflect affinity values for antibody-drug conjugates 5F9-CDA-1, 5F9-CDA-2, and 5F9-CDA-3, respectively.

FIG. 2A-FIG. 2C depict the relative potency of 5F9-CDA conjugates on HEK293-GCC#2 cells.

FIG. 3A-FIG. 3C demonstrate in vivo efficacy of 5F9-CDA-1, 5F9-CDA-2, and 5F9-CDA-3, respectively, in HEK293-GCC#2 tumor-bearing mice.

FIG. 4A-FIG. 4C demonstrate in vivo efficacy of 5F9-CDA-1, 5F9-CDA-2, and 5F9-CDA-3, respectively, in a primary human tumor xenograft model for colorectal cancer, PHTX(a) tumor-bearing mice after a single dose. FIG. 4D-FIG. 4F demonstrate in vivo efficacy of 5F9-CDA-1, 5F9-CDA-2, and 5F9-CDA-3, respectively, in a primary human tumor xenograft model for colorectal cancer, PHTX(a) tumor-bearing mice after fractionated doses.

FIG. 5A-FIG. 5C demonstrate in vivo efficacy of 5F9-CDA-1, 5F9-CDA-2, and 5F9-CDA-3, respectively, in a primary human tumor xenograft model for colorectal cancer, PHTX(b) tumor-bearing mice.

FIG. 6A-FIG. 6B demonstrate in vivo efficacy of 5F9-CDA-2, and 5F9-CDA-3, respectively, in a primary human tumor xenograft model for colorectal cancer, PHTX(c) tumor-bearing mice.

FIG. 7A-FIG. 7C depict the pharmacokinetic (PK) profiles of HEK293-GCC tumor-bearing mice following administration of 5F9-CDA-1, 5F9-CDA-2, or 5F9-CDA-3 as described in Example 8.

FIGS. 8A and 8B depict the pharmacodynamic (PD) profiles of HEK293-GCC tumor-bearing mice following administration of 5F9-CDA-1, 5F9-CDA-2, or 5F9-CDA-3 as described in Example 8.

FIG. 9A depicts the liquid chromatogram of the sulfonation reaction. FIG. 9 B depicts the mass spectrometry profile the peak corresponding to CDA-3B.

### DESCRIPTION OF EMBODIMENTS

Unless otherwise defined herein, scientific and technical terms used in connection with the present invention have the meanings that are commonly understood by those of ordinary skill in the art. Generally, nomenclature utilized in connection with, and techniques of, cell and tissue culture, molecular biology, and protein and oligo- or polynucleotide chemistry and hybridization described herein are those known in the art.

### Antibody Molecules

The term “antibody molecule,” as used herein, refers to an antibody or an antigen binding fragment thereof comprising SEQ ID NOs 1-6. Antibody molecules include single chain antibody molecules (see, e.g., scFv, see. e.g., Bird et al. Science 242:423-426 (1988) and Huston et al. Proc. Natl. Acad. Sci. USA 85:5879-5883 (1988)), and single domain antibody molecules (see, e.g., W09404678). “Antibody molecule” may also refer to two-chain and multi-chain immunoglobulin proteins and glycoproteins. As used herein, the term “antibody fragment” or “antigen binding fragment” of an antibody refers, e.g., to Fab, Fab′, F(ab′)2, and Fv fragments, single chain antibodies, functional heavy chain antibodies (nanobodies), as well as any portion of an antibody having specificity for GCC. Antigen binding fragments can be produced by recombinant techniques, or by enzymatic or chemical cleavage of an intact antibody. The term, antigen binding fragment, when used with a single chain, e.g., a heavy chain, of an antibody having a light and heavy chain means that the fragment of the chain is sufficient such that when paired with a complete variable region of the other chain, e.g., the light chain, it will allow binding of at least 25%, 50%, 75%, 85%, or 90% of that seen with the whole heavy and light variable region.

The term “antibody molecule” also includes synthetic and genetically engineered variants. In some embodiments, the variants comprise CDR sequences of SEQ ID NOs 1-6 and VH and VL sequences that are at least 95% identical to SEQ ID NO:7 and SEQ ID NO:8, respectively. In some embodiments, the variants comprise CDR sequences of SEQ ID NOs 1-6 and heavy and light chain sequences that are at least 95% identical to SEQ ID NO:9 and SEQ ID NO:10, respectively. In some embodiments, the antibody molecules comprise the CDR sequences of SEQ ID NOs 1-6, wherein 1, 2, 3, 4, or 5 conservative amino acid substitutions have been made in one or more of the CDR sequences. In some embodiments, the antibody molecules comprise the CDR sequences of SEQ ID Nos 1-6, wherein 1, 2, 3, 4, or 5 non-conservative amino acid substitutions have been made in one or more of the CDR sequences. These amino acid substitutions may be accompanied by either increase or decrease in the affinity, avidity, on-rate (Kₒₙ), or off-rate (K_{off}) of the antibody that provides beneficial properties to the antibody, such as, e.g., better tumor penetration, higher accumulation in tumor, a change in antibody-dependent cellular cytotoxicity (ADCC), better efficacy, better toxicity profiles, or wider therapeutic window. See, e.g., the effect of affinity on the uptake and penetration of an antibody in solid tumors described in Rudnick et al. Cancer Res. 71(6): 2250-2259 (2011). In some embodiments, the antibody molecule comprises SEQ ID NO:9 and SEQ ID NO:10, wherein one or both sequences have been modified in the constant domain to improve stability, reduce immunogenicity, or provide other beneficial properties to the antibody, such as, e.g., altered effector functions. See, e.g., modifications to constant domain sequences described in Kubota et al. Cancer Sci. 100(9):1566-1572 (2009), US 2006/0275282, and U.S. Pat. No. 9,085,625.

In certain embodiments, the antibody molecules employed in the antibody-drug conjugates of the invention comprise human constant regions. Sequences of human constant region genes may be found in Kabat et al. Sequences of Proteins of Immunological Interest, N.I.H. Publication No. 91-3242 (1991). Human constant region genes are also readily available from known clones. The choice of isotype will be guided by the desired effector functions, such as complement fixation, or activity in antibody-dependent cellular cytotoxicity. Isotypes can be IgG1, IgG2, IgG3, or IgG4. In particular embodiments, antibody molecules of the invention are IgG1 and IgG2. Either of the human light chain constant regions, kappa or lambda, may be used. The chimeric, humanized antibody is then expressed by conventional methods.

In some embodiments, an anti-GCC antibody molecule of the invention can draw ADCC to a cell expressing GCC, e.g., a tumor cell. Antibodies with the IgG1 and IgG3 isotypes are useful for eliciting effector function in an antibody-dependent cytotoxic capacity, due to their ability to bind the Fc receptor. Antibodies with the IgG2 and IgG4 isotypes are useful to minimize an ADCC response because of their low ability to bind the Fc receptor. In related embodiments substitutions in the Fc region or changes in the glycosylation composition of an antibody, e.g., by growth in a modified eukaryotic cell line, can be made to enhance the ability of Fc receptors to recognize, bind, and/or mediate cytotoxicity of cells to which anti-GCC antibodies bind. See, e.g., U.S. Pat. Nos. 7,317,091; 5,624,821; and publications including WO 00/42072, Shields, et al. J. Biol. Chem. 276:6591-6604 (2001), Lazar et al. Proc. Natl. Acad. Sci. USA 103:4005-4010 (2006), Satoh et al. Expert Opin. Biol. Ther. 6:1161-1173 (2006). In certain embodiments, the antibody or antigen-binding fragment (e.g., antibody of human origin, human antibody) can include amino acid substitutions or replacements that alter or tailor function (e.g., effector function). For example, a constant region of human origin (e.g., γ1 constant region, γ2 constant region) can be designed to reduce complement activation and/or Fc receptor binding. (See, for example, U.S. Pat. Nos. 5,648,260; 5,624,821; and 5,834,597, the entire teachings of which are incorporated herein by reference.) Preferably, the amino acid sequence of a constant region of human origin that contains such amino acid substitutions or replacements is at least about 95% identical over the full length to the amino acid sequence of the unaltered constant region of human origin, more preferably at least about 99% identical over the full length to the amino acid sequence of the unaltered constant region of human origin.

In still another embodiment, effector functions can also be altered by modulating the glycosylation pattern of the antibody. By altering is meant deleting one or more carbohydrate moieties found in the antibody, and/or adding one or more glycosylation sites that are not present in the antibody. For example, antibodies with enhanced ADCC activities with a mature carbohydrate structure that lacks fucose attached to an Fc region of the antibody are described in US 2003/0157108. See also US 2004/0093621.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished, for example, by expressing the antibody in a host cell with altered glycosylation machinery. Cells with altered glycosylation machinery have been described in the art and can be used as host cells in which are engineered to express recombinant antibodies of the invention to thereby produce an antibody with altered glycosylation. For example, EP 1,176,195 describes a cell line with a functionally disrupted FUT8 gene, which encodes a fucosyl transferase, such that antibodies expressed in such a cell line exhibit hypofucosylation. WO 03/035835 describes a variant CHO cell line, Lec13 cells, with reduced ability to attach fucose to Asn(297)-linked carbohydrates, also resulting in hypofucosylation of antibodies expressed in that host cell. See also Shields, R. L. et al., J. Biol. Chem. 277:26733-26740 (2002). WO 99/54342 describes cell lines engineered to express glycoprotein-modifying glycosyl transferases (e.g., beta(1,4)-N acetylglucosaminyl-transferase III (GnTIII)) such that antibodies expressed in the engineered cell lines exhibit increased bisecting GlcNac structures which results in increased ADCC activity of the antibodies. See also Umana et al., Nat. Biotech. 17:176-180 (1999).

In certain embodiments, the antibody molecule may be a bispecific, biparatopic, or bifunctional antibody, wherein at least one pair of binding sequences comprises the CDR sequences of SEQ ID NOs 1-6. In some embodiments, both binding sites of a bispecific or bifunctional antibody comprise the CDR sequences of SEQ ID NOs 1-6. In some embodiments, the bispecific or bifunctional antibody comprises the amino acid sequences of SEQ ID NOs 7 and 8 or a variant thereof that comprises sequences that are at least 95% identical to SEQ ID NO:7 and/or SEQ ID NO:8.

Preferred antibody molecules for use in the antibody-drug conjugates of the invention are fully human antibody molecules described in WO 2011/050242, incorporated herein by reference for its disclosure of antibody molecule 5F9 and variants thereof as well as recombinant methods of making such antibody molecules. Human mAb5F9 (IgG2, kappa) can be produced by hybridoma 46.5F9.8.2, deposited on Jan. 10, 2007 at American Type Culture Collection (ATCC) under Accession No. PTA-8132. However, other methods of making antibodies are well-known in the art. For example, antibody molecules may be produced in transgenic mice generated by XENOMOUSE™ technology described in U.S. Pat. Nos. 6,162,963; 6,150,584; 6,114,598; and 6,075,181. Other antibody producing transgenic mice may be made using a minilocus approach, such as that described in U.S. Pat. Nos. 5,545,807; 5,545,806; and 5,625,825. Additional antibody producing mice include the HUMAB-MOUSE™, the KIRIN TC MOUSE™, and KM-MOUSE®.

Alternatively, antibody molecules may be expressed in cultured cells. More specifically, sequences encoding particular antibodies can be cloned from cells producing the antibodies and used for transformation of a suitable mammalian host cell. In some embodiments, spleen and/or lymph node lymphocytes from immunized mice are isolated from the mice and plated in plaque assays as described previously in Babcook et al., Proc. Nat. Acad. Sci. USA 93:7843-7848 (1996). Briefly, cells are plated in agar with sheep red blood cells, coated with GCC antigen, and cells secreting mAb against the GCC antigen would fix complement and lyse the red blood cells immediately surrounding the mAb-producing cells. Cells within the cleared plaques are lifted for sequencing of the immunoglobulin sequences and subcloning into expression vectors. Supernatants from transiently transfected cells containing GCC-specific mAb are subsequently screened by ELISA and for binding to cells by flow cytometry. The variable sequences, or a portion thereof of the produced human antibodies comprising CDRs which bind particular epitopes may be utilized for production of modified antibodies. For example, the variable regions of the produced antibodies may be spliced into an expression cassette for ease of transfer of constructs, increased expression of constructs, and/or incorporation of constructs into vectors capable of expression of full length antibodies or fragments thereof as described, e.g., in US 20060147445. Human antibodies may also be generated using in vitro activated B cells as described in U.S. Pat. Nos. 5,567,601 and 5,229,275.

In some embodiments, the expression cassette comprises the heavy chain constant region of an IgG isotype. The sequences of human constant region genes may be found in Kabat et al. (1991) Sequences of Proteins of Immunological Interest, N.I.H. Publication No. 91-3242. Human constant region genes are readily available from known clones. The choice of isotype will be guided by the desired effector functions, such as complement fixation, or activity in antibody-dependent cellular cytotoxicity. Isotypes can be IgG1, IgG2, IgG3, or IgG4. In particular embodiments, antibody molecules of the invention are IgG1 and IgG2. In more particular embodiments, the isotype is IgG1. Either of the human light chain constant regions, kappa or lambda, may be used.

The antibody molecules employed in the antibody-drug conjugates of the invention target and bind specifically to the extracellular domain of GCC. As used herein, “specific binding,” “bind(s) specifically” or “binding specificity” means, for an anti-GCC antibody molecule, that the antibody molecule binds to GCC, e.g., human GCC protein, with greater affinity than it does to a non-GCC protein, e.g., BSA. Typically an anti-GCC molecule will have a K_{d} for the non-GCC protein, e.g., BSA, which is greater than 2 times, greater than 10 times, greater than 100 times, greater than 1,000 times, greater than 10⁴ times, greater than 10⁵ times, or greater than 10⁶ times its K_{d} for GCC, e.g., human GCC protein. Determination of K_{d}, for GCC and for the non-GCC protein, e.g., BSA, should be performed under the same conditions.

Calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 30%, 40%, or 50%, at least 60%, or at least 70%, 80%, 90%, 95%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent homology between two sequences can be accomplished using a mathematical algorithm. The percent homology between two amino acid sequences can be determined using any method known in the art. For example, the algorithm described in Needleman and Wunsch, J. Mol. Biol. 48:444-453 (1970), which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. The percent homology between two nucleotide sequences can also be determined using the GAP program in the GCG software package (Accelerys, Inc. San Diego, Calif.), using an NWSgapdna.CMP matrix and a gap weight of 40, 50, 60, 70, or 80 and a length weight of 1, 2, 3, 4, 5, or 6. An exemplary set of parameters for determination of homology are a Blossum 62 scoring matrix with a gap penalty of 12, a gap extend penalty of 4, and a frameshift gap penalty of 5.

It is understood that the antibodies and antigen binding fragment thereof of the invention may have additional conservative or non-essential amino acid substitutions, which do not have a substantial effect on the polypeptide functions. It is also understood that the antibodies and antigen binding fragment thereof of the invention may have additional non-conservative amino acid substitutions, which do not have a substantial effect on the polypeptide functions. Whether or not a particular substitution will be tolerated, i.e., will not adversely affect desired biological properties, such as binding activity, can be determined as described in Bowie et al., Science 247:1306-1310 (1990) or Padlan et al., FASEB J. 9:133-139 (1995). A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). A “non-conservative amino acid substitution” is one in which the amino acid residue is replaced with any other amino acid.

A “non-essential” amino acid residue is a residue that can be altered from the wild-type sequence of the binding agent, e.g., the antibody, without abolishing or, without substantially altering a biological activity.

The antibody molecule in the antibody-drug conjugate of the invention draws the CDA to the cancer cell expressing GCC. Amino acid and nucleic acid sequences of exemplary antibody molecules of the invention are set forth in Table 1.

TABLE 1

| | | |
|---|---|---|
| VHCDR1 | SEQ ID | GYYWS |
| | NO: 1 | |
| VHCDR2 | SEQ ID | EINHRGNTNDNPSLKS |
| | NO: 2 | |
| VHCDR3 | SEQ ID | ERGYTYGNFDH |
| | NO: 3 | |
| VLCDR1 | SEQ ID | RASQSVSRNLA |
| | NO: 4 | |
| VLCDR2 | SEQ ID | GASTRAT |
| | NO: 5 | |
| VLCDR3 | SEQ ID | QQYKTWPRT |
| | NO: 6 | |
| 5F9 VH | SEQ ID | QVQLQQWGAGLLKPSETLSLTCAVFGGSFSGYYWS |
| | NO: 7 | WIRQPPGKGLEWIGEINHRGNTNDNPSLKSRVTIS |
| | | VDTSKNQFALKLSSVTAADTAVYYCARERGYTYGN |
| | | FDHWGQGTLVTVSS |
| 5F9 VL | SEQ ID | EIVMTQSPATLSVSPGERATLSCRASQSVSRNLAW |
| | NO: 8 | YQQKPGQAPRLLIYGASTRATGIPARFSGSGSGTE |
| | | FTLTIGSLQSEDFAVYYCQQYKTWPRTFGQGTNVE |
| | | IK |
| 5F9/ | SEQ ID | MGWSCIILFLVATATGVHSQVQLQQWGAGLLKPSE |
| hIgG1 | NO: 9 | TLSLTCAVFGGSFSGYYWSWIRQPPGKGLEWIGEI |
| heavy | | NHRGNTNDNPSLKSRVTISVDTSKNQFALKLSSVT |
| chain | | AADTAVYYCARERGYTYGNFDHWGQGTLVTVSSAS |
| | | TKGPSVFPLAPSSKSTSGGTAALGCLVKDYFPEPV |
| | | TVSWNSGALTSGVHTFPAVLQSSGLYSLSSVVTVP |
| | | SSSLGTQTYICNVNHKPSNTKVDKKVEPKSCDKTH |
| | | TCPPCPAPELLGGPSVFLFPPKPKDTLMISRTPEV |
| | | TCVVVDVSHEDPEVKFNWYVDGVEVHNAKTKPREE |
| | | QYNSTYRVVSVLTVLHQDWLNGKEYKCKVSNKALP |
| | | APIEKTISKAKGQPREPQVYTLPPSRDELTKNQVS |
| | | LTCLVKGFYPSDIAVEWESNGQPENNYKTTPPVLD |
| | | SDGSFFLYSKLTVDKSRWQQGNVFSCSVMHEALHN |
| | | HYTQKSLSLSPGK |
| 5F9/ | SEQ ID | MGWSCIILFLVATATGVHSEIVMTQSPATLSVSPG |
| hKappa | NO: 10 | ERATLSCRASQSVSRNLAWYQQKPGQAPRLLIYGA |
| light | | STRATGIPARFSGSGSGTEFTLTIGSLQSEDFAVY |
| chain | | YCQQYKTWPRTFGQGTNVEIKRTVAAPSVFIFPPS |
| | | DEQLKSGTASVVCLLNNFYPREAKVQWKVDNALQS |
| | | GNSQESVTEQDSKDSTYSLSSTLTLSKADYEKHKV |
| | | YACEVTHQGLSSPVTKSFNRGEC |
| 5F9/ | SEQ ID | GAATTCCTCACCATGGGATGGAGCTGTATCATCCT |
| hIgG1 | NO: 11 | CTTCTTGGTAGCAACAGCTACAGGTGTCCACTCCC |
| heavy | | AGGTGCAGCTACAGCAGTGGGGCGCAGGACTGTTG |
| chain | | AAGCCTTCGGAGACCCTGTCCCTCACCTGCGCTGT |
| nucleic | | CTTTGGTGGGTCTTTCAGTGGTTACTACTGGAGCT |
| acid | | GGATCCGCCAGCCCCCAGGGAAGGGGCTGGAGTGG |
| | | ATTGGGGAAATCAATCATCGTGGAAACACCAACGA |
| | | CAACCCGTCCCTCAAGAGTCGAGTCACCATATCAG |
| | | TAGACACGTCCAAGAACCAGTTCGCCCTGAAGCTG |
| | | AGTTCTGTGACCGCCGCGGACACGGCTGTTTATTA |
| | | CTGTGCGAGAGAACGTGGATACACCTATGGTAACT |
| | | TTGACCACTGGGGCCAGGGAACCCTGGTCACCGTC |
| | | AGCTCAGCCTCCACCAAGGGCCCATCGGTCTTCCC |
| | | CCTGGCACCCTCCTCCAAGAGCACCTCTGGGGGCA |
| | | CAGCGGCCCTGGGCTGCCTGGTCAAGGACTACTTC |
| | | CCCGAACCGGTGACGGTGTCGTGGAACTCAGGCGC |
| | | CCTGACCAGCGGCGTGCACACCTTCCCGGCTGTCC |
| | | TACAGTCCTCAGGACTCTACTCCCTCAGCAGCGTG |
| | | GTGACCGTGCCCTCCAGCAGCTTGGGCACCCAGAC |
| | | CTACATCTGCAACGTGAATCACAAGCCCAGCAACA |
| | | CCAAGGTGGACAAGAAAGTTGAGCCCAAATCTTGT |
| | | GACAAAACTCACACATGCCCACCGTGCCCAGCACC |
| | | TGAACTCCTGGGGGGACCGTCAGTCTTCCTCTTCC |
| | | CCCCAAAACCCAAGGACACCCTCATGATCTCCCGG |
| | | ACCCCTGAGGTCACATGCGTGGTGGTGGACGTGAG |
| | | CCACGAAGACCCTGAGGTCAAGTTCAACTGGTACG |
| | | TGGACGGCGTGGAGGTGCATAATGCCAAGACAAAG |
| | | CCGCGGGAGGAGCAGTACAACAGCACGTACCGTGT |
| | | GGTCAGCGTCCTCACCGTCCTGCACCAGGACTGGC |
| | | TGAATGGCAAGGAGTACAAGTGCAAGGTCTCCAAC |
| | | AAAGCCCTCCCAGCCCCCATCGAGAAAACCATCTC |
| | | CAAAGCCAAAGGGCAGCCCCGAGAACCACAGGTGT |
| | | ACACCCTGCCCCCATCCCGGGATGAGCTGACCAAG |
| | | AACCAGGTCAGCCTGACCTGCCTGGTCAAAGGCTT |
| | | CTATCCCAGCGACATCGCCGTGGAGTGGGAGAGCA |
| | | ATGGGCAGCCGGAGAACAACTACAAGACCACGCCT |
| | | CCCGTGCTGGACTCCGACGGCTCCTTCTTCCTCTA |
| | | CAGCAAGCTCACCGTGGACAAGAGCAGGTGGCAGC |
| | | AGGGGAACGTCTTCTCATGCTCCGTGATGCATGAG |
| | | GCTCTGCACAACCACTACACGCAGAAGAGCCTCTC |
| | | CCTGTCTCCGGGTAAATAATAGGGATAACAGGGTA |
| | | ATACTAGAG |
| 5F9/ | SEQ ID | GCGGCCGCCTCACCATGGGATGGAGCTGTATCATC |
| hKappa | NO: 12 | CTCTTCTTGGTAGCAACAGCTACAGGTGTCCACTC |
| light | | CGAAATAGTGATGACGCAGTCTCCAGCCACCCTGT |
| chain | | CTGTGTCTCCAGGGGAAAGAGCCACCCTCTCCTGC |
| nucleic | | AGGGCCAGTCAGAGTGTTAGCAGAAACTTAGCCTG |
| acid | | GTATCAGCAGAAACCTGGCCAGGCTCCCAGGCTCC |
| | | TCATCTATGGTGCATCCACCAGGGCCACTGGAATC |
| | | CCAGCCAGGTTCAGTGGCAGTGGGTCTGGGACAGA |
| | | GTTCACTCTCACCATCGGCAGCCTGCAGTCTGAAG |
| | | ATTTTGCAGTTTATTACTGTCAGCAGTATAAAACC |
| | | TGGCCTCGGACGTTCGGCCAAGGGACCAACGTGGA |
| | | AATCAAACGTACGGTGGCTGCACCATCTGTCTTCA |
| | | TCTTCCCGCCATCTGATGAGCAGTTGAAATCTGGA |
| | | ACTGCCTCTGTTGTGTGCCTGCTGAATAACTTCTA |
| | | TCCCAGAGAGGCCAAAGTACAGTGGAAGGTGGATA |
| | | ACGCCCTCCAATCGGGTAACTCCCAGGAGAGTGTC |
| | | ACAGAGCAGGACAGCAAGGACAGCACCTACAGCCT |
| | | CAGCAGCACCCTGACCCTGAGCAAAGCAGACTACG |
| | | AGAAACACAAAGTCTACGCCTGCGAAGTCACCCAT |
| | | CAGGGCCTGAGCTCGCCCGTCACAAAGAGCTTCAA |
| | | CAGGGGAGAGTGTTAGTCTAGA |

### Cytotoxic Drug Agents (CDAs)

Indolinobenzodiazepine derivatives employed in the antibody-drug conjugates of the invention have been described as having high potency and/or high therapeutic index (ratio of maximum tolerated dose to minimum effective dose) in vivo. The benzodiazepine derivative CDA-1 is described in U.S. Pat. No. 8,765,740, which is incorporated herein by reference for disclosure related to CDA-1. CDA-1 exists in sulfonated (CDA-1A) and unsulfonated (CDA-1B) forms:

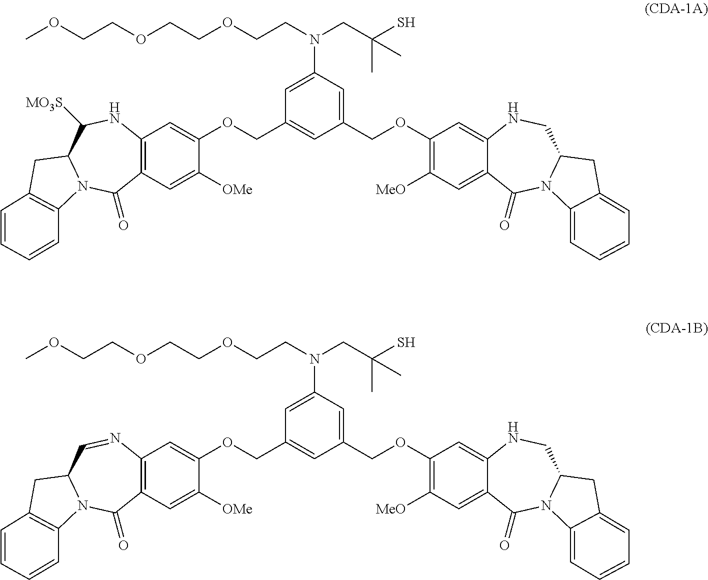

wherein M is —H or a pharmaceutically acceptable cation, such as,
e.g. Na⁺ or K⁺. Either CDA-1A or CDA-1B may be in the form of
any pharmaceutically acceptable salt.

CDA-2 is described in PCT/US2015/048064, incorporated herein by reference for disclosure related to CDA-2. Like CDA-1, CDA-2 exists in sulfonated (CDA-2A) and un-sulfonated (CDA-2B) forms:

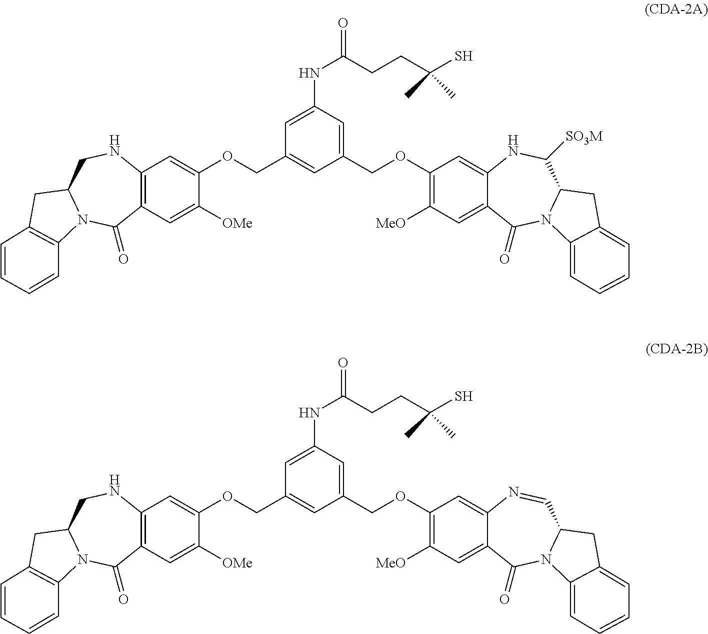

wherein M is —H or a pharmaceutically acceptable cation, such as, e.g. Na⁺ or K⁺. Either CDA-2A or CDA-2B may be in the form of any pharmaceutically acceptable salt.

CDA-3 is described in PCT/US2015/048059, which is incorporated herein by reference for disclosure related to CDA-3. CDA-3 exists in sulfonated (CDA-3A) and un-sulfonated (CDA-3B) forms:

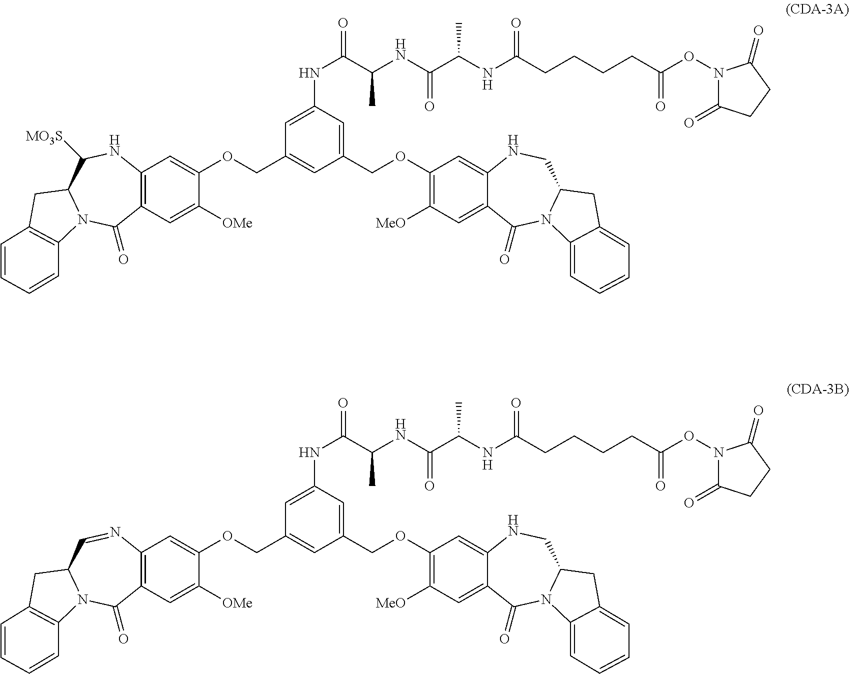

wherein M is —H or a pharmaceutically acceptable cation, such as, e.g. Na⁺ or K⁺. Either CDA-3A or CDA-3B may be in the form of any pharmaceutically acceptable salt.

The term “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion, or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum, and lithium.

### Antibody-Drug Conjugates

Antibody-drug conjugates are complex molecules combining both the antibody as the antigen target moiety and the drug or payload as the cell-killing or cytotoxic agent to selectively be delivered to the antigen-expressing cells (e.g., antigen-expressing tumor cells). The properties (e.g., efficacy or safety) of these type of molecules often cannot be predicted simply by conjugating the antibody having affinity to selected antigen target with a cytotoxic agent. Criteria for a successful antibody-drug conjugate include target antigen binding and internalization properties, cytotoxic activities, in vivo efficacy, PK/PD profiles, as well as safety and toxicity issues associated with using such antibody-drug conjugates. As shown below in the working examples, the antibody-drug conjugates of this invention each exhibited desirable properties.

The antibody molecules employed in the antibody-drug conjugates of the invention may be conjugated to the cytotoxic drug agent (CDA-1, CDA-2, or CDA-3) by any suitable method, or as disclosed in Example 5 herein, to produce the following antibody-drug conjugates:

or a pharmaceutically acceptable salt thereof, wherein M is —H or a pharmaceutically acceptable cation, such as, e.g. N⁺ or K⁺ and wherein HN [symbol] is an antibody comprising a heavy chain amino acid sequence of SEQ ID NO:9 and a light chain amino acid sequence of SEQ ID NO:10. The NH group attached to the antibody refers to the amino group side chain of a lysine residue of such antibody.

The terms “antibody-drug conjugate,” “antibody conjugate,” “immunoconjugate,” “conjugate,” and “ADC” are used interchangeably and refer to an antibody that is conjugated to a non-antibody moiety, e.g., a cytotoxic drug agent. The terms “linker,” “linker moiety,” or “linking group,” as defined herein, refer to a moiety that connects two groups, such as an antibody and a cytotoxic compound, together. In some embodiments, the antibody-drug conjugates of the invention comprise a cytotoxic drug agent (CDA-1, CDA-2, or CDA-3) and an antibody, wherein the cytotoxic drug agent is covalently linked to the antibody. In certain embodiments, the antibody-drug conjugates of the invention comprise a cytotoxic drug agent (CDA-1 or CDA-2) and an antibody, wherein the cytotoxic drug agent is covalently linked to the antibody through a linker (e.g., sulfo-SPDB). In other embodiments, the cytotoxic drug agent (CDA-3) has a reactive group (e.g., N-hydroxysuccinimide ester) that can directly form a covalent bond with the antibody.

A variety of suitable linkers (e.g., heterobifunctional reagents for connecting an antibody molecule to a cytotoxic drug agent) are known in the art. The linker can be cleavable, e.g., under physiological conditions, e.g., under intracellular conditions, such that cleavage of the linker releases the drug in the intracellular environment. In other embodiments, the linker is not cleavable, and the drug is released, for example, by antibody degradation.

The linker can be bonded to a chemically reactive group on the antibody moiety, e.g., to a free amino, imino, hydroxyl, thiol, or carboxyl group (e.g., to the N- or C-terminus, to the epsilon amino group of one or more lysine residues, to the free carboxylic acid group of one or more glutamic acid or aspartic acid residues, to the sulfhydryl group of one or more cysteinyl residues, or to the hydroxyl group of one or more serine or threonine residues). The site to which the linker is bound can be a natural residue in the amino acid sequence of the antibody moiety, or it can be introduced into the antibody moiety, e.g., by DNA recombinant technology (e.g., by introducing a cysteine or protease cleavage site in the amino acid sequence) or by protein biochemistry (e.g., reduction, pH adjustment, or proteolysis).

Typically, the linker is substantially inert under conditions for which the two groups it is connecting are linked. The term “bifunctional crosslinking agent,” “bifunctional linker” or “crosslinking agent” refers to a modifying agent that possess two reactive groups at each end of the linker, such that one reactive group can be first reacted with the cytotoxic compound to provide a compound bearing the linker moiety and a second reactive group, which can then react with the antibody. Alternatively, one end of the bifunctional crosslinking agent can be first reacted with the antibody to provide an antibody bearing a linker moiety and a second reactive group, which can then react with the cytotoxic compound. The linking moiety may contain a chemical bond that allows for the release of the cytotoxic moiety at a particular site. Suitable chemical bonds are well known in the art and include disulfide bonds, thioether bonds, acid labile bonds, photolabile bonds, protease/peptidase labile bonds, and esterase labile bonds. See, for example, U.S. Pat. Nos. 5,208,020; 5,475,092; 6,441,163; 6,716,821; 6,913,748; 7,276,497; 7,276,499; 7,368,565; 7,388,026 and 7,414,073. In some embodiments, the bonds are disulfide bonds, thioether, and/or protease/peptidase labile bonds. Other linkers that can be used in the present invention include non-cleavable linkers, such as those described in detail in US 20050169933, charged linkers, or hydrophilic linkers, such as those described in US 2009/0274713, US 2010/0129314, and WO 2009/134976, each of which is expressly incorporated herein by reference.

In some embodiments, the linker is cleavable by a cleaving agent that is present in the intracellular environment (e.g., within a lysosome or endosome or caveolea). The linker can be, e.g., a peptide linker that is cleaved by an intracellular peptidase or protease enzyme, including, but not limited to, a lysosomal or endosomal protease. In some embodiments, the peptide linker comprises at least two, at least three, at least four, or at least five amino acids long. In certain embodiments, the peptide linker is selected from Gly-Gly-Gly, Ala-Val, Val-Ala, Val-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu, B-Ala-Leu-Ala-Leu, Gly-Phe-Leu-Gly, Val-Arg, Arg-Val, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, and Met-Ala. In some embodiments, the peptide linker is selected from Gly-Gly-Gly, Ala-Val, Ala-Ala, Ala-D-Ala, D-Ala-Ala, and D-Ala-D-Ala. Cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). One advantage of using intracellular proteolytic release of the cytotoxic drug agent is that the agent is typically attenuated when conjugated, and the serum stabilities of the conjugates are typically high.

In other embodiments, the cleavable linker is pH-sensitive, i.e., sensitive to hydrolysis at certain pH values. In some embodiments, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used (see, e.g., U.S. Pat. Nos. 5,122,368; 5,824,805; 5,622,929; Dubowchik and Walker, 1999, Pharm. Therapeutics 83:67-123; Neville et al, 1989, Biol. Chem. 264: 14653-14661). Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In other embodiments, the linker is cleavable under reducing conditions (e.g., a disulfide linker). Bifunctional crosslinking agents that enable the linkage of an antibody with cytotoxic compounds via disulfide bonds include, but are not limited to, N-succinimidyl-4-(4-nitropyridyl-2-dithio)butanoate, N-succinimidyl-3-(2-pyridyldithio)propionate (SPDP), N-succinimidyl-4-(2-pyridyldithio)pentanoate (SPP), N-succinimidyl-4-(2-pyridyldithio)butanoate (SPDB), N-succinimidyl-4-(2-pyridyldithio)-2-sulfo butanoate (sulfo-SPDB). Sulfo-SPDB is described, e.g., in U.S. Pat. No. 8,236,319, incorporated herein by reference. Alternatively, crosslinking agents that introduce thiol groups such as 2-iminothiolane, homocysteine thiolactone, or S-acetylsuccinic anhydride can be used. In other embodiments, the linker may contain a combination of one or more of the peptide, pH-sensitive, or disulfide linkers described previously.

“Heterobifunctional crosslinking agents” are bifunctional crosslinking agents having two different reactive groups. Heterobifunctional crosslinking agents containing both an amine-reactive N-hydroxysuccinimide group (NHS group) and a carbonyl-reactive hydrazine group can also be used to link cytotoxic compounds with an antibody. Examples of such commercially available heterobifunctional crosslinking agents include succinimidyl 6-hydrazinonicotinamide acetone hydrazone (SANH), succinimidyl 4-hydrazidoterephthalate hydrochloride (SHTH) and succinimidyl hydrazinium nicotinate hydrochloride (SHNH). Conjugates bearing an acid-labile linkage can also be prepared using a hydrazine-bearing benzodiazepine derivative of the present invention. Examples of bifunctional crosslinking agents that can be used include succinimidyl-p-formyl benzoate (SFB) and succinimidyl-p-formylphenoxyacetate (SFPA).

The present invention provides antibody-drug conjugates comprising one or more cytotoxic drug agents linked to a single antibody. The drug-to-antibody ratio (DAR) represents the number of cytotoxic drug agents linked per antibody molecule. In various embodiments, the DAR ranges from 1 to 15, 1 to 10, 1 to 9, 1 to 8, 1 to 7, 1 to 6, 1 to 5, 1 to 4, 1 to 3, or 1 to 2. In some embodiments, the DAR ranges from 2 to 10, 2 to 9, 2 to 8, 2 to 7, 2 to 6, 2 to 5, 2 to 4 or 2 to 3. In other embodiments, the DAR is about 2, about 2.5, about 3, about 4, about 5, or about 6. In some embodiments, the DAR ranges from about 2 to about 4. The DAR may be characterized by conventional means such as mass spectrometry, UV/Vis spectroscopy, ELISA assay, and/or HPLC.

The present invention includes the method of preparing antibody-drug conjugates. In some embodiments, the conjugates of the present invention are prepared by contacting the antibody with a cross-linking agent (linker) and a cytotoxic agent in a sequential manner, such that the antibody is covalently linked to a linker first, and then the pre-formed antibody-linker intermediate reacts with a cytotoxic agent. The antibody-linker intermediate may or may not be subjected to a purification step prior to contacting a cytotoxic agent. In some embodiments, the conjugates of the invention can be prepared by contacting the antibody with a cytotoxic agent-linker compound preformed by reacting the linker and the cytotoxic agent. The pre-formed linker-cytotoxic agent may or may not be subjected to a purification step prior to contacting the antibody. In other embodiments, the antibody contacts a linker and a cytotoxic agent in one reaction mixture, allowing simultaneous formation of the covalent bonds between the antibody and the linker, and between the linker and the cytotoxic agent. This method of preparing antibody-drug conjugates may include a reaction, wherein the antibody contacts a cytotoxic agent prior to the addition of a linker to the reaction mixture, and vice versa. In certain embodiments, the antibody-drug conjugate of the invention can be prepared by contacting the antibody with a cytotoxic agent having a built in linker such as, e.g., CDA-3.

The method of preparing antibody-drug conjugates includes buffer solutions having a pH of 3 to 9. In some embodiments, the buffer solution is at pH 4 to 9. In some embodiments, a pH of the buffer solution is between 7 and 9. In some embodiments, a pH of the buffer solution is between 8 and 9. In some embodiments, a pH of the buffer solution is 8.0. In other embodiments, a pH of the buffer solution is at 8.7.

The method of preparing antibody-drug conjugates includes buffer solutions with various ionic strengths. In some embodiments, the ionic strength of the buffer solution is between 10 mM and 300 mM. In some embodiments, the ionic strength of the buffer solution is between 15 mM and 200 mM. In some embodiments, the ionic strength of the buffer solution is between 60 mM and 150 mM. In some embodiments, the ionic strength of the buffer solution is 75 mM. In other embodiments, the ionic strength of the buffer solution is 130 mM.

In certain embodiments, the method of preparing antibody-drug conjugates includes buffer solutions with various concentrations. In some embodiments, the concentration of the buffer solution is between 10 mM and 300 mM. In some embodiments, the concentration of the buffer solution is between 15 mM and 200 mM. In some embodiments, the concentration of the buffer solution is between 60 mM and 150 mM. In some embodiments, the concentration of the buffer solution is 75 mM. In other embodiments, the concentration of the buffer solution is 130 mM.

The method of preparing antibody-drug conjugates utilizes any buffers known in the art, or any combination thereof. Examples of buffers are listed in the website for Sigma Aldrich at http://www.sigmaaldrich.com/life-science/core-bioreagents/biological-buffers/learning-center/buffer-reference-center.html. Examples of buffers also include, but not limited to, phosphate buffer, citrate buffer, succinate buffer, and acetate buffer. In some embodiments, the buffer solution is HEPES (4-(2-Hydroxyethyl)piperazine-1-ethanesulfonic acid). In other embodiments, the buffer solution is EPPS (4-(2-Hydroxyethyl)-1-piperazinepropanesulfonic acid).

The method of preparing antibody-drug conjugates includes organic solvents, for example, but not limited to, DMA (dimethylacetamide), and DMSO (dimethyl sulfoxide). In some embodiments, organic solvent is present in the conjugation reaction in the amount of 1 to 40% by volume of the total volume of the buffer solution and the organic solvent. In some embodiments, the organic solvent is DMA, and is present in the amount of 5-20%. In some embodiments, the organic solvent is DMA, and is present in the amount of 10%. In other embodiments, the organic solvent is DMA, and is present in the amount of 13.5%. In other embodiments, the organic solvent is DMA, and is present in the amount of 15%.

The method of preparing antibody-drug conjugates is carried out at a temperature between 2° C. and 37° C. In some embodiments, the temperature is between 10° C. and 30° C. In some embodiments, the temperature is between 15° C. and 25° C. In some embodiments, the temperature is 25° C. In other embodiments, the temperature is 22° C.

The method of preparing antibody-drug conjugates allows the conjugation reaction to proceed for 2 minutes to 2 days. In some embodiments, the reaction proceeds for 0.5 hour to 24 hours. In some embodiments, the reaction proceeds for 1 hour to 8 hours. In some embodiments, the reaction proceeds for 6 hours. In some embodiments, the reaction proceeds for 4 hours. In other embodiments, the reaction proceeds for 1 hour.

In some embodiments, the method of preparing antibody-drug conjugates of the invention further comprises the step of adding a quenching solution with high ionic strength after the formation of the conjugate. In one embodiment, the quenching solution comprises 750 mM EPPS and 150 mM of histidine hydrochloride. In another embodiment, the quenching solution comprises 750 mM EPPS. In some embodiments, the pH of the quenching solution is between 5 and 6. In some embodiments, the pH of the quenching solution is 5.5.

In some embodiments, the quenching solution comprises EPPS and histidine hydrochloride and subsequent to the addition of the quenching solution to the conjugation reaction mixture, the resulting mixture comprises 200 mM to 400 mM EPPS and 40-60 mM histidine hydrochloride. In one embodiment, the resulting mixture comprises 250 mM to 350 mM EPPS and 40-60 mM histidine hydrochloride. In another embodiment, the resulting mixture comprises 300 mM to 350 mM EPPS and 45 mM to 55 mM histidine hydrochloride.

The antibody-drug conjugates prepared according to the methods described above may be subjected to a purification step. The purification step involves any biochemical methods known in the art for purifying proteins, or any combination of methods thereof. These include, but not limited to, tangential flow filtration (TFF), affinity chromatography, ion exchange chromatography, any charge or isoelectric point-based chromatography, mixed mode chromatography, e.g., CHT (ceramic hydroxyapatite), hydrophobic interaction chromatography, size exclusion chromatography, dialysis, filtration, selective precipitation, or any combination thereof.

### Pharmaceutical Compositions

In another aspect, the invention features compositions, e.g., pharmaceutically acceptable compositions, which include an antibody-drug conjugate of the invention, as described herein, formulated together with a pharmaceutically acceptable carrier.

As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion). The pharmaceutical composition can include one or more additional excipients, e.g., salts, buffers, tonicity modifiers, lyoprotectants, nonionic detergents, surfactants, and preservatives. In some embodiments, the formulation buffer comprises a range of 5 mM to 300 mM of pharmaceutically acceptable buffer including, but not limited to, histidine, succinate, tris, or acetate at a range of pH 2.5 to 9.0. In other embodiments, the formulation buffer comprises excipients such as L-Proline, L-Arginine, cyclodextrins, e.g., gamma cyclodextrin, e.g., Captisol® and the likes thereof, polyethylene glycol, sucrose, trehalose, sodium bisulfite, or any other excipients that are known in the art to stabilize proteins or immunoconjugates, and minimize the formation of high molecular weight species or de-conjugation of drugs from the ADC, either during production or upon storage.

The compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Some typical compositions are in the form of injectable or infusible solutions, intended for parenteral administration (e.g., intravenous, subcutaneous, intraperitoneal, intramuscular). In some embodiments, the antibody is administered by intravenous infusion or injection. In other embodiments, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

In some embodiments, the pharmaceutical composition is sterile and stable under the conditions of manufacture and storage. The composition can be formulated as a solution, microemulsion, dispersion, liposome, microsphere, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by sterilization, e.g., by filtration. Generally, dispersions are prepared by incorporating the active compound into a sterile vehicle that contains a basic dispersion medium and the required other ingredients from those enumerated above. In the case of sterile powders for the preparation of sterile injectable solutions, the provided methods of preparation are vacuum drying and freeze-drying that yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibody-drug conjugates of the invention can be administered by a variety of methods known in the art, although for many therapeutic applications, the route/mode of administration is intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, antibody-drug conjugates described herein may be orally administered, for example, with an inert diluent or an assimilable edible carrier. The compound (and other ingredients if desired) may also be enclosed in a hard or soft shell gelatin capsule, compressed into tablets, buccal tablets, troches, capsules, elixirs, suspensions, syrups, wafers, and the like. To administer an antibody or an antibody fragment of the invention by other than parenteral administration, it may be necessary to coat the compound with, or co-administer the compound with, a material to prevent its inactivation.

Therapeutic compositions can be administered with medical devices known in the art. For example, pharmaceutical preparations can be disposed within a device, e.g., an air- or liquid-tight container, which contains one or more dosages. Examples of delivery devices include, without limitation, vials, cannulas, needles, drip bags, and lines. The invention also provides methods of placing an antibody-drug conjugate of the invention into such a device.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. The term “dosage unit form,” as used herein, refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an antibody or an antigen binding fragment of the invention is 20 μg-20 mg/kg, or 30 μg-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective” amount of an antibody-drug conjugate of the invention. A “therapeutically effective” amount refers to an amount effective, at dosages and for periods of time necessary, to achieve the desired therapeutic result. A therapeutically effective amount of an antibody-drug conjugate of the invention may vary according to factors such as the disease state, age, sex, and weight of the individual, and the ability of the antibody or antibody portion to elicit a desired response in the individual. A therapeutically effective amount is also one in which any toxic or detrimental effects of the antibody-drug conjugate is outweighed by the therapeutically beneficial effects. A “therapeutically effective dosage” preferably inhibits a measurable parameter (e.g., tumor growth rate) in treated subjects by at least about 20%, at least about 40%, at least about 60%, and in some embodiments at least about 80%, relative to untreated subjects. The ability of a compound to inhibit a measurable parameter, e.g., cancer, can be evaluated, e.g., in an animal model system predictive of efficacy in human tumors. Alternatively, this property of a composition can be evaluated in vitro assays such as, e.g., those described in Example 7.

Also within the scope of the invention are kits comprising an antibody-drug conjugate as described herein. The kit can include one or more other elements including: instructions for use; other reagents, e.g., a label, an additional therapeutic agent; devices or other materials for preparing the antibody-drug conjugate of the invention for administration; pharmaceutically acceptable carriers; and devices or other materials for administration to a subject. Instructions for use can include guidance for therapeutic application including suggested dosages and/or modes of administration, e.g., in a patient with a cancer (e.g., a cancer of gastrointestinal origin, such as, for example, colon cancer, stomach cancer, esophageal cancer).

The kit can further contain at least one additional reagent, such as an additional therapeutic agent, and/or one or more additional antibody-drug conjugates of the invention, formulated as appropriate, in one or more separate pharmaceutical preparations.

### Therapeutic Uses

As used herein, “treatment” or “treating” refers to an amelioration of a cancer or tumor, or at least one discernible symptom thereof. In certain embodiments, “treatment” or “treating” refers to an amelioration of at least one measurable physical parameter, not necessarily discernible by the patient. In yet another embodiment, “treatment” or “treating” refers to inhibiting the progression of a cancer, either physically, e.g., stabilization of a discernible symptom, physiologically, e.g., stabilization of a physical parameter, or both. “Treatment” or “treat” as used herein refers to the administration of an antibody-drug conjugate of the invention to a subject, e.g., a patient, or administration, e.g., by application, to an isolated tissue or cell from a subject which is returned to the subject. The antibody-drug conjugate can be administered alone or in combination with an additional therapeutic agent. The treatment can be to cure, heal, alleviate, relieve, alter, remedy, ameliorate, palliate, improve or affect the disorder, the symptoms of the disorder or the predisposition toward the disorder, e.g., a cancer. While not wishing to be bound by theory, treating is believed to cause the inhibition, ablation, or killing of a cell in vitro or in vivo, or otherwise reducing capacity of a cell, e.g., an aberrant cell, to mediate a disorder, e.g., a disorder as described herein (e.g., a cancer).

As used herein, the term “subject” is intended to include mammals, primates, humans and non-human animals. For example, a subject can be a patient (e.g., a human patient or a veterinary patient), having a cancer, e.g., of gastrointestinal origin (e.g., colon cancer), a patient having a symptom of a cancer, e.g., of gastrointestinal origin (e.g., colon cancer), in which at least some of the cells express GCC, or a patient having a predisposition toward a cancer, e.g., of gastrointestinal origin (e.g., colon cancer), in which at least some of the cells express GCC. The term “non-human animals” of the invention includes all non-human vertebrates, e.g., non-human mammals and non-mammals, such as non-human primates, sheep, dog, cow, chickens, amphibians, reptiles, etc., unless otherwise noted. In an embodiment, a subject excludes one or more or all of a mouse, rat, rabbit or goat.

As used herein, an amount of an antibody-drug conjugate “effective” or “sufficient” to treat a disorder, or a “therapeutically effective amount” or “therapeutically sufficient amount” refers to an amount of the antibody-drug conjugate which is effective, upon single or multiple dose administrations to a subject suffering from a disorder described herein, in treating a cell, e.g., cancer cell (e.g., a GCC-expressing tumor cell), in reducing tumor size or inhibiting the growth of a tumor or cancer in a subject, in prolonging a subject's survival, or in alleviating, relieving or improving one or more of a subject's symptoms beyond that expected in the absence of such treatment. As used herein, “inhibiting the growth” of the tumor or cancer refers to slowing, interrupting, arresting or stopping its growth and/or metastases and does not necessarily indicate a total elimination of the tumor growth.

In one aspect, the invention features a method of killing, inhibiting or modulating the growth of, or interfering with the metabolism of, a GCC-expressing cell by administering an antibody-drug conjugate of the invention. In one embodiment, the invention provides a method of inhibiting GCC-mediated cell signaling or a method of killing a cell. The method may be used with any cell or tissue which expresses GCC, such as a cancerous cell or a metastatic lesion. Non-limiting examples of GCC-expressing cancers include colon cancer, stomach cancer, esophageal cancer, pancreatic cancer, bladder cancer, cervical cancer, head and neck cancer, liver cancer, lung cancer and rectum cancer. Non-limiting examples of GCC-expressing cells include T84 human colonic adenocarcinoma cells, fresh or frozen colonic tumor cells, and cells comprising a recombinant nucleic acid encoding GCC or a portion thereof.

Methods of the invention include the steps of contacting the cell with an antibody-drug conjugate of the invention, as described herein, in an effective amount, i.e., amount sufficient to kill the cell. The method can be used on cells in culture, e.g. in vitro, in vivo, ex vivo, or in situ. For example, cells that express GCC (e.g., cells collected by biopsy of a tumor or metastatic lesion; cells from an established cancer cell line; or recombinant cells), can be cultured in vitro in culture medium and the contacting step can be effected by adding the antibody-drug conjugate of the invention to the culture medium. The method will result in killing of cells expressing GCC, including in particular tumor cells expressing GCC (e.g., colonic tumor cells).

The antibody portion of the antibody-drug conjugates of the invention bind to the extracellular domain of GCC or portions thereof in cells expressing the antigen. As a result, when practicing the methods of the present invention to kill, suppress, or detect cancerous cells, the antibody portion of the antibody-drug conjugate binds to all such cells, not only to cells which are fixed or cells whose intracellular antigenic domains are otherwise exposed to the extracellular environment. Consequently, binding is concentrated in areas where there are cells expressing GCC, irrespective of whether these cells are fixed or unfixed, viable, or necrotic.

The method also can be performed on cells present in a subject, as part of an in vivo protocol. In one embodiment, the subject is a human subject. Alternatively, the subject can be a mammal expressing a GCC antigen with which an antibody-drug conjugate of the invention cross-reacts. An antibody-drug conjugate of the invention also can be administered to a non-human mammal expressing the GCC-like antigen with which the antibody cross-reacts (e.g., a primate, pig or mouse) for veterinary purposes or as an animal model of human disease. Animal models may be useful for evaluating the therapeutic efficacy of antibodies of the invention (e.g., testing of dosages and time courses of administration). For in vivo embodiments, the contacting step is effected in a subject and includes administering an antibody-drug conjugate of the invention to the subject under conditions effective to permit both binding of the antibody molecule to the extracellular domain of GCC expressed on the cell, and the treating of the cell.

In one embodiment, the invention provides a method of treating cancer by administering an antibody-drug conjugate of the invention to a patient in need of such treatment. The method can be used for the treatment of any cancerous disorder which includes at least some cells that express the GCC antigen. As used herein, the term “cancer” is meant to include all types of cancerous growths or oncogenic processes, metastatic tissues or malignantly transformed cells, tissues, or organs, irrespective of histopathologic type or stage of invasiveness. The terms “cancer” and “tumor” may be used interchangeably (e.g., when used in the context of treatment methods, “treatment of a cancer” and “treatment of a tumor” have the same meaning).

In some embodiments, the treatment is sufficient to reduce or inhibit the growth of the subject's tumor, reduce the number or size of metastatic lesions, reduce tumor load, reduce primary tumor load, reduce invasiveness, prolong survival time, and/or maintain or improve the quality of life.

Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting the colon, bladder, cervix, esophagus, head and neck, liver, lung, rectum, stomach and pancreas. Carcinomas include, for example, bladder urothelial carcinoma, cervical squamous cell carcinoma, esophageal carcinoma, head and neck squamous cell carcinoma, liver hepatocellular carcinoma and lung cell carcinoma. Adenocarcinomas include, for example, malignancies such as non-small cell carcinoma of the lung, endocervical adenocarcinoma, colon adenocarcinoma, pancreatic adenocarcinoma, rectum adenocarcinoma and gastric adenocarcinoma. Metastatic lesions of the aforementioned cancers can also be treated or prevented using the methods and compositions of the invention. In some embodiments, the cancer to be treated is a cancer of the gastrointestinal system (e.g., colorectal cancer, colon cancer, rectal cancer, esophageal cancer, gastroesophageal cancer or stomach cancer). In some embodiments, the cancer to be treated is pancreatic cancer.

In one embodiment, the cancer is a colorectal cancer, e.g., colorectal adenocarcinoma, colorectal leiomyosarcoma, colorectal lymphoma, colorectal melanoma, or a colorectal neuroendocrine tumor. In a particular embodiment, the cancer is metastatic colon cancer. In another embodiment, the cancer is a stomach cancer (e.g., gastric adenocarcinoma, lymphoma, or sarcoma), or metastasis thereof. In another embodiment, the cancer is an esophageal cancer (e.g., a squamous cell carcinoma or adenocarcinoma of the esophagus).

The method can be useful in treating a relevant disorder at any stage or subclassification. For example, method can be used to treat early or late stage colon cancer, or colon cancer of any of stages 0, I, IIA, IIB, IIIA, IIIB, IIIC, and IV.

In some embodiments, an antibody-drug conjugate of the invention is administered in treatment cycles. A “treatment cycle” consists of a treatment period, during which the antibody-drug conjugate of the invention is administered as described above, followed by a rest period, during which no antibody-drug conjugate of the invention is administered. The treatment cycle can be repeated as necessary to achieve the desired effect.

The antibody-drug conjugates described herein may be used in combination with other therapies. For example, the combination therapy can include a composition of the present invention co-formulated with, and/or co-administered with, one or more additional therapeutic agents, e.g., one or more anti-cancer agents, e.g., additional cytotoxic or cytostatic agents, hormone treatment, vaccines, and/or other immunotherapies. In other embodiments, the antibody-drug conjugates of the invention are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concurrent” delivery. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the antibody-drug conjugate of the invention is used in combination with a chemotherapeutic agent. Non-limiting examples of DNA damaging chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitabine, gemcitabine, fludarabine, cytarabine, mercaptopurine, thioguanine, pentostatin, and hydroxyurea).

Combination therapies may include chemotherapeutic agents that disrupt cell replication, such as: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib, ixazomib, carfilzomib); NF-κB inhibitors, including inhibitors of IκB kinase; antibodies which bind to proteins overexpressed in cancers and thereby downregulate cell replication (e.g., trastuzumab, rituximab, cetuximab, and bevacizumab); and other inhibitors of proteins or enzymes known to be upregulated, over-expressed or activated in cancers, the inhibition of which downregulates cell replication.

The selection of therapeutic agent(s) or treatment modality to be combined with an antibody-drug conjugate of the invention will depend on the disorder to be treated. The additional agent(s) or treatment modality may include, for example, standard approved therapies for the indication being treated. For example, when the antibody-drug conjugate of the invention is used to treat colon cancer, it may be used in combination with, e.g., surgery; radiation therapy; 5-fluorouracil (5-FU), capecitibine, leucovorin, irinotecan, oxaliplatin, bevacizumab, cetuximab, panitumum, or combinations thereof (e.g., oxaliplatin/capecitibine (XELOX), 5-fluorouracil/leucovorinl-oxaliplatin (FOLFOX), 5-fluorouracil/leucovorin/irinotecan (FOLFIRI), FOLFOX plus bevacizumab, or FOLFIRI plus bevacizumab).

In another aspect, the invention features the use of an antibody-drug conjugate of the invention in the manufacture of a medicament. In an embodiment, the medicament is for treating cancer, e.g., a gastrointestinal cancer, e.g., colorectal cancer, esophageal cancer, or stomach cancer. In some embodiments, the cancer is pancreatic cancer. In one embodiment, the medicament is used to treat a colorectal cancer, e.g., colorectal adenocarcinoma, colorectal leiomyosarcoma, colorectal lymphoma, colorectal melanoma, or a colorectal neuroendocrine tumor. In a particular embodiment, the medicament is used to treat metastatic colon cancer. In another embodiment, the medicament is used to treat stomach cancer (e.g., gastric adenocarcinoma, lymphoma, or sarcoma), or metastasis thereof. In another embodiment, the medicament is used to treat an esophageal cancer (e.g., a squamous cell carcinoma or adenocarcinoma of the esophagus).

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The Examples do not in any way limit the disclosure.

### Example 1: Generating an Antibody Production Cell Line

To generate a stable Chinese hamster ovary (CHO) cell line clone expressing 5F9 with a productivity of >600 mg/L, expression vectors for 5F9 were generated by subcloning light chain variable region (SEQ ID NO:8) and heavy chain variable region (SEQ ID NO:7) into the pLKTOK58 expression vector, containing WT human IgG1 Fc and the neomycin resistance gene. Expression of the 5F9 variable region-IgG1 fusion product is under control of the EF-1α promoter.

### Cloning and Sequencing of the Anti-GCC Human Monoclonal Antibody 5F9 Variable Regions

Total RNA was isolated (Qiagen's RNeasy kit) from human hybridoma 46.5F9 subclone 8.2. This hybridoma carries the “standard” published Kappa constant region of the light chain (GenBank Accession Nos. AW383625 or BM918539) and the “standard” published IgG2 constant region of the heavy chain (GenBank Accession Nos. BX640623 or AJ294731). 5′ race-ready, poly-G tailed cDNA was synthesized by traditional methods (Nature Methods, 2:629-630 (2005)). The light chain variable region was PCR amplified from cDNA by 5′ race using a poly-C anchor oligo in combination with a reverse primer specific for the Kappa constant region. The heavy chain variable region was amplified with a reverse primer specific for the IgG2 constant region in multiple combinations with forward primers specific to the known heavy chain leader sequences. PCR products were TOPO® cloned (Invitrogen™, Life Technologies, Inc.) and sequenced with M13F and M13R primers.

### Construction of Mammalian Expression Vectors Carrying Anti-GCC Human Monoclonal Antibody 5F9

Mammalian expression vectors carrying the 5F9 light and heavy variable regions were constructed to generate production CHO cell lines. For the native construct, the variable regions of the 5F9 light and heavy chains were sub-cloned into pLKTOK58D (US Patent Application No. 20040033561). This vector carries two mammalian selection markers, including neomycin resistance and DHFR/methotrexate (for amplification). The vector allows co-expression of both light and heavy chains from tandem EF-1α promoters, each located upstream of the vector's leader-Kappa constant and leader-IgG1 (wild type Fc) constant regions. For sub-cloning, the variable regions of the light and heavy chains were PCR amplified from sequence-confirmed TOPO clones with gene-specific primers containing unique restriction sites for directional cloning into the junctions of the respective leader-Kappa and leader-IgG1 regions of the vector. The sequences of the primers are as follows (5F9 variable region-specific sequences are in bold font):

### Native 5F9 Light Chain Leader-Variable Primers:

Forward NotI
(SEQ ID NO: 13)
5′
ataagaatGCGGCCGCCTCACCATGGGATGGAGCTGTATCATCCTCTTCT
TGGTAGCAAC
AGCTACAGGTGTCCACTCCGAAATAGTGATGACGCAGTCTCCAGCCACCC
TG-3′
Reverse BsiWI
(SEQ ID NO: 14)
5′-GCCACCGTACGTTTGATTTCCACGTTGGTCCCTTGGCCGAACGTC-
3′

### Native 5F9 Heavy Chain Leader-Variable Primers:

Forward EcoRI
(SEQ ID NO: 15)
5′-
ccgGAATTCCTCACCATGGGATGGAGCTGTATCATCCTCTTCTTGGTAGC
AACAGCT ACAGGTGTCCACTCCCAGGTGCAGCTACAGCAGTGGGGCGCA
GGAC-3′
Reverse BlpI
(SEQ ID NO: 16)
5′-GGAGGCTGAGCTGACGGTGACCAGGGTTCCCTGGCCCCAGTGGTC-
3′

Clones were confirmed by double stranded DNA sequencing of both the light and heavy chains.

Two transfection methods were used to introduce the constructs into CHO cells: the traditional MPI process and the Crucell process. CHO cell transfections were initiated with the native 5F9 construct using the traditional MPI process. Linearized and nonlinearized DNAs were used, with either electroporation or Lipopfectamine 2000 CD transfection. Approximately 30 stable pools were generated through selection in G418, non-nucleoside medium, and 5 nM methotrexate. Based on FMAT analysis of antibody production levels, three stable pools were chosen for cloning. The pool with the highest production secreted antibody at 12.2 μg/mL. These three pools were frozen down.

Crucell STAR elements can be evaluated to make 5F9 expression vectors containing a STAR element.

The heavy and light chain nucleic acid sequences for 5F9 listed below were inserted into pTOK58D vector.

5F9 Heavy Chain Nucleic Acid Sequence in pTOK58D Vector:

(SEQ ID NO: 17)
atgggatggagctgtatcatcctcttcttggtagcaacagctacaggtgt
ccactcccaggtgcagctacagcagtggggcgcaggactgttgaagcctt
cggagaccctgtccctcacctgcgctgtctttggtgggtctttcagtggt
tactactggagctggatccgccagcccccagggaaggggctggagtggat
tggggaaatcaatcatcgtggaaacaccaacgacaacccgtccctcaaga
gtcgagtcaccatatcagtagacacgtccaagaaccagttcgccctgaag
ctgagttctgtgaccgccgcggacacggctgtttattactgtgcgagaga
acgtggatacacctatggtaactttgaccactggggccagggaaccctgg
tcaccgtcagctcagcctccaccaagggcccatcggtcttccccctggca
ccctcctccaagagcacctctgggggcacagcggccctgggctgcctggt
caaggactacttccccgaaccggtgacggtgtcgtggaactcaggcgccc
tgaccagcggcgtgcacaccttcccggctgtcctacagtcctcaggactc
tactccctcagcagcgtggtgaccgtgccctccagcagcttgggcaccca
gacctacatctgcaacgtgaatcacaagcccagcaacaccaaggtggaca
agaaagttgagcccaaatcttgtgacaaaactcacacatgcccaccgtgc
ccagcacctgaactcctggggggaccgtcagtcttcctcttccccccaaa
acccaaggacaccctcatgatctcccggacccctgaggtcacatgcgtgg
tggtggacgtgagccacgaagaccctgaggtcaagttcaactggtacgtg
gacggcgtggaggtgcataatgccaagacaaagccgcgggaggagcagta
caacagcacgtaccgtgtggtcagcgtcctcaccgtcctgcaccaggact
ggctgaatggcaaggagtacaagtgcaaggtctccaacaaagccctccca
gcccccatcgagaaaaccatctccaaagccaaagggcagccccgagaacc
acaggtgtacaccctgcccccatcccgggatgagctgaccaagaaccagg
tcagcctgacctgcctggtcaaaggcttctatcccagcgacatcgccgtg
gagtgggagagcaatgggcagccggagaacaactacaagaccacgcctcc
cgtgctggactccgacggctccttcttcctctacagcaagctcaccgtgg
acaagagcaggtggcagcaggggaacgtcttctcatgctccgtgatgcat
gaggctctgcacaaccactacacgcagaagagcctctccctgtctccggg
taaataa

5F9 Light Chain Nucleic Acid Sequence in pTOK58D Vector:

(SEQ ID NO: 18)
atgggatggagctgtatcatcctcttcttggtagcaacagctacaggtgt
ccactccgaaatagtgatgacgcagtctccagccaccctgtctgtctcca
ggggaaagagccaccctctcctgcagggccagtcagagtgttagcagaaa
cttagcctggtatcagcagaaacctggccaggctcccaggctcctcatct
atggtgcatccaccagggccactggaatcccagccaggttcagtggcagt
gggtctgggacagagttcactctcaccatcggcagcctgcagtctgaaga
ttttgcagtttattactgtcagcagtataaaacctggcctcggacgttcg
gccaagggaccaacgtggaaatcaaacgtacggtggctgcaccatctgtc
ttcatcttcccgccatctgatgagcagttgaaatctggaactgcctctgt
tgtgtgcctgctgaataacttctatcccagagaggccaaagtacagtgga
aggtggataacgccctccaatcgggtaactcccaggagagtgtcacagag
caggacagcaaggacagcacctacagcctcagcagcaccctgaccctgag
caaagcagactacgagaaacacaaagtctacgcctgcgaagtcacccatc
agggcctgagctcgcccgtcacaaagagcttcaacaggggagagtgttag

### Example 2: Preparation of Cytotoxic Drug Agent CDA-1

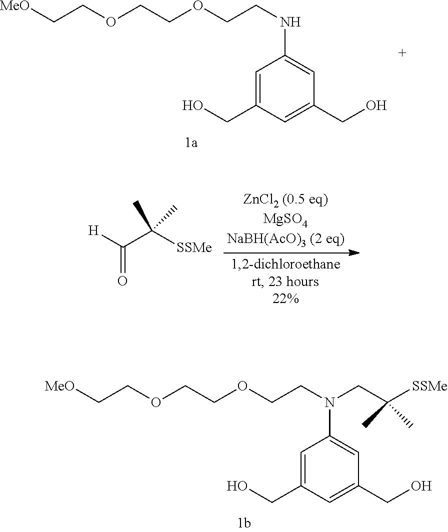

Sodium triacetoxyborohydride (1.1 g, 5.18 mmol) and zinc chloride powder (353 mg, 2.59 mmol) were added to a stirred solution of aniline 1a (1.55 g, 5.18 mmol) and 2-(methyldithio)-isobutyraldehyde (0.7 mL, 5.18 mmol) in anhydrous 1,2-dichloromethane (20 mL), followed by the addition of anhydrous magnesium sulfate (800 mg). The mixture was stirred at room temperature for 6 hours, and then a second portion of 2-(methyldithio)-isobutyraldehyde (0.7 mL, 5.18 mmol) and sodium triacetoxyborohydride (1.1 g, 5.18 mmol) was added. Stirring was continued at room temperature overnight. The reaction mixture was filtered through celite and washed with dichloromethane. The filtrate was concentrated and the remainder was purified by silica gel chromatography (Combiflash, 40 g column, dichloromethane/MeOH) to give compound 1b (487 mg y=22%) as colorless oil. Unreacted starting material aniline 1a (1.02 g) was also recovered in 65% yield. ¹H NMR (400 Hz, CDCl₃): δ6.76 (s, 2H), 6.63 (s, 1H), 4.55 (s, 4H), 3.65-3.51 (m, 14H), 3.35 (s, 3H), 2.44 (s, 3H), 1.33 (s, 6H); ¹³C NMR (400 Hz, CDCl₃): δ149.0, 142.35, 114.0, 111.1, 71.98, 70.7, 70.6, 70.5, 67.6, 65.5, 59.75, 59.1, 53.9, 51.9, 26.6, 25.7, 20.75; MS (m/z) found 456.2 (M+Na)⁺.

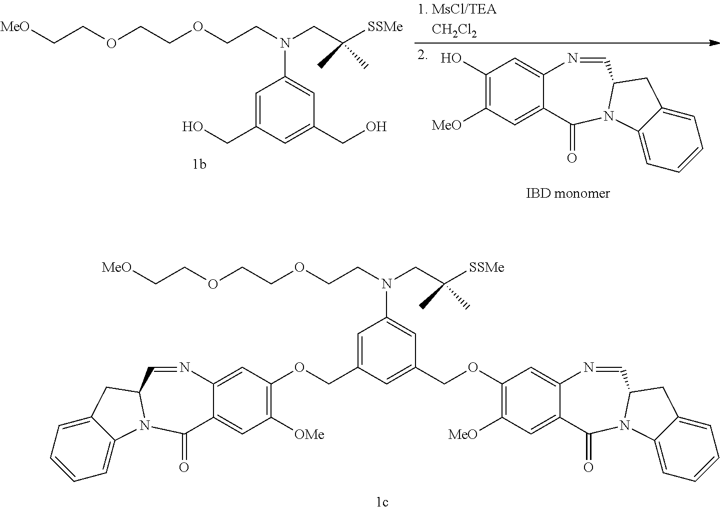

Trimethylamine (234 μL, 1.68 mmol) was added a stirred solution of compound 1b (243 mg, 0.56 mmol) in anhydrous dichloromethane (3.5 mL). The mixture was cooled to −10° C. and methanesulfonyl chloride (113 μL, 1.46 mmol) was added slowly over 15 min via syringe. The solution continued to be stirred for 60 min at −10 to −7° C. and quenched by addition of ice/water. It was then diluted with ethyl acetate and washed with cold water. The organic layer was dried over anhydrous sodium sulfate, filtered, concentrated, and high vacuumed to give the mesylates as a light yellowish oil (340 mg). The mesylates were transferred into a 10 mL round-bottomed flask with ethyl acetate/dichloromethane, concentrated, and high vacuumed. IBD monomer (412 mg, 1.4 mmol) was added followed by the addition of anhydrous dimethylformamide (3 mL) and anhydrous potassium carbonate (232 mg, 1.68 mmol). The obtained yellowish mixture was stirred at room temperature overnight, then diluted with dichloromethane and washed with brine. The organic layer was dried over anhydrous sodium sulfate, filtered, and concentrated. The residue was dissolved in dichloromethane, loaded on silica gel column, and eluted with dichloromethane/methanol (15:1 then 10:1). The fractions that contained compound 1c were combined and concentrated to give 705 mg of crude product which was further purified by preparative reverse phase HPLC (C-18 column, eluted with acetonitrile/water) to give compound 1c as a yellowish fluffy solid (181 mg, y=33%). 1H NMR (400 Hz, CDCl₃): δ8.28 (d, J=8.0 Hz, 2H), 7.86 (d, J=3.6 Hz, 2H), 7.59 (s, 2H), 7.31-7.26 (m, 4H), 7.12 (t, J=7.6 Hz, 2H), 6.87-6.80 (m, 5H), 5.18 (dd, J₁=20.8 Hz, J₂=12.4 Hz, 4H), 4.50-4.47 (m, 2H), 3.99 (s, 6H), 3.75-3.48 (m, 18H), 3.37 (s, 3H), 2.44 (s, 3H), 1.32 (s, 6H); MS (m/z) found 1025.9 (M+H₂O+Na)⁺, 1043.9 (M+2H₂O+Na)⁺, 983.8 (M−H)⁻, 1055.8 (M+4 H₂O—H)⁻.

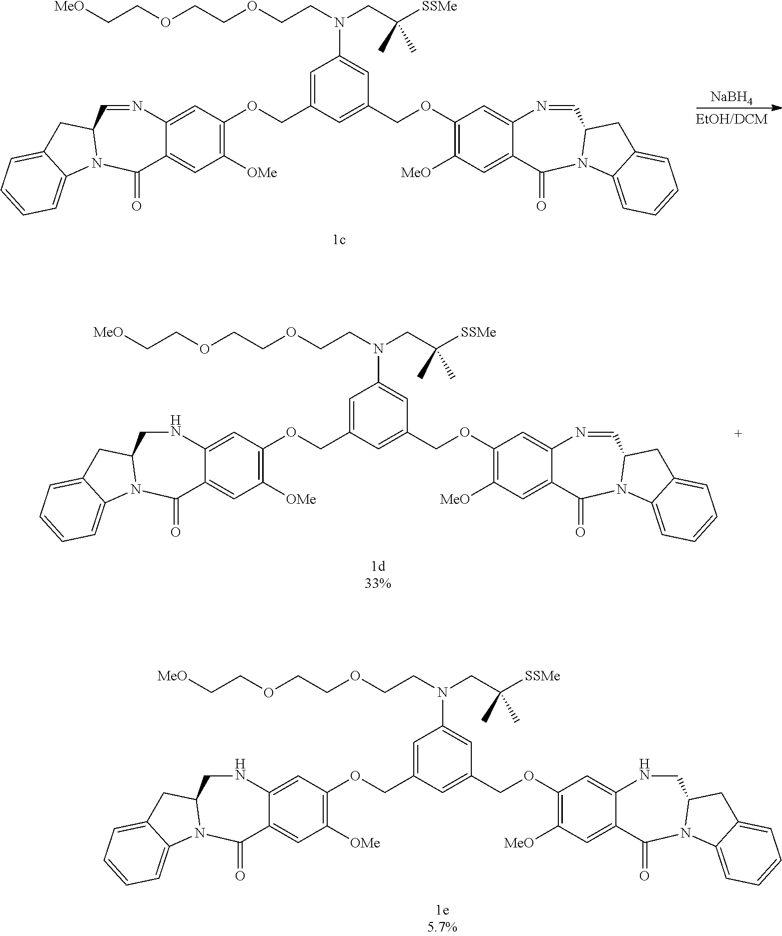

Sodium borohydride (0.9 mg, 0.023 mmol) was added to a stirred solution of compound 1c (112 mg, 0.114 mmol) in anhydrous dichloromethane (0.3 mL) and absolute ethanol (0.6 mL) at 0° C. The ice bath was removed after 5 min. The mixture was stirred at room temperature for 3 hours and cooled to 0° C. The mixture was quenched with saturated ammonium chloride, diluted with dichloromethane, and separated. The organic layer was washed with brine, dried over anhydrous sodium sulfate (Na₂S0₄), filtered through celite, and concentrated. The residue was purified by reverse phase HPLC (C-18 column, acetonitrile/water). The corresponding fractions were extracted with dichloromethane and concentrated to obtain the products 1d, 1e, and the unreacted starting material 1c. Compound 1d: 37.1 mg (y=33%), MS (m/z): found 1010.4 (M+Na)⁺, 1028.4 (M+H₂O+Na)⁺, 1040.3 (M+3H₂O—H)⁻; compound 1e: 6.4 mg (y=5.7%), MS (m/z): found 1012.4 (M+Na)⁺; compound 1c: 44.1 mg (y=39%).

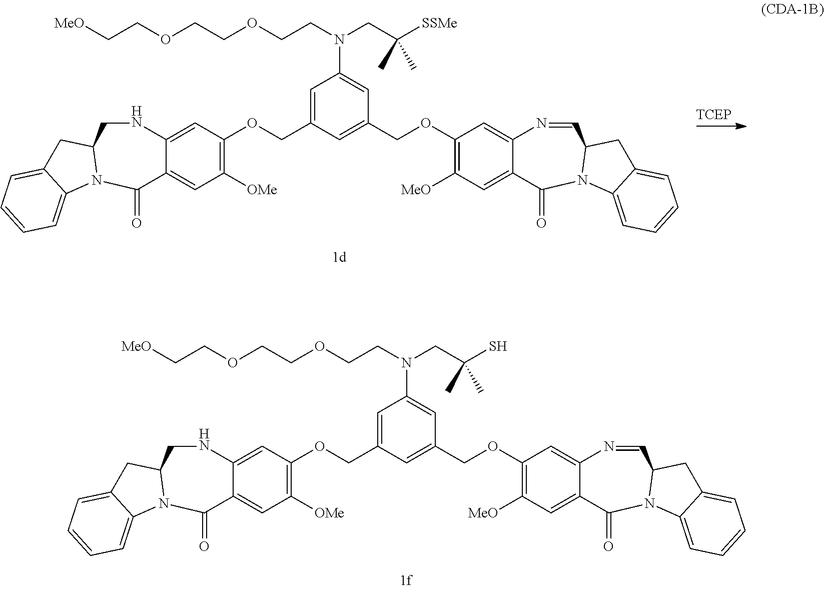

Freshly prepared TCEP solution (17 mg of TCEP HCl salt neutralized with saturated sodium bicarbonate to pH 6-6.5, then diluted with 0.5 mL of pH 6.5 phosphate buffer) was added to a stirred solution of compound 1d (23.6 mg, 0.024 mmol) in acetonitrile (3 mL) and methanol (3 mL) at room temperature. The mixture was stirred at room temperature for 3 hours, and then diluted with dichloromethane and deionized water and separated. The organic layer was washed with brine, dried over anhydrous sodium sulfate, and filtered. The filtrate was concentrated and high vacuumed to yield 22 mg of compound 1f (CDA-1B) as a light yellowish foam.

CDA-1A, the sulfonated form of CDA-1B can be prepared by treating CDA-1B with NaHSO₃. See exemplary reaction conditions for converting CDA-2B to CDA-2A in Example 3 below.

### Example 3: Preparation of Cytotoxic Drug Agent CDA-2

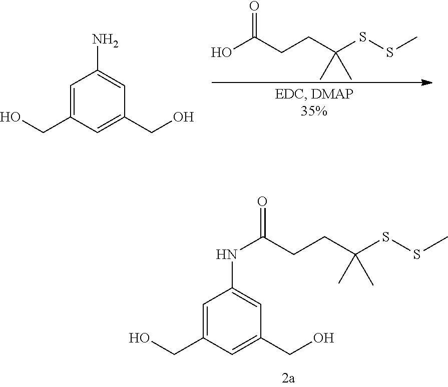 [SMILES: Nc1cc(CO)cc(CO)c1 ; CSSC(C)(C)CCC(=O)O ; CSSC(C)(C)CCC(=O)Nc1cc(CO)cc(CO)c1]

Compound (12S,12aS)-9-((3-(4-mercapto-4-methylpentanamido)-5-((((R)-8-methoxy-6-oxo-11,12,12a,13-tetrahydro-6H-benzo[5,6][1,4]diazepino[1,2-a]indol-9-yl)oxy)methyl)benzyl)oxy)-8-methoxy-6-oxo-11,12,12a,13-tetrahydro-6H-benzo[5,6][1,4]diazepino[1,2-a]indole-12-sulfonic acid (CDA-2A) was prepared as follows:

4-methyl-4-(methyldisulfanyl)pentanoic acid (1.281 g, 6.59 mmol), N-(3-dimethylaminopropyl)-N′-ethylcarbodiimide hydrochloride (2.53 g, 13.19 mmol), and 4-dimethylaminopyridine (0.081 g, 0.659 mmol) was added to a stirred solution of (5-amino-1,3-phenylene)dimethanol (1.01 g, 6.59 mmol) in anhydrous dimethylformamide (16.48 mL) and anhydrous tetrahydrofuran (16.48 ml). The resulting mixture was stirred for 18 hours at room temperature. The reaction was quenched with saturated ammonium chloride solution and extracted with ethyl acetate (3×50 mL). The organic extracts were washed with water and brine, then dried over anhydrous sodium sulfate. The solution was filtered and concentrated in vacuo and the resulting residue was purified by silica gel chromatography (ethyl acetate/hexanes) to obtain compound 2a as a white solid (0.70 g, 32% yield). 1H NMR (400 MHz, DMSO-d6: δ9.90 (s, 1H) 7.43 (s, 2H), 6.93 (s, 1H), 5.16 (t, 2H, J=5.7 Hz), 4.44 (d, 4H, J=5.7 Hz), 2.43 (s, 3H), 2.41-2.38 (m, 2H), 1.92-1.88 (m, 2H), 1.29 (s, 6H). MS (m/z): found 330.0 (M=1)¹.

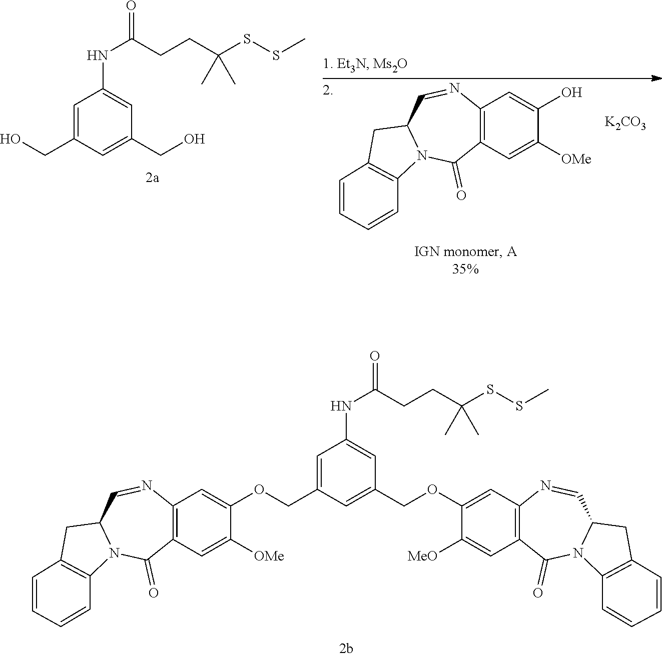

Trimethylamine (463 μl, 3.32 mmol) was added to a cooled (−10° C.) solution of compound 2a (219 mg, 0.665 mmol) in anhydrous dichloromethane (6.65 mL), followed by dropwise addition of methanesulfonic anhydride (298 mg, 1.662 mmol). The mixture stirred at −10° C. for 2 hours, then the mixture was quenched with ice water and extracted with cold ethyl acetate (2×30 mL). The organic extracts were sashed with ice water, dried with anhydrous sodium sulfate, filtered, and concentrated under reduced pressure to obtain the crude dimesylate.

The crude dimesylate (227 mg, 0.467 mmol) and IGN monomer A (303 mg, 1.028 mmol) were dissolved in anhydrous DMF (3.11 mL). Potassium carbonate (161 mg, 1.169 mmol) was added and the mixture stirred for 18 hours at room temperature. Deionized water was added and the resulting precipitate was filtered and rinsed with water. The solid was re-dissolved in dichloromethane and washed with water. The organic layer was dried with anhydrous magnesium sulfate, filtered, and concentrated. The crude residue was purified by silica gel chromatography (methanol/dichloro-methane) to give compound 2b (227 mg, 36% yield). MS (m/z): found 882.5 (M+1)⁺.

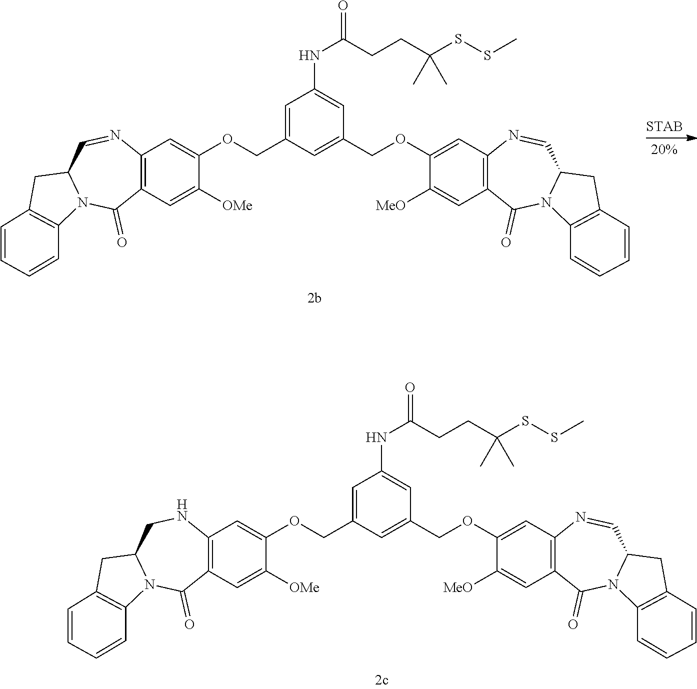 [SMILES: COc1cc2c(cc1OCc1cc(COc3cc4c(cc3OC)C(=O)N3c5ccccc5C[C@H]3C=N4)cc(NC(=O)CCC(C)(C)SSC)c1)N=C[C@@H]1Cc3ccccc3N1C2=O ; COc1cc2c(cc1OCc1cc(COc3cc4c(cc3OC)C(=O)N3c5ccccc5C[C@H]3CN4)cc(NC(=O)CCC(C)(C)SSC)c1)N=C[C@@H]1Cc3ccccc3N1C2=O]

Sodium triacetoxyborohydride (37.3 mg, 0.167 mmol) was added to a suspension of compound 2b (227 mg, 0.167 mmol) in anhydrous 1,2-dichloroethane (3.346 mL). The mixture was stirred at room temperature for 1 hour, after which it was quenched with saturated ammonium chloride solution. The mixture was extracted with dichloromethane and washed with brine. The organic layer was dried with anhydrous magnesium sulfate, filtered, and concentrated. The crude residue was purified by RP-HPLC (C-18, water/acetonitrile). Fractions containing desired product were extracted with dichloromethane, dried with anhydrous magnesium sulfate, filtered, and concentrated to give compound 2c (35 mg, 19% yield). MS (m/z), found 884.3 (M+1)⁺.

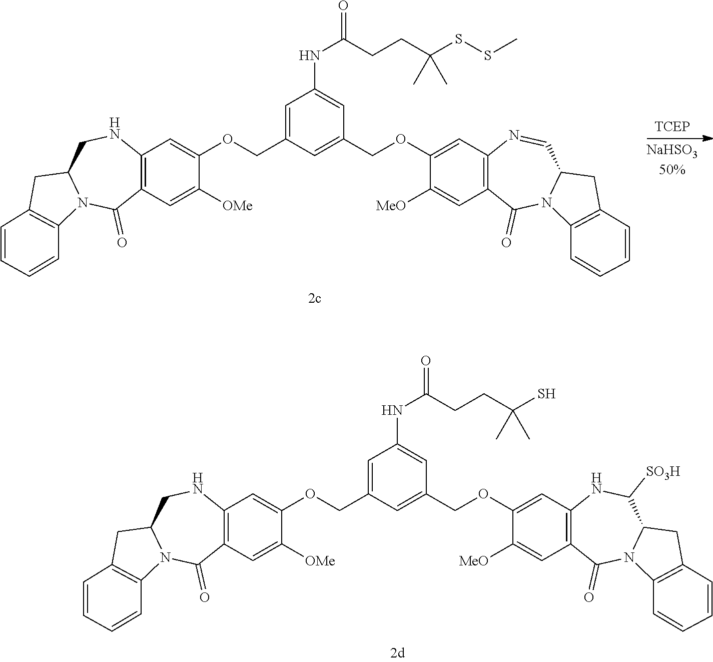

Tris(2-carboxyethyl)phosphine hydrochloride (17.51 mg, 0.060 mmol), neutralized with saturated sodium bicarbonate solution (0.2 mL) in sodium phosphate buffer (132 μL, 0.75 M, pH 6.5), was added to a solution of compound 2c (18 mg, 0.017 mmol) in acetonitrile (921 μL) and methanol (658 μL). The mixture was stirred at room temperature for 3.5 hours, then diluted with dichloromethane and deionized water. The organic layer was separated, washed with brine, dried with anhydrous sodium sulfate, filtered, and concentrated under reduced pressure to obtain the crude thiol (CDA-2B). MS (m/z), found 838.3 (M+1)⁺.

The crude thiol (CDA-2B) (15.5 mg, 0.018 mmol) was dissolved in 2-propanol (1.23 mL). Deionized water (617 μL) and sodium bisulfite (5.77 mg, 0.055 mmol) were then added, and the mixture was stirred for 5 hours at room temperature. The reaction was frozen in an acetone/dry ice bath, lyophilized, and purified by RP-HPLC (C-18, deionized water/acetonitrile). Fractions containing desired product were frozen and lyophilized to give compound (12S,12aS)-9-((3-(4-mercapto-4-methylpentanamido)-5-((((R)-8-methoxy-6-oxo-11,12,12a,13-tetrahydro-6H-benzo[5,6][1,4]diazepino[1,2-a]indol-9-yl)oxy)methyl)benzyl)oxy)-8-methoxy-6-oxo-11,12,12a,13-tetrahydro-6H-benzo[5,6][1,4]diazepino[1,2-a]indole-12-sulfonic acid (compound 2d or CDA-2A) (6.6 mg, 39% yield). MS (m/z), found 918.2 (M−1)⁻.

### Example 4: Preparation of Cytotoxic Drug Agent CDA-3

Synthesis of 2,5-dioxopyrrolidin-1-yl 6-(((S)-1-(((S)-1-((3-((((S)-8-methoxy-6-oxo-11,12,12a,13-tetrahydro-6H-benzo[5,6][1,4]diazepino[1,2-a]indol-9-yl)oxy)methyl)-5-((((R)-8-methoxy-6-oxo-12a,13-dihydro-6H-benzo[5,6][1,4]diazepino[1,2-a]indol-9-yl)oxy)methyl) phenyl)amino)-1-oxopropan-2-yl)amino)-1-oxopropan-2-yl)amino)-6-oxohexanoate (CDA-3B) was carried out as follows:

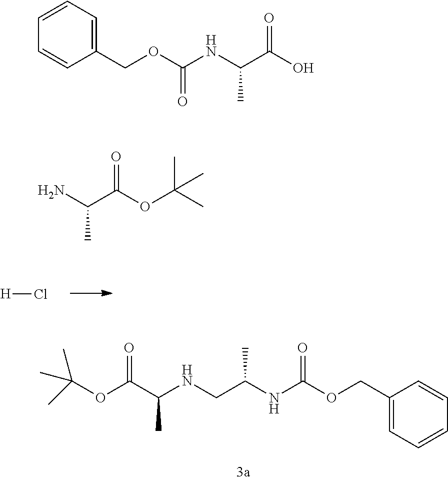

(S)-2-(((benzyloxy)carbonyl)amino)propanoic acid (5 g, 22.40 mmol) and (S)-tert-butyl 2-aminopropanoate hydrochloride (4.48 g, 24.64 mmol) were dissolved in anhydrous DMF (44.8 mL), and EDC.HCl (4.72 g, 24.64 mmol), HOBt (3.43 g, 22.40 mmol), and DIPEA (9.75 mL, 56.0 mmol) were added. The reaction was stirred under argon at room temperature overnight. The reaction mixture was diluted with dichloromethane and then washed with saturated ammonium chloride, saturated sodium bicarbonate, water, and brine. The organic layer was dried over sodium sulfate and concentrated. The crude oil was purified via silica gel chromatography (hexanes/ethyl acetate) to yield compound 3a (6.7 g, 85% yield). 1H NMR (400 MHz, CDCl₃): δ 7.38-7.31 (m, 5H), 6.53-6.42 (m, 1H), 5.42-5.33 (m, 1H), 5.14 (s, 2H), 4.48-4.41 (m, 1H), 4.32-4.20 (m, 1H), 1.49 (s, 9H), 1.42 (d, 3H, J=6.8 Hz), 1.38 (d, 3H, J=7.2 Hz).

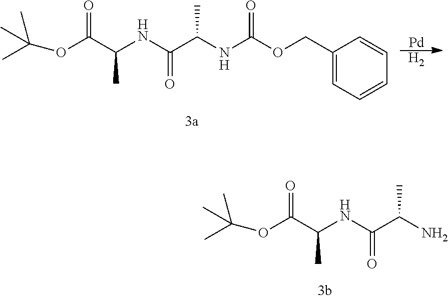

Compound 3a (6.7 g, 19.12 mmol) was dissolved in methanol (60.7 mL) and water (3.03 mL). The solution was purged with argon for 5 min. Palladium on carbon (wet, 10%) (1.017 g, 0.956 mmol) was added slowly. The reaction was stirred overnight under an atmosphere of hydrogen. The solution was filtered through celite, rinsed with methanol, and concentrated. It was then azeotroped with methanol and acetonitrile, and the resulting oil was placed directly on the high vacuum to give compound 3b (4.02 g, 97% yield). 1H NMR (400 MHz, CDCl₃): δ 7.78-7.63 (m, 1H), 4.49-4.42 (m, 1H), 3.55-3.50 (m, 1H), 1.73 (s, 2H), 1.48 (s, 9H), 1.39 (d, 3H, J=7.2 Hz), 1.36 (d, 3H, J=6.8 Hz).

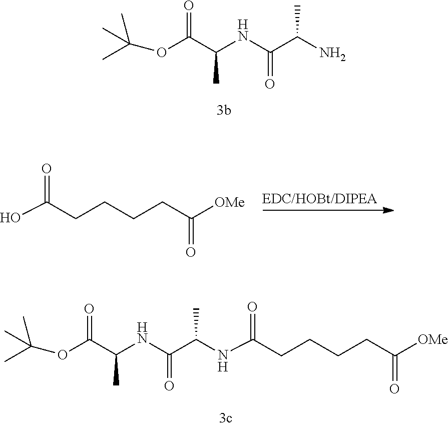 [SMILES: C[C@H](N)C(=O)N[C@@H](C)C(=O)OC(C)(C)C ; COC(=O)CCCCC(=O)O ; COC(=O)CCCCC(=O)N[C@@H](C)C(=O)N[C@@H](C)C(=O)OC(C)(C)C]

Compound 3b (4.02 g, 18.59 mmol) and mono methyladipate (3.03 mL, 20.45 mmol) were dissolved in anhydrous DMF (62.0 mL). EDC.HCl (3.92 g, 20.45 mmol), HOBt (2.85 g, 18.59 mmol) and DIPEA (6.49 mL, 37.2 mmol) were added. The mixture was stirred overnight at room temperature. The reaction was diluted with dichloromethane/methanol (150 mL, 5:1), and washed with saturated ammonium chloride, saturated sodium bicarbonate, and brine. It was dried over sodium sulfate, filtered, and stripped. The compound was azeotroped with acetonitrile (5×), then pumped on the high vacuum at 35° C. to give compound 3c (6.66 g, 100% yield). The crude material was taken onto next step without purification. 1H NMR (400 MHz, CDCl₃): δ 6.75 (d, 1H, J=6.8 Hz), 6.44 (d, 1H, J=6.8 Hz), 4.52-4.44 (m, 1H), 4.43-4.36 (m, 1H), 3.65 (s, 3H), 2.35-2.29 (m, 2H), 2.25-2.18 (m, 2H), 1.71-1.60 (m, 4H), 1.45 (s, 9H), 1.36 (t, 6H, J=6.0 Hz).

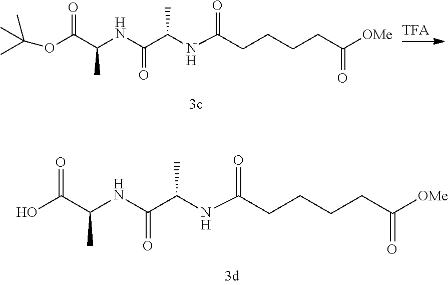 [SMILES: COC(=O)CCCCC(=O)N[C@@H](C)C(=O)N[C@@H](C)C(=O)OC(C)(C)C ; COC(=O)CCCCC(=O)N[C@@H](C)C(=O)N[C@@H](C)C(=O)O]

Compound 3c (5.91 g, 16.5 mmol) was stirred in TFA (28.6 mL, 372 mmol) and deionized water (1.5 mL) at room temperature for 3 hours. The reaction mixture was concentrated with acetonitrile and placed on high vacuum to give crude compound 3d as a sticky solid (5.88 g, 100% yield). 1H NMR (400 MHz, CDCl₃): δ 7.21 (d, 1H, J=6.8 Hz), 6.81 (d, 1H, J=7.6 Hz), 4.69-4.60 (m, 1H), 4.59-4.51 (m, 1H), 3.69 (s, 3H), 2.40-2.33 (m, 2H), 2.31-2.24 (m, 2H), 1.72-1.63 (m, 4H), 1.51-1.45 (m, 3H), 1.42-1.37 (m, 3H).

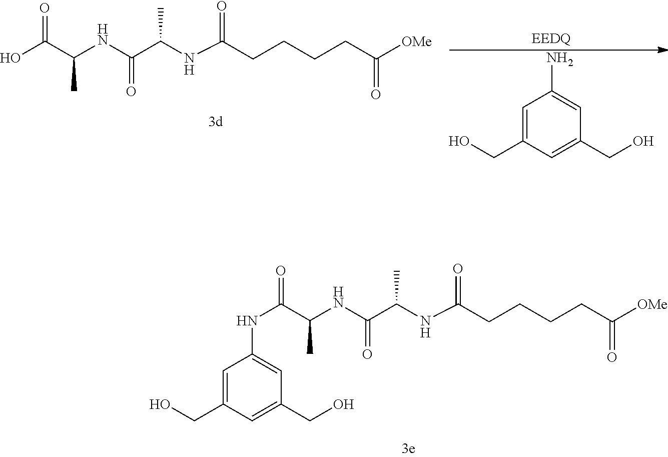 [SMILES: COC(=O)CCCCC(=O)N[C@@H](C)C(=O)N[C@@H](C)C(=O)O ; Nc1cc(CO)cc(CO)c1 ; COC(=O)CCCCC(=O)N[C@@H](C)C(=O)N[C@@H](C)C(=O)Nc1cc(CO)cc(CO)c1]

Compound 3d (5.6 g, 18.52 mmol) was dissolved in anhydrous dichloromethane (118 mL) and anhydrous methanol (58.8 mL). (5-amino-1,3-phenylene)dimethanol (2.70 g, 17.64 mmol) and EEDQ (8.72 g, 35.3 mmol) were then added, and the reaction was stirred at room temperature overnight. The solvent was stripped and ethyl acetate was added. The resulting slurry was filtered, washed with ethyl acetate, and dried under vacuum/N₂ to give compound 3e (2.79 g, 36% yield). 1H NMR (400 MHz, DMSO-d6): δ 9.82 (s, 1H), 8.05, (d, 1H, J=9.2 Hz), 8.01 (d, 1H, J=7.2 Hz), 7.46 (s, 2H), 6.95 (3, 1H), 5.21-5.12 (m, 2H), 4.47-4.42 (m, 4H), 4.40-4.33 (m, 1H), 4.33-4.24 (m, 1H), 3.58 (s, 3H), 2.33-2.26 (m, 2H), 2.16-2.09 (m, 2H), 1.54-1.46 (m, 4H), 1.30 (d, 3H, J=7.2 Hz), 1.22 (d, 3H, J=4.4 Hz).

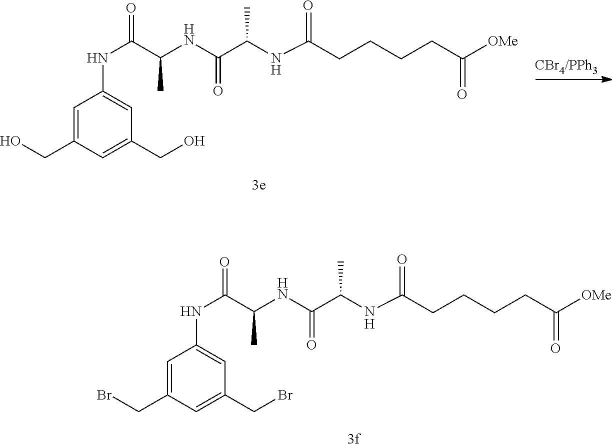 [SMILES: COC(=O)CCCCC(=O)N[C@@H](C)C(=O)N[C@@H](C)C(=O)Nc1cc(CO)cc(CO)c1 ; COC(=O)CCCCC(=O)N[C@@H](C)C(=O)N[C@@H](C)C(=O)Nc1cc(CBr)cc(CBr)c1]

Compound 3e (0.52 g, 1.189 mmol) and carbon tetrabromide (1.183 g, 3.57 mmol) were dissolved in anhydrous DMF (11.89 mL). Triphenylphosphine (0.935 g, 3.57 mmol) was then added, and the reaction was stirred under argon for 4 hours. The reaction mixture was diluted with DCM/MeOH (10:1) and washed with water and brine, dried over sodium sulfate, filtered, and concentrated. The crude material was purified by silica gel chromatography (DCM/MeOH) to give compound 3f (262 mg, 39% yield). 1H NMR (400 MHz, DMSO-d6): δ10.01 (s, 1H), 8.11 (d, 1H, J=6.8 Hz), 8.03 (d, 1H, J=6.8 Hz), 7.67 (s, 2H), 7.21 (s, 1H), 4.70-4.64 (m, 4H), 4.40-4.32 (m, 1H), 4.31-4.23 (m, 1H), 3.58 (s, 3H), 2.34-2.26 (m, 2H), 2.18-2.10 (m, 2H), 1.55-1.45 (m, 4H), 1.31 (d, 3H, J=7.2 Hz), 1.21 (d, 3H, J=7.2 Hz).

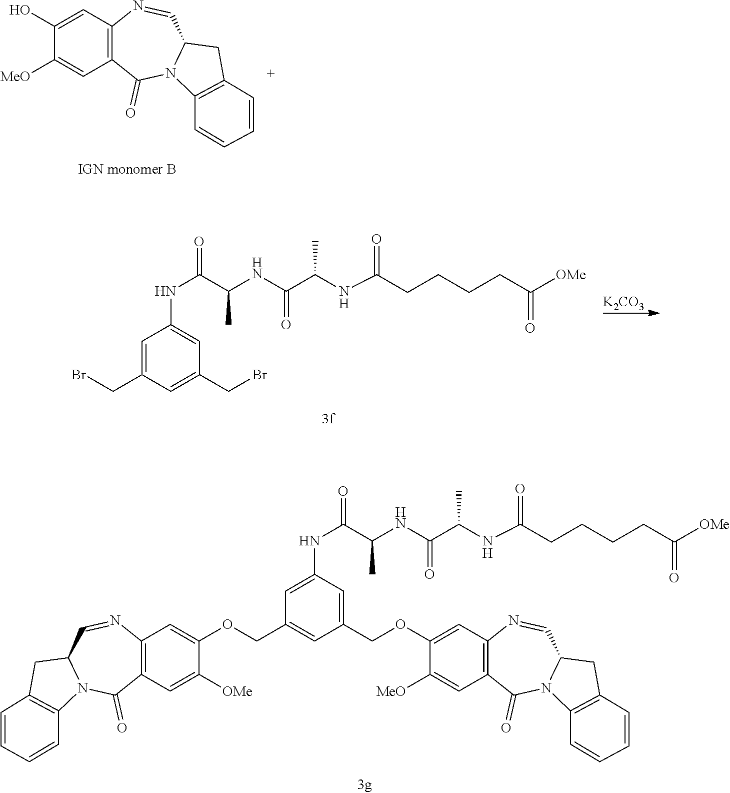

Dibromide compound 3f and IGN monomer B were dissolved in DMF. Potassium carbonate was added and was stirred at room temperature overnight. Water was added to the reaction mixture to precipitate the product. The slurry was stirred at room temperature and was then filtered and dried under vacuum/N₂. The crude material was purified by silica gel chromatography (dichloromethane/methanol) to give compound 3g (336 mg, 74% yield). LCMS=5.91 min (15 min method). MS (m/z): 990.6 (M+1)⁺.

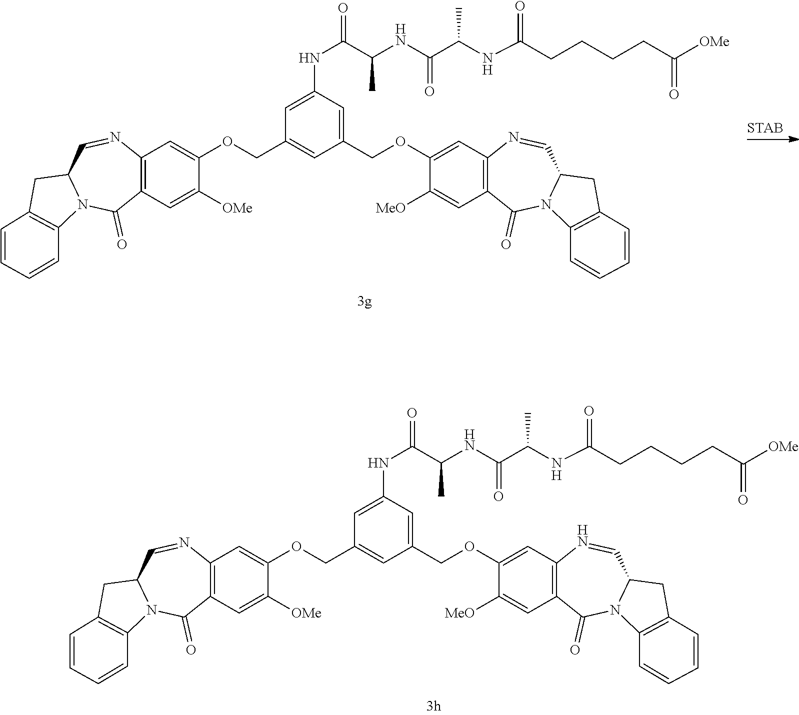

Diimine compound 3g was dissolved in 1,2-dichloroethane. NaBH(OAc)₃ (STAB) was added to the reaction mixture and was stirred at room temperature for 1 hour. The reaction was diluted with CH₂Cl₂ and was quenched with saturated NH₄Cl solution. The layers were separated and was washed with brine, dried over Na₂SO₄ and concentrated. The crude material was purified via RPHPLC (C-18 column, acetonitrile/water) to give compound 3h (85.5 mg, 25% yield). LCMS=6.64 min (15 min method). MS (m/z): 992.6 (M+1)⁺.

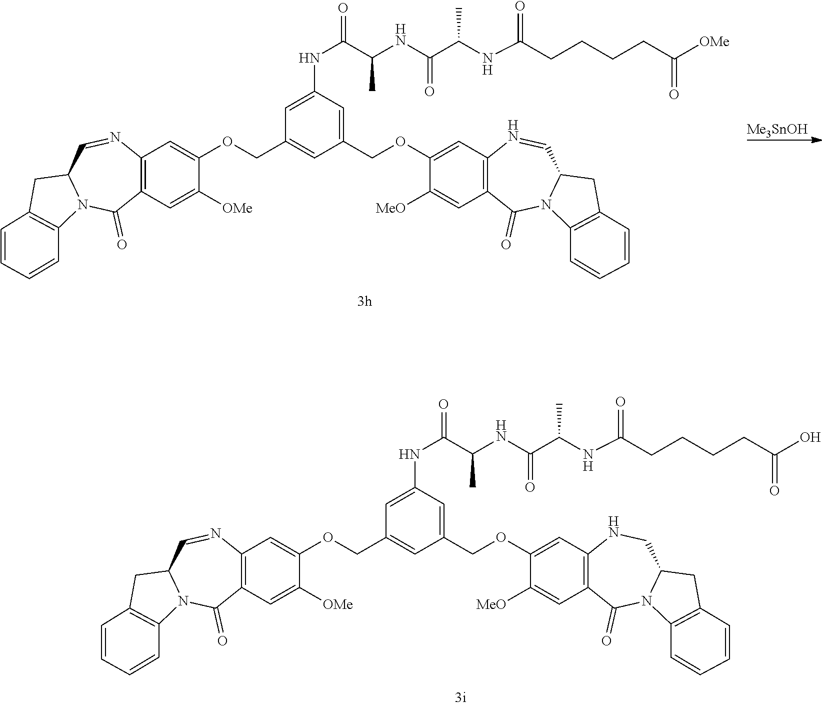

Compound 3h was dissolved in 1,2-dichloroethane. Trimethylstannanol was added to the reaction mixture and was heated at 80° C. overnight. The reaction mixture was then cooled to room temperature and diluted with water. The aqueous layer was acidified to pH˜4 with 1 M HCl. The mixture was extracted with CH₂Cl₂/MeOH. The combined organic layers were washed with brine, dried over Na₂SO₄, and concentrated. The crude material was passed through a silica plug to give compound 3i (48.8 mg, 80% yield). LCMS=5.89 min (15 min method). MS (m/z): 978.6 (M+1)⁺.

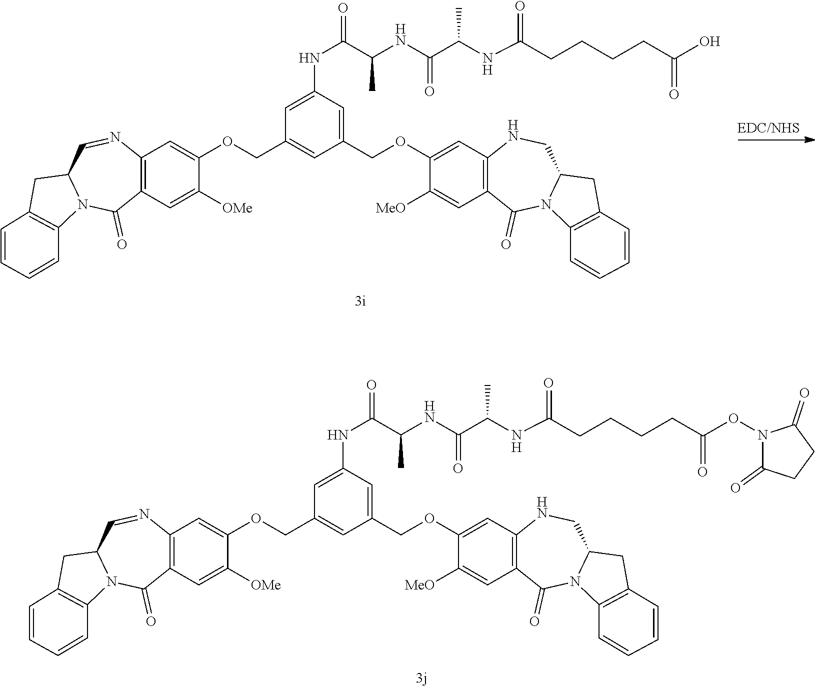 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)O)c1)N=C[C@@H]1Cc3ccccc3N1C2=O ; COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)ON3C(=O)CCC3=O)c1)N=C[C@@H]1Cc3ccccc3N1C2=O]

EDC.HCl was added to a stirred solution of acid compound 3i and N-hydroxysuccinamide in CH₂Cl₂ at room temperature. The reaction mixture was stirred for 2 hours. The reaction mixture was diluted with CH₂Cl₂ and washed with water and brine. The organic layer was dried over Na₂SO₄, filtered, and concentrated. The crude material was purified via RPHPLC (C-18 column, acetonitrile/water) to give 2,5-dioxopyrrolidin-1-yl 6-(((S)-1-(((S)-1-((3-((((S)-8-methoxy-6-oxo-11,12,12a,13-tetrahydro-6H-benzo[5,6][1,4]diazepino [1,2-a]indol-9-yl)oxy)methyl)-5-((((R)-8-methoxy-6-oxo-12a,13-dihydro-6H-benzo[5,6][1,4]diazepino[1,2-a]indol-9-yl)oxy)methyl) phenyl)amino)-1-oxopropan-2-yl)amino)-1-oxopropan-2-yl)amino)-6-oxohexanoate, compound 3j or CDA-3B (8.2 mg, 30% yield). LCMS=6.64 min (15 min method). MS (m/z): 1075.4 (M+1)⁺.

CDA-3A, the sulfonated form of CDA-3B, can be prepared by treating CDA-3B with NaHSO₃. See exemplary reaction conditions for converting CDA-2B to CDA-2A in Example 3 above.

### Example 5: Preparation of Antibody-Drug Conjugates

A. Preparation of hu5F9-CDA-1

i. Conjugation

Human 5F9 antibody was exchanged into 15 mM HEPES, pH 8.5 buffer prior to conjugation. Conjugates were then made using a 2-step reaction protocol. In step 1, sulfo-SPDB linker (see, e.g., paragraph [042], U.S. Pat. No. 8,236,319) was titrated with 5F9 antibody (representative molar excesses described in Table 2) in a 97/3 aqueous:organic ratio of 15 mM HEPES, pH 8.5 and dimethylacetamide (DMA) to a final antibody concentration of 4 mg/mL. This reaction mixture was incubated for 2 hours in a 25° C. water bath, and then purified as described below.

In step 2, 1.5 molar equivalents of CDA-1 over sulfo-SPDB were added to the antibody-linker mixture in a 85/15 aqueous:organic ratio of 15 mM HEPES, pH 8.5 and DMA. This reaction mixture was incubated for 4 hours in a 25° C. water bath before purification into formulation buffer (10 mM histidine, 50 mM sodium chloride, 8.5% sucrose, 0.01% Tween-20, 50 μM sodium bisulfite, pH 6.2).

TABLE 2

| | Molar Excess of | Conjugation Scale | Conjugation |
|---|---|---|---|
| Conjugate | sulfo-SPDB | (mg) | Yield |
| 5F9-CDA-1 | 7.0 | 50 | 35% |

ii. Purification

The 5F9-sulfo-SPDB reaction mixture was purified using Sephadex G-25 NAP columns equilibrated in 10 mM potassium phosphate, pH 7.9. The purified reaction mixture was filtered using a 0.22 μm PVDF syringe filter prior to linker-to-antibody ratio (LAR) analysis.

The 5F9-sulfo-SPDB-CDA-1 (5F9-CDA-1) conjugation reaction mixture was filtered through Sephadex G-25 gel filtration columns equilibrated with 20 mM histidine, 50 mM sodium chloride, 8.5% sucrose, 0.01% Tween-20, and 50 μM sodium bisulfite, pH 6.2. The purified conjugate was filtered using a 0.22 μm PVDF syringe filter and stored overnight at 4° C. The following day the sulfonated conjugate was re-filtered using 0.22 μm PVDF syringe filter before analysis.

B. Preparation of hu5F9-CDA-2

i. Conjugation

Human 5F9 antibody was exchanged into 15 mM HEPES, pH 8.5 buffer prior to conjugation. Conjugates were then made using a 2-step reaction protocol. In step 1, sulfo-SPDB linker was titrated with 5F9 antibody (representative molar excesses described in Table 3) in a 97/3 aqueous:organic ratio of 15 mM HEPES, pH 8.5 and DMA to a final antibody concentration of 4 mg/mL. This reaction mixture was incubated for 2 hours in a 25° C. water bath, and then purified as described below.

In step 2, 1.5 molar equivalents of CDA-2 over sulfo-SPDB were added to the antibody-linker mixture in a 85/15 aqueous:organic ratio of 15 mM HEPES, pH 8.5 and DMA. This reaction mixture was incubated for 4 hours in a 25° C. water bath before purification into formulation buffer (10 mM histidine, 50 mM sodium chloride, 8.5% sucrose, 0.01% Tween-20, 50 μM sodium bisulfite, pH 6.2).

TABLE 3

| | Molar Excess of | Conjugation Scale | Conjugation |
|---|---|---|---|
| Conjugate | sulfo-SPDB | (mg) | Yield |
| 5F9-CDA-2 | 8.0 | 20 | 23% |

ii. Purification

The 5F9-sulfo-SPDB reaction mixture was purified using Sephadex G-25 NAP columns equilibrated in 10 mM potassium phosphate, pH 7.9. The purified reaction mixture was filtered using a 0.22 μm PVDF syringe filter prior to LAR analysis.

The 5F9-sulfo-SPDB-CDA-2 (5F9-CDA-2) conjugation reaction mixture was filtered through Sephadex G-25 gel filtration columns equilibrated with 20 mM histidine, 50 mM sodium chloride, 8.5% sucrose, 0.01% Tween-20, and 50 μM sodium bisulfite, pH 6.2. The purified conjugate was filtered using a 0.22 μm PVDF syringe filter and stored overnight at 4° C. The following day the sulfonated conjugate was re-filtered using 0.22 μm PVDF syringe filter before analysis.

C. Preparation of hu5F9-CDA-3

i. Conjugation and Purification: Platform Protocol

Human 5F9 antibody was buffer exchanged into 15 mM HEPES, pH 8.5 prior to conjugation. 5F9-CDA-3 conjugates were then prepared using sulfonated form of CDA-3, CDA-3A. CDA-3A was initially sulfonated through incubation of CDA-3B with a 5-fold molar excess of sodium bisulfite and 50 mM succinate (pH 5.0) in a 90/10 organic:aqueous solution at ambient temperature for 3 hours followed by overnight incubation at 4° C. The conjugation reaction was then performed using 2.0 mg/mL of 5F9 antibody in 15 mM HEPES, pH 8.5 and the addition of CDA-3A at a specified molar excess based on the antibody (see Table 4 for representative conjugation). The conjugation reaction had a final 90/10 aqueous:organic composition of 15 mM HEPES, pH 8.5 and DMA, and was incubated in a water bath at 25° C. for 4 hours prior to purification into formulation buffer (10 mM histidine, 50 mM sodium chloride, 8.5% sucrose, 0.01% Tween-20, 50 μM sodium bisulfite, pH 6.2).

The 5F9-CDA-3 conjugation reaction mixture was purified using Sephadex G-25 NAP columns equilibrated with 10 mM histidine, 50 mM sodium chloride, 8.5% sucrose, 0.01% Tween-20, and 50 μM sodium bisulfite, pH 6.2. The purified conjugate was filtered using a 0.22 μm PVDF syringe filter and dialyzed overnight against fresh formulation buffer at 4° C., followed by dialysis at ambient temperature for 4 hours using fresh formulation buffer. The conjugate was re-filtered using a 0.22 μm PVDF syringe filter before analysis.

TABLE 4

| | Molar Excess of | Conjugation Scale | Conjugation |
|---|---|---|---|
| Conjugate | Sulfonated CDA-3 | (mg) | Yield |
| 5F9-CDA-3 | 5.4 | 220 | 24% |

ii. Conjugation and Purification: Optimized Protocol I

Various parameters including isotonic strength, conductivity, pH, reaction concentration, and molar equivalents of CDA-3 were explored to optimize the yield of desired 5F9-CDA-3 conjugate. An optimized protocol utilizing 75 mM EPPS, pH 8.0 buffer emerged from these studies. Similar to the standard platform protocol, 5F9-CDA-3 conjugates were made using sulfonated CDA-3A (prepared as described in the previous section). The optimized conjugation reaction was carried out using 2.0 mg/mL of 5F9 antibody in 75 mM EPPS, pH 8.0 and the addition of CDA-3A at a specified molar excess based on the antibody (see Table 5 for representative conjugation). The conjugation reaction had a final 90/10 aqueous:organic composition of 75 mM EPPS, pH 8.0 and DMA, and was incubated in a water bath at 25° C. for 4 hours prior to purification into formulation buffer (10 mM histidine, 50 mM sodium chloride, 8.5% sucrose, 0.01% Tween-20, 50 μM sodium bisulfite, pH 6.2).

The 5F9-CDA-3 conjugation reaction mixture was purified using Sephadex G-25 NAP columns equilibrated with 10 mM histidine, 50 mM sodium chloride, 8.5% sucrose, 0.01% Tween-20, and 50 μM sodium bisulfite, pH 6.2. The purified conjugate was filtered using a 0.22 μm PVDF syringe filter and dialyzed overnight against fresh formulation buffer at 4° C., followed by dialysis at ambient temperature for 4 hours using fresh formulation buffer. The conjugate was re-filtered using a 0.22 μm PVDF syringe filter before analysis.

TABLE 5

| | Molar Excess of | Conjugation Scale | Conjugation |
|---|---|---|---|
| Conjugate | Sulfonated CDA-3 | (mg) | Yield |
| 5F9-CDA-3 | 4.0 | 60 | 64% |

iii. Conjugation and Purification: Optimized Protocol II

### Optimized Sulfonation

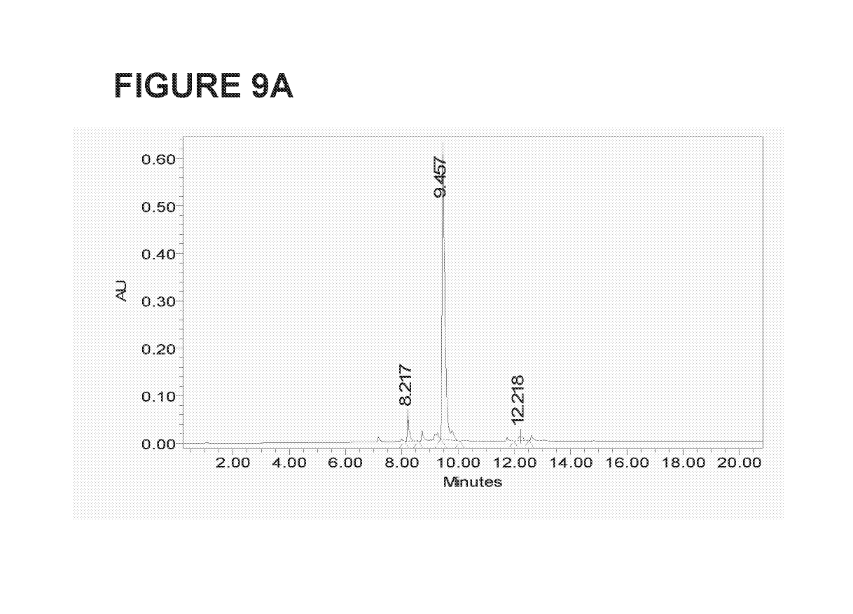

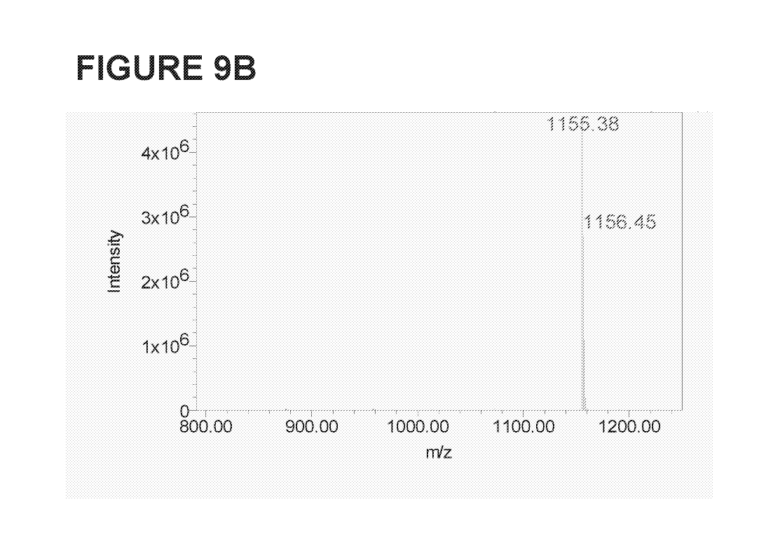

CDA-3B was sulfonated as follows to generate CDA-3A. To 3.75 mL of a 50 mM sodium succinate, pH 3.3, DMA in the amount of 6.11 mL was added. After mixing and equilibration to 10° C. in a water bath, 1.39 mL of a 21.5 mM CDA-3B stock solution in DMA (30.0 μmol CDA-3) was added and mixed. Following this addition, 3.75 mL of a 20 mM aqueous sodium bisulfite solution (2.5 equivalents, 75 μmol) was introduced into the reaction. After mixing, the reaction was allowed to proceed at 10° C. for 15.5 hours and was used immediately in the next step without purification. Liquid chromatography (reverse phase) analysis of the reaction mixture indicated 92.4% conversion to CDA-3A with 2.4% remaining unreacted CDA-3B was shown in FIG. 9A. The peak identity of CDA-3A was confirmed by LC/MS as shown in FIG. 9B.

### Post Conjugation Quench

In order to determine a condition wherein an increase in ionic strength post conjugation results in a decrease in the formation of the high molecular weight (HMW) species, the following optimization was performed. 5F9 antibody (2 mg/mL) was conjugated to 3.8 molar equivalents of CDA-3A at 22° C. for 80-90 minutes. The final composition of the conjugation reaction comprised of 130 mM EPPS, pH 8.7 with 15% DMA by volume. Immediately upon completion of the conjugation reaction, aliquots were diluted with the indicated volume of the quench solution as detailed in Table 6. Changes in the percent HMW species were monitored for the indicated time upon holding at 22° C. Based on this finding, 1.4-1.6-fold dilutions using 750 mM EPPS, and 1.4-1.6-fold dilutions using 750 mM EPPS/150 mM histidine hydrochloride were selected. In the following conjugation example, 1.5-fold dilution using 750 mM EPPS/150 mM histidine hydrochloride was used. Table 6 depicts the effects of quench solutions on the stability of crude 5F9-CDA-3 conjugate. Crude 5f9-CDA-3 conjugate was incubated with different quench solutions for the specified amount of time and the changes in the percent molecular weight species were determined by size exclusion chromatography.

TABLE 6

| | | | | Quench | Time | Δ | |
|---|---|---|---|---|---|---|---|
| | | | Crude | solution | post | % HMW | |
| | Quench | Fold | reaction | vol. | mixing | (initial- | Visible |
| Experiment | Solution | dilution | (mL) | (mL) | (min) | final)* | precipitation |
| 1 | None (control | NA | 0.5 | NA | 720 | 2.0 | no |
| | for | | | | | | |
| | experiments 2- | | | | | | |
| | 6) | | | | | | |
| 2 | 6.7% w/v % | 2.0 | 0.5 | 0.5 | 760 | NA | yes |
| | sucrose, 50uM | | | | | | |
| | sodium | | | | | | |
| | bisulfite | | | | | | |
| 3 | 13.4% w/v % | 2.0 | 0.5 | 0.5 | 800 | NA | yes |
| | sucrose, 50uM | | | | | | |
| | sodium | | | | | | |
| | bisulfite | | | | | | |
| 4 | 20% w/v % | 1.5 | 0.5 | 0.25 | 840 | NA | yes |
| | sucrose, | | | | | | |
| | 100uM sodium | | | | | | |
| | bisulfite | | | | | | |
| 5 | 50 mM | 2.0 | 0.5 | 0.5 | 880 | NA | yes |
| | histidine, | | | | | | |
| | 6.7 w/v % | | | | | | |
| | sucrose, 50 | | | | | | |
| | uM sodium | | | | | | |
| | bisulfite, pH | | | | | | |
| | 5.5 | | | | | | |
| 6 | 130 mM EPPS, | 2.0 | 0.5 | 0.5 | 920 | NA | yes |
| | 50 uM sodium | | | | | | |
| | bisulfite, pH | | | | | | |
| | 8.7 | | | | | | |
| 7 | None (control | NA | 0.5 | NA | 850 | 2.4 | no |
| | for | | | | | | |
| | experiments 8- | | | | | | |
| | 13) | | | | | | |
| 8 | 400 mM | 1.2 | 0.5 | 0.075 | 890 | 4.2 | no |
| | succinic acid, | | | | | | |
| | 50 uM sodium | | | | | | |
| | bisulfite | | | | | | |
| 9 | 60 mM | 2.0 | 0.5 | 0.5 | 930 | 2.3 | no |
| | succinic acid, | | | | | | |
| | 50 uM sodium | | | | | | |
| | bisulfite | | | | | | |
| 10 | 60 mM | 2.0 | 0.5 | 0.5 | 970 | 4.2 | no |
| | succinic acid, | | | | | | |
| | 13.4% sucrose, | | | | | | |
| | 50 uM sodium | | | | | | |
| | bisulfite | | | | | | |
| 11 | 300 mM EPPS, | 2.0 | 0.5 | 0.5 | 1010 | 1.0 | no |
| | 50 uM sodium | | | | | | |
| | bisulfite, pH | | | | | | |
| | 8.7 | | | | | | |
| 12 | 500 mM EPPS, | 2.0 | 0.5 | 0.5 | 1050 | 0.8 | no |
| | 50 uM sodium | | | | | | |
| | bisulfite, pH | | | | | | |
| | 8.7 | | | | | | |
| 13 | 700 mM EPPS, | 2.0 | 0.5 | 0.5 | 1090 | 0.6 | no |
| | 50 uM sodium | | | | | | |
| | bisulfite, pH | | | | | | |
| | 8.7 | | | | | | |
| 14 | None (control | NA | 1.0 | NA | 880 | 2.9 | no |
| | experiments | | | | | | |
| | for 15-20) | | | | | | |
| 15 | 600 mM | 1.4 | 1.0 | 0.4 | 600 | 2.8 | no |
| | histidine | | | | | | |
| | hydrochloride | | | | | | |
| 16 | 600 mM | 1.5 | 1.0 | 0.5 | 640 | 3.2 | no |
| | histidine | | | | | | |
| | hydrochloride | | | | | | |
| 17 | 600 mM | 1.6 | 1.0 | 0.6 | 680 | 3.6 | no |
| | histidine | | | | | | |
| | hydrochloride | | | | | | |
| 18 | 600 mM | 1.4 | 1.0 | 0.4 | 720 | 2.7 | no |
| | histidine | | | | | | |
| | hydrochloride, | | | | | | |
| | 20 w/v % | | | | | | |
| | sucrose | | | | | | |
| 19 | 600 mM | 1.5 | 1.0 | 0.5 | 760 | 2.9 | no |
| | histidine | | | | | | |
| | hydrochloride, | | | | | | |
| | 20 w/v % | | | | | | |
| | sucrose | | | | | | |
| 20 | 600 mM | 1.6 | 1.0 | 0.6 | 800 | 2.9 | no |
| | histidine | | | | | | |
| | hydrochloride, | | | | | | |
| | 20 w/v % | | | | | | |
| | sucrose | | | | | | |
| 21 | None (control | NA | 1.0 | NA | 840 | 2.2 | no |
| | experiments | | | | | | |
| | for 22-27) | | | | | | |
| 22 | 750 mM EPPS | 1.4 | 1.0 | 0.4 | 600 | 0.4 | no |
| 23 | 750 mM EPPS | 1.5 | 1.0 | 0.5 | 640 | 0.5 | no |
| 24 | 750 mM EPPS | 1.6 | 1.0 | 0.6 | 680 | 0.4 | no |
| 25 | 750 mM EPPS, | 1.4 | 1.0 | 0.4 | 720 | 0.5 | no |
| | 150 mM | | | | | | |
| | histidine | | | | | | |
| | hydrochloride | | | | | | |
| 26 | 751 mM EPPS, | 1.5 | 1.0 | 0.5 | 760 | 0.5 | no |
| | 150 mM | | | | | | |
| | histidine | | | | | | |
| | hydrochloride | | | | | | |
| 27 | 752 mM EPPS, | 1.6 | 1.0 | 0.6 | 800 | 0.5 | no |
| | 150 mM | | | | | | |
| | histidine | | | | | | |
| | hydrochloride | | | | | | |

*Calculated by subtracting the % HMW of the appropriate control at t = 0 min from the experiment % HMW at the time indicated in the table.

### Optimized Conjugation and Purification

In a 1 L jacketed glass reactor equipped with an overhead stirrer containing 325 mL of 130 mM EPPS, pH 8.7, 68.6 mL of DMA was added. Following mixing and equilibration of the solution to 22° C., 100 mL of a 10.0 mg/mL solution of 5F9 antibody in 130 mM EPPS, pH 8.7 was introduced into the reactor and allowed to mix for 15 minutes. Subsequently 12.8 mL of the 2 mM CDA-3A solution (25.5 μmol, 3.7 equivalents of 5F9 antibody; prepared using the optimized sulfonation protocol described previously) was introduced into the reaction solution. After stirring for 60 min at 22° C., 250 mL of an aqueous solution containing 150 mM histidine hydrochloride and 750 mM EPPS was transferred into the reaction vessel. After mixing thoroughly, this material was filtered through a Millipore Optiscale 47 Express SHC 0.5/0.2 μM filter. The crude reaction mixture was then concentrated by ultrafiltration with a TangenX 0.02 m² HyStream 30 kD Sius LSN TFF cassette to a calculated bulk protein concentration of 2.5 mg/mL. Following the concentration step, the solution was diafiltered against 4.8 L of a 50 mM histidine, 6.7 w/v (weight/volume) % sucrose, 0.1 v/v (volume/volume) % polysorbate-80, 50 μM sodium bisulfite, pH 5.5 buffer. After diafiltration, the retentate solution was filtered with a Millipore Optiscale 47 Express SHC 0.5/0.2 μM filter. Following storage at 2-8° C. for 2 days, the solution was diluted to 1.0 mg/mL conjugate by addition of the necessary volume of additional 50 mM histidine, 6.7 w/v % sucrose, 0.1 v/v % polysorbate-80, 50 μM sodium bisulfite, pH 5.5 buffer. This solution was then filtered through a Millipore Optiscale 47 Durapore 0.22 μM filter giving 818 mL of 1.0 mg/mL conjugate. The measured DAR of the final conjugate is 2.6 by UV/vis with 97.4% monomer and 2.5% HMW by SEC. The final yield of the product was 82%.

D. Preparation of 5F9-PVAdG-CDA-3

The protocol described in the previous section utilizing 75 mM EPPS, pH 8.0 buffer was used to prepare the 5F9-PVAdG-CDA-3 conjugate. The 5F9-PVAdG antibody contains amino acid substitutions that replace ELLG in the heavy chain of IgG1 (SEQ ID NO:9), which are important for binding FcγRIIIb, with PVA, the highly conserved amino acids in IgG2 at the analogous location (Vidarsson et al., IgG subclasses and allotypes: from structure to effector functions, Frontiers in Immunology, 5(520): 1-17(2014)).

The conjugation reaction was carried out using 5F9 PVAdG antibody at 2.0 mg/mL in 75 mM EPPS, pH 8.0 with the addition of sulfonated CDA-3A at a specified molar excess based on the antibody (see Table 6 for representative conjugation). The conjugation reaction had a final 90/10 aqueous:organic composition of 75 mM EPPS, pH 8.0 and DMA, and was incubated in a water bath at 25° C. for 4 hours prior to purification into formulation buffer (10 mM histidine, 50 mM sodium chloride, 8.5% sucrose, 0.01% Tween-20, 50 μM sodium bisulfite, pH 6.2).

The 5F9-PVAdG-CDA-3 conjugation reaction mixture was purified using Sephadex G-25 HiPrep columns equilibrated with 10 mM histidine, 50 mM sodium chloride, 8.5% sucrose, 0.01% Tween-20, 50 μM sodium bisulfite, pH 6.2. The purified conjugate was filtered using a 0.22 μm PVDF syringe filter before analysis.

TABLE 7

| | Molar Excess of | Conjugation Scale | Conjugation | CDA-3/Antibody | Monomeric |
|---|---|---|---|---|---|
| Conjugate | Sulfonated CDA-3 | (mg) | Yield | Ratio | Conjugate |
| 5F9-PVAdG-CDA-3 | 3.9 | 98 | 78% | 2.85 | 99% |

### Example 6: Analysis of Antibody-Drug Conjugates

A. Determination of Linker-To-Antibody Ratio (LAR)

The concentration of antibody in purified 5F9-sulfo-SPDB was determined by ultra-violet/visible-range spectroscopy (UV/Vis) using absorbance values at 280 nm and the extinction co-efficient for the 5F9 antibody (ε=224,000 M⁻¹; Table 8). The concentration of sulfo-SPDB linker was measured assuming a 1:1 ratio of linked molecule per released thiopyridine upon treatment with dithiothreitol (DTT) in pH 7.5 buffer, followed by a UV/Vis assay at 343 nm (ε=8,080 M⁻¹). The ratio of molar concentrations of linker to antibody is reported as the LAR value.

B. Determination of Drug-To-Antibody Ratio (DAR)

The concentration of 5F9 antibody and CDA in purified conjugate samples was determined by UV/Vis using absorbance values at 280 nm and 330 nm. Since both the antibody and the CDAs absorb at 280 nm, a binomial equation was required to consider the portion of total signal attributed to each moiety. Only CDAs absorb at 330 nm, so the concentration at that wavelength can be attributed solely to the effector molecule. The extinction co-efficient values of conjugated moiety are listed in Table 7.

The antibody and CDA components were quantified using the following algebraic expressions, which account for the contribution of each constituent at each wavelength:

C_{CDA}=A₃₃₀/ε_{330 nm IGN}

C_{Ab}=(A₂₈₀−(ε_{280 nm IGN}/ε_{330 nm IGN})×A₃₃₀)/ε_{280 nm Ab}

Aₓ is the absorbance value at X nm wavelength, whereas C_{Ab} is the molar concentration of antibody (i.e., 5F9) and C_{CDA} is the molar concentration of CDA. The ratio of CDA:Ab (DAR) was calculated as a ratio of the above molar concentrations. The mg/mL (g/L) concentrations of 5F9 and CDA were calculated using the molecular weights listed in Table 8.

TABLE 8

| | MW of | | |
|---|---|---|---|
| | conjugated | | |
| | moiety* | ε₂₈₀ | ε₃₃₀ |
| Moiety | (g/mol) | (M⁻¹cm⁻¹) | (M⁻¹cm⁻¹) |
| 5F9 antibody | 144,898 | 224,000 | n/a |
| CDA-1 | 942 | 30,115 | 15,484 |
| CDA-2 | 838 | 30,115 | 15,484 |
| CDA-3 | 961 | 30,115 | 15,484 |
| CDA-3* | 961 | 34,150 | 16,270 |

Recalculated using alternate protocol for use in Conjugation and Purification: Optimized Protocol II.

C. Determining the Percent of Monomeric Conjugate

The percentage of monomeric conjugate in purified 5F9-CDA samples was determined via HPLC analysis using size-exclusion chromatography (SEC). Approximately 10-100 μg of 5F9-CDA conjugate was injected onto an HPLC instrument with an attached SEC column (TSK GEL G3000SWxl 5 μm, 7.8 mm×30 cm, Part No. 08541; recommended guard column TSK GEL, 4 cm, Part No. 08543, TOSOH Biosciences, King of Prussia, Pa.), and run at 0.5 mL per minute with an isocratic mobile phase of 400 mM sodium perchlorate, 50 mM sodium phosphate, 5% isopropanol. Absorbance signal was collected for 30 min at 280 nm and 330 nm wavelengths.

5F9 antibody monomer typically eluted at ˜17 min, while 5F9-CDA conjugate monomer often eluted as a doublet with peaks at ˜17 and ˜19 min. High molecular weight species (HMW, e.g., dimer, aggregate) and low molecular weight species (LMW, e.g., fragment) typically eluted at ˜12 and ˜24 min, respectively.

The % monomeric antibody (or conjugate) was calculated from the 280 nm peak area of the 17 min peak (or the 17/19 doublet), and compared to the area of all of the protein peaks combined. The DAR on the monomer peak was also determined by substituting the peak areas of 280 nm and 330 nm signals into the A₂₈₀ and A₃₃₀ spaces in the C_{CDA} and C_{Ab} equations shown in the above section, and then dividing C_{CDA}/C_{Ab}.

D. Determining the Percent of Unconjugated CDA

The amount of unconjugated CDA (“free drug”) present in purified 5F9-CDA samples was determined via UPLC analysis using tandem SEC and C-18 reverse-phase columns (“dual-column”). Two Waters Acquity UPLC Protein BEH SEC columns (1.7 μm, 4.6×30 mm, Part No. 186005793, Waters Corporation, Milford, Mass.) were connected in series to separate the intact 5F9-CDA conjugate from free drug, which was then channeled to a Waters Cortecs UPLC C-18 column (2.1×50 mm, Part No. 186007093) to separate and quantify free CDA species. The 5F9-CDA conjugate was prepared by diluting with acetonitrile (ACN) to 20% (v/v) ACN, injected onto the column series (25 μL), and run according to the gradient listed in Table 9:

TABLE 9

| Time (min) | Flow (mL/min) | % A | % B |
|---|---|---|---|
| 0.0 | 0.35 | 70 | 30 |
| 1.0 | 0.35 | 70 | 30 |
| 8.0 | 0.35 | 20 | 80 |
| 9.0 | 0.35 | 5 | 95 |
| 10.0 | 0.35 | 5 | 95 |
| 10.1 | 0.35 | 70 | 30 |
| 11.0 | 0.35 | 70 | 30 |
| 12.0 | 0.35 | 70 | 30 |
| 13.5 | 0.35 | 70 | 30 |
| 14.0 | 0.35 | 95 | 5 |
| 20.0 | 0.35 | 95 | 5 |
| 21.0 | 0.0 | 95 | 5 |

Table 9: Flow rate = 0.35 ml/min;
run time = 12.5 minutes;
C-18 column temperature = 30° C.;
mobile phases = A: 0.1% (v/v) TFA in water, B: 0.1% (v/v) TFA in ACN

The column was diverted from in-line SEC to C-18 at 2.2 min and back to in-line SEC at 14.0 min. Signal was collected at 265 nm. Using a standard curve derived from CDA-1 or CDA-3, the amount of free drug present in the sample was calculated from peaks found in the 2.2-14.0 minute window, using the following formulas:

ng_{free CDA-1}=(AUC_{265 nm}+353)/5406

ng_{free CDA-3}=(AUC_{265 nm}+11805)/4888

% free CDA=ng_{free CDA}/ng_{injected}

### Example 7: Characterization of Antibody-Drug Conjugates

### Cell Lines

The cell lines used for functional assays were cell pairs of GCC-transfected and vector control human embryonic kidney (HEK) 293 cells. HEK293 cells were transfected with myc-tagged, full-length GCC under control of the CMV promoter, or with an empty vector (pN8mycSV40), and selected in blasticidin. HEK293-GCC#2 clones demonstrated the highest GCC expression. GCC expression in HEK293-GCC#2 cells was further analysed using whole cell binding assays with radiolabeled ligand (ST-toxin), and quantitation of GCC receptor levels suggested that HEK293-GCC#2 cells express more GCC than other GCC-expressing cell lines (e.g., GCC-transfected human colorectal adenocarcinoma HT-29 cells and T84 human colonic adenocarcinoma cells)

A. Cell Binding/Affinity Assays

To determine each antibody-drug conjugate's ability to bind GCC-expressing cells, 5F9-CDA conjugates were evaluated by an indirect immune-fluorescence assay using flow cytometry. HEK293-GCC#2 and vector control cells were grown in standard cell culture medium supplemented with 10% fetal bovine serum (FBS). Cells were non-enzymatically dislodged from the plate surface using Versene (ThermoFisher Scientific, Washington, D.C.; Catalog No. 15040-066), centrifuged for 5 min at 1200 rpm in a sterile tube containing FBS, and washed in 3% FBS/phosphate buffered saline (PBS) without Ca²⁺ or Mg²⁺. This centrifugation-wash step was repeated once more before cells were re-suspended at a concentration of 5×10⁶ cells/mL in 3% FBS/PBS, and added to the experimental wells of a V-bottom 96-well plate in 100 μL aliquots (˜500,000 cells). The plate was centrifuged for 5 min at 1200 rpm.

Following centrifugation, supernatant was removed from each well and replaced with 50 μL of primary antibody-drug conjugate solution. Solutions of 5F9-CDA-1, 5F9-CDA-2, and 5F9-CDA-3 were each prepared at a final concentration of 1 μg/mL. The 96-well dish was covered and incubated at 4° C. (on ice) for 1 hour before solutions were removed from the wells and the cells were washed twice in 100 μL of 3% FBS/PBS (without Ca²⁺ or Mg²⁺).

Goat F(ab′)2 anti-human IgG, mouse ads-PE (SouthernBiotech, Birmingham, Ala.; Catalog No. 2043-09) secondary antibody was diluted 1:200, as suggested by the manufacturer. After completion of the second wash, 50 μL of secondary antibody solution was added to each experimental well, and the covered 96-well plate was placed at 4° C. (on ice) for 1 hour. The plate was then centrifuged, and the supernatant was replaced with 100 μL of 3% FBS/PBS (without Ca²⁺ or Mg²⁺). This centrifugation-wash step was repeated for a total of two cycles. The cells were finally reconstituted in 200 μL of PBS (without Ca²⁺ or Mg²⁺) and loaded onto the BD FACS Canto flow cytometer (BD Biosciences, Franklin Lakes, N.J.). Data were analyzed using FACS II Canto system software and the appropriate filter settings.

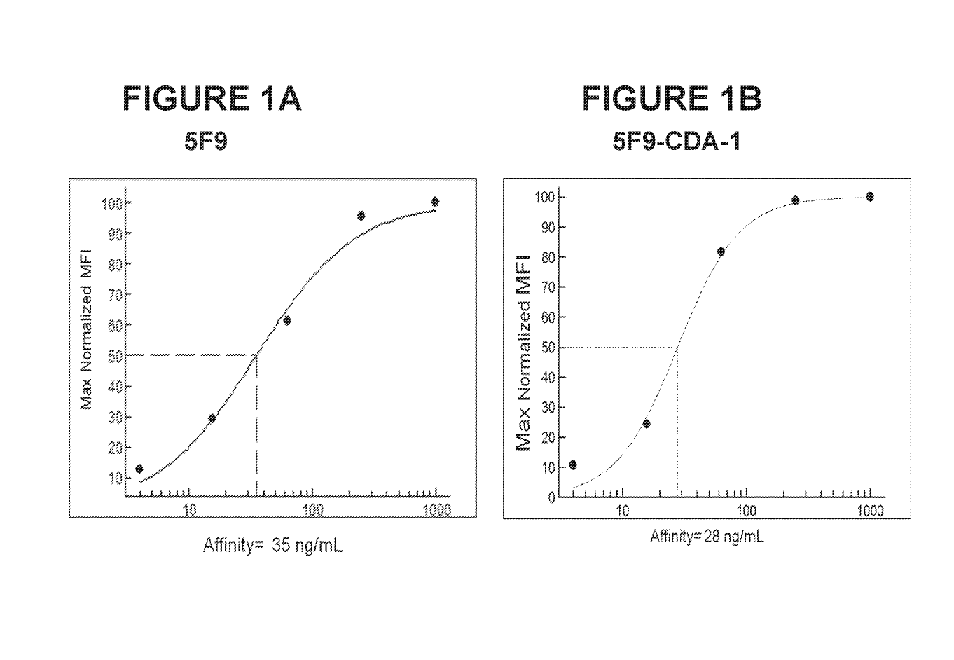

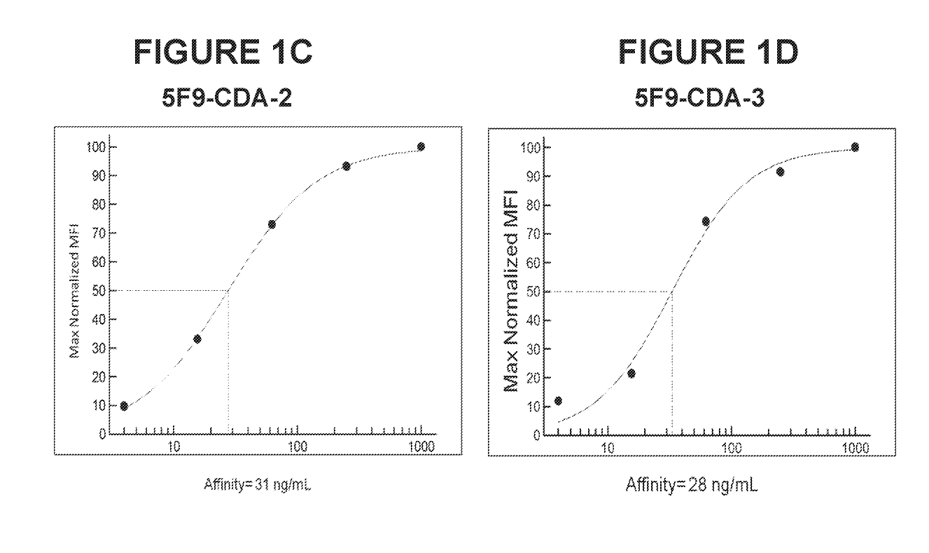

Conjugation of a CDA to an antibody molecule could alter the affinity of such antibody for its target antigen, or to disrupt such antibody's cell binding to its antigen. FIG. 1 demonstrates that CDA conjugation does not affect or reduce binding of the 5F9 antibody to GCC. Affinity values are comparable between unconjugated 5F9 (FIG. 1A), and the 5F9-CDA conjugates of the invention (FIGS. 1B-1D). Table 9 demonstrates that CDA conjugation to 5F9 in the 5F9-CDA conjugates does not affect or reduce binding of the antibody molecule to GCC.

TABLE 10

| Antibody or | |
|---|---|
| Antibody-Drug Conjugates | Affinity (ng/mL) |
| 5F9 | 35 |
| 5F9-CDA-1 | 28 |
| 5F9-CDA-2 | 31 |
| 5F9-CDA-3 | 28 |

B. Cytotoxicity/Potency Assays

To measure each 5F9-CDA conjugate's ability to kill GCC-expressing cells, cytotoxicity assays were performed. In this assay, GCC-expressing HEK293-GCC#2 cells and vector control cells were seeded at a density of 2×10³ per well into 96-well deep well plates in triplicate. Serial dilutions of the 5F9-CDAs were immediately added to the seeded wells, and plate was incubated at 37° C. for 96 hours. Following incubation, cell viability was evaluated using the CellTiter-Glo® Luminescent assay (Promega, Madison, Wis.), as recommended by the manufacturer. Viability was normalized to untreated control cells, and error was calculated as the standard error of the mean (SEM).

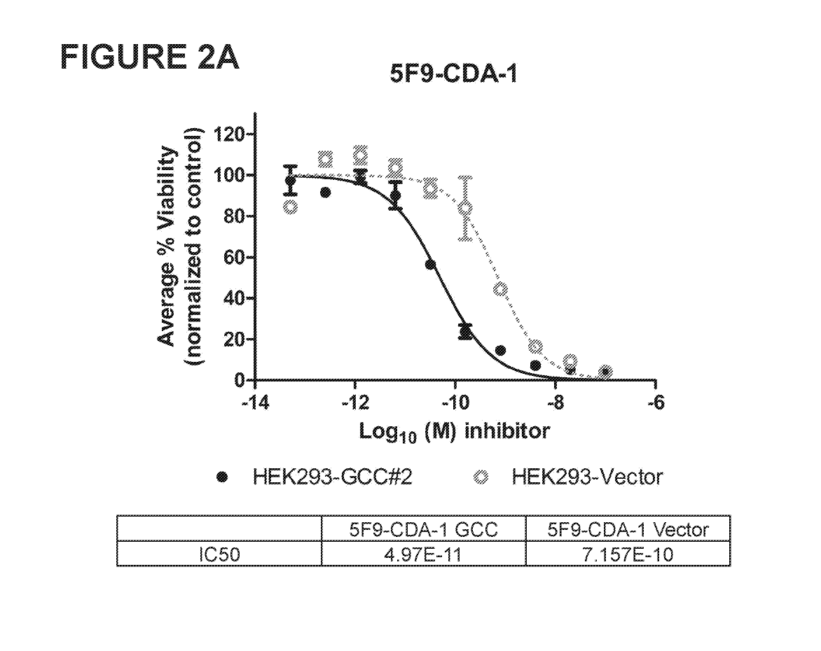

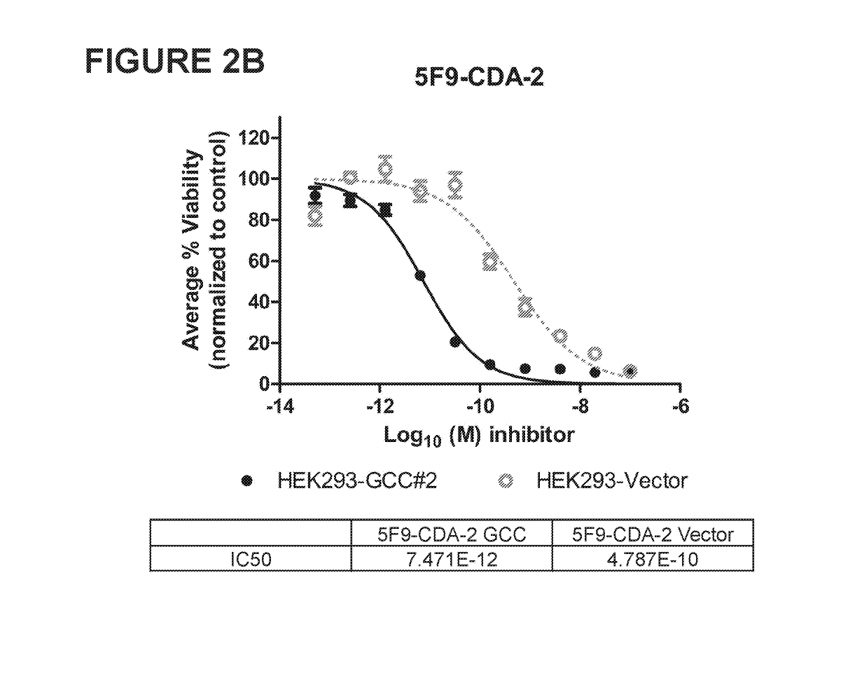

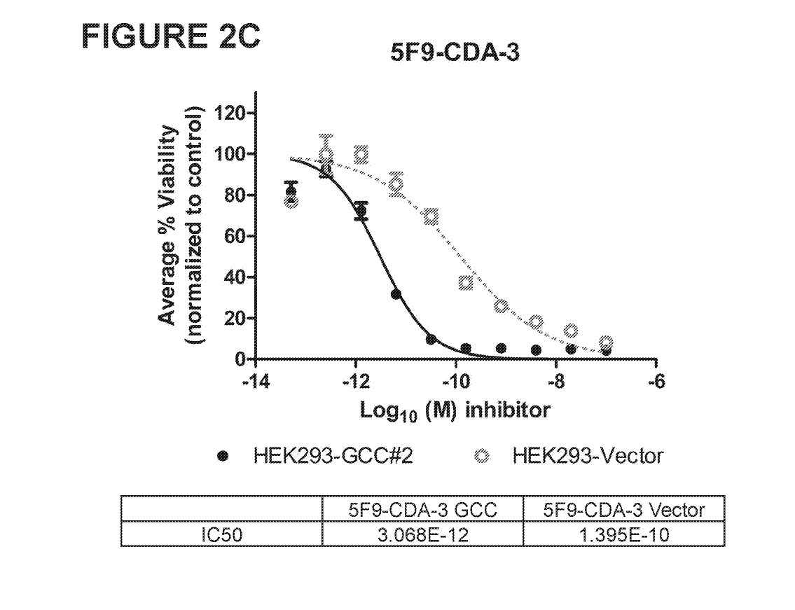

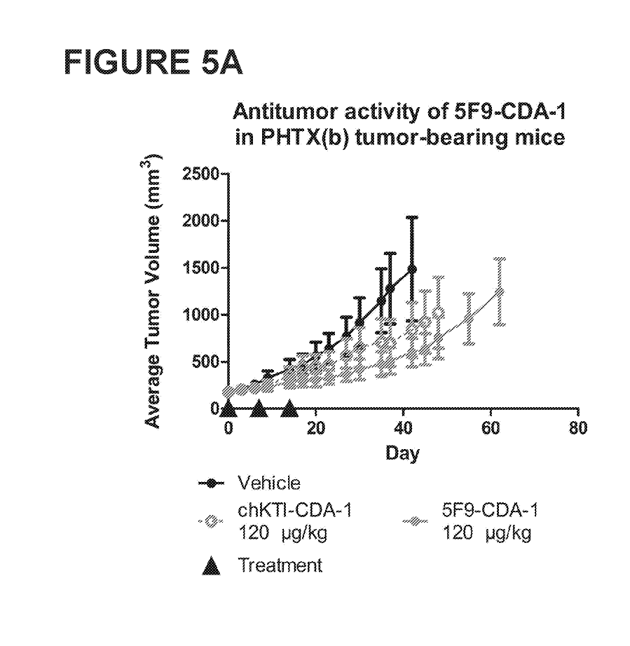

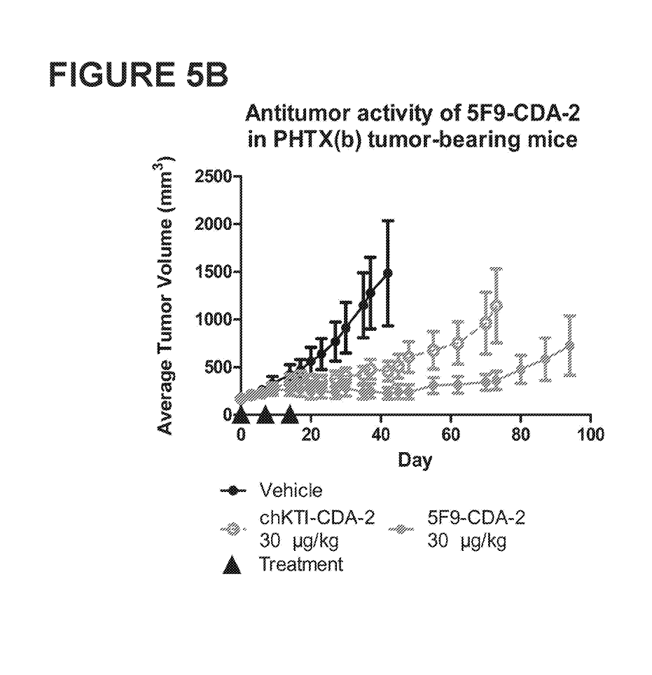

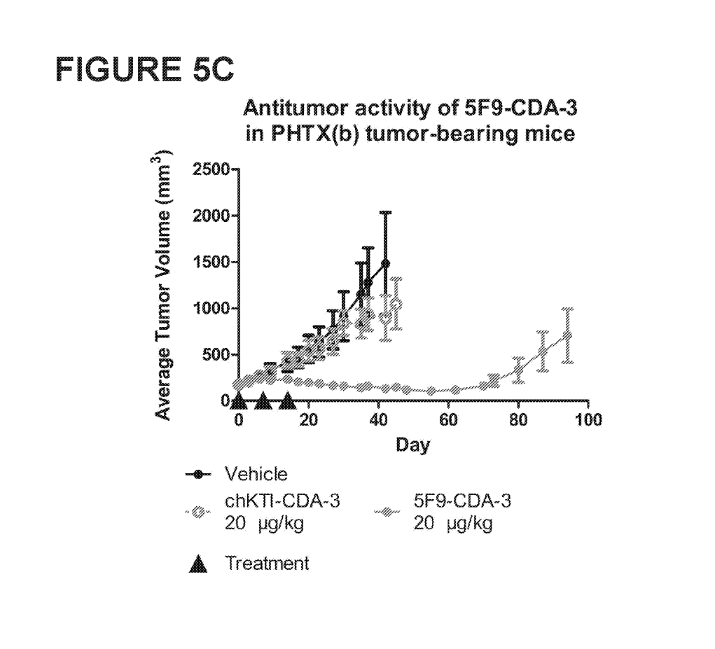

The relative potency of 5F9-CDA conjugates on HEK293-GCC#2 cells is shown in FIG. 2. 5F9-CDA-2 (FIG. 2B) and 5F9-CDA-3 (FIG. 2C) are more potent antibody-drug conjugates than 5F9-CDA-1 (FIG. 2A). See Table 11. These assays also demonstrate that the antibody-drug conjugates of the invention specifically target and kill GCC-expressing cells, and have significantly reduced cytotoxicity in cells that do not express GCC antigen.

TABLE 11

| Antibody or | |
|---|---|
| Antibody-Drug Conjugates | IC₅₀ (pM) |
| 5F9-CDA-1 | 49.7 |
| 5F9-CDA-2 | 7.47 |
| 5F9-CDA-3 | 3.07 |

C. Internalization Assays

Internalization of the anti-GCC antibody molecule was tested in both GCC-expressing HEK293-GCC#2 cells and vector control cells using immunofluorescence microscopy. Cells were grown on coverslips and placed on ice for 10 min prior to incubation with the 5F9 antibody (10 μg/mL) in cold culture medium for 20 min on ice. Antibody-containing medium was then replaced with fresh culture medium, and the cells were either incubated at 37° C. for 2-3 hours or maintained at 4° C. (on ice). After one rinse in PBS and a brief fixation in 4% paraformaldehyde at room temperature, cells were permeabilized for 15 min in 5% TRITON X-100. Localization of the 5F9 antibody was determined with a fluorescently-labeled anti-IgG antibody using laser scanning confocal microscopy. The 5F9 antibody localized to the cell surface of GCC-expressing cells when on ice, whereas cells incubated at 37° C. showed punctuate staining within the cell membrane, indicative of internalization. No internalization was detected in vector control cells.

### Example 8: In Vivo Evaluation

A. Efficacy of ADCs in Tumor Models

In vivo efficacy of the 5F9-CDA conjugates was evaluated in mouse xenograft models.

For all efficacy studies, female CB-17 SCID mice (6-7 weeks of age) were subcutaneously inoculated over the flank with 5×10⁶ HEK293-GCC#2 cells, or 6-7 week old nude mice were inoculated with 2 mm×3 mm tumor fragments of human primary tumors (PHTX) from patients (a), (b), and (c), in Dulbecco's Modified Eagle Medium (DMEM) without 10% FBS serially transplanted onto the flank. Animals were randomized into treatment groups when mean tumor volume reached approximately 200 mm³. Treatment groups (n=5 per group) included a control group dosed with the appropriate vehicle, a control group dosed with chKTI-CDA, or experimental groups dosed with 5F9-CDA conjugates of the invention.

Chimeric KTI (chKTI) antibody is a murine/human chimeric antibody derived from the ATCC hybridoma HB-9515, described in U.S. Pat. No. 4,959,310; Brandon et al., J. Food Sci. 53:97-101 (1988); Brandon et al., J. Agric. Food Chem. 36:1336-1341 (1988); Brandon et al., J. Agric. Food Chem. 39:327-335 (1991); and Brandon et al., Crop Sci. 32:1502-1505 (1992). The chKTI antibody binds the Kunitz soybean trypsin inhibitor (KTI). The chKTI antibody does not target GCC, and was used as an Ab-CDA conjugate control.

Mice were administered either a single intravenous injection of solution containing various doses of 5F9-CDA conjugate or control treatment once a week for three weeks (i.e., a fractionated regimen dosing at Days 0, 7, and 14), or a single acute dose of same (i.e., dosing only at Day 0). Tumor growth was monitored once per week for 11 weeks using vernier calipers. Mean tumor volume was calculated using the formula (V=[W²×L]/2). Anti-tumor efficacy of the experimental agents was determined by comparing the mean tumor volume of the vehicle control arm with each experimental agent.

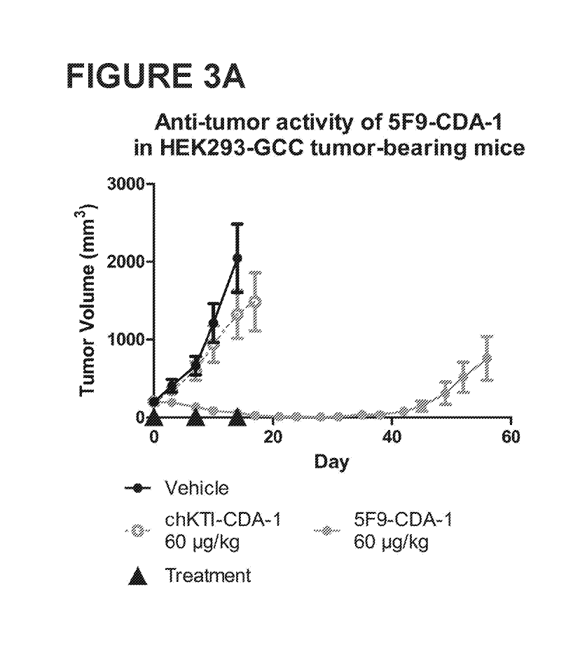

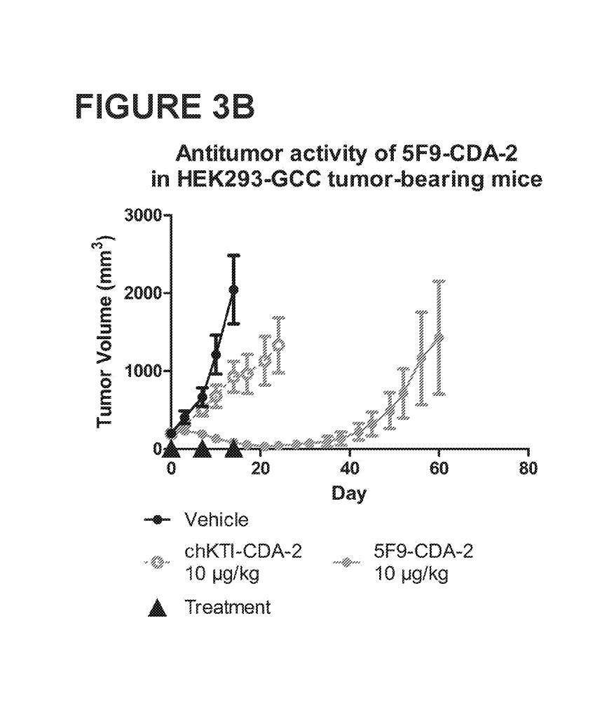

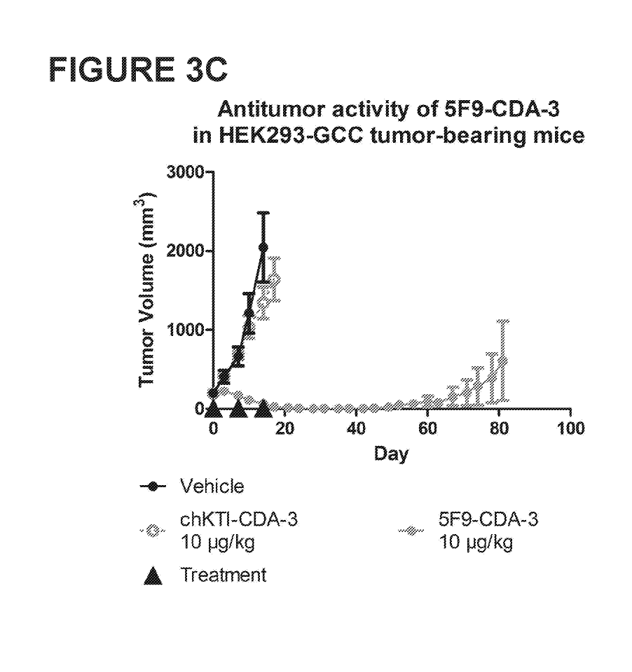

In mice bearing HEK293-GCC tumors, 5F9-CDA conjugates achieved durable anti-tumor activity (FIG. 3). Specifically, re-growth did not occur until 5-6 weeks following 5F9-CDA-1 and 5F9-CDA-2 treatment (FIGS. 3A and 3B). Anti-tumor activity was most pronounced in 5F9-CDA-3 studies, where tumor re-growth was typically not observed until 8-9 weeks post-treatment (FIG. 3C). Note that the group treated with 5F9-CDA-1 were dosed at 60 μg/kg whereas the groups treated with 5F9-CDA-2 and 5F9-CDA-3 were each dosed at 10 μg/kg, making the anti-tumor activity observed with 5F9-CDA-3 even more striking.

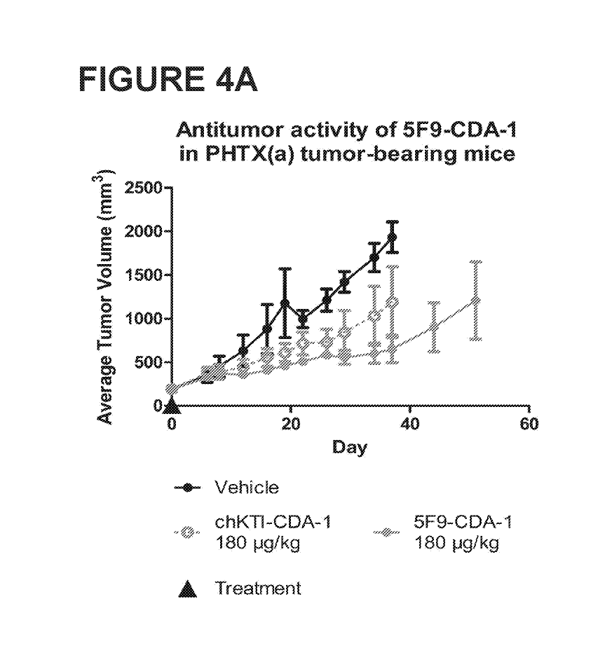

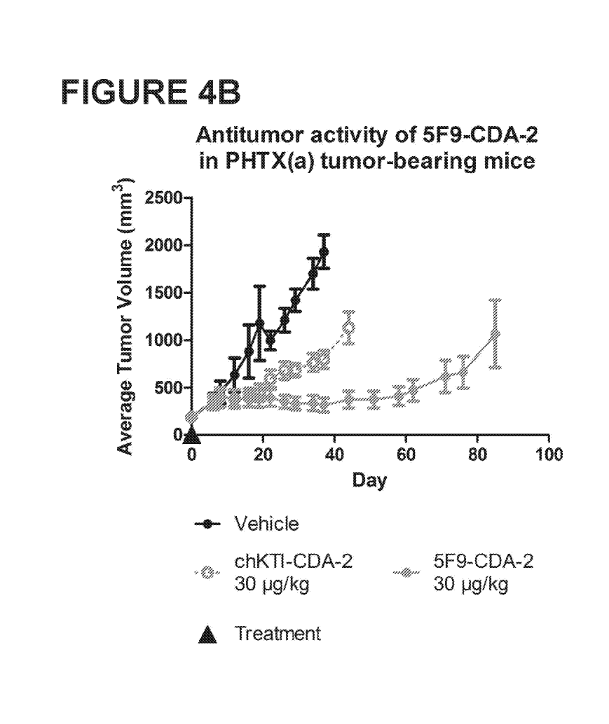

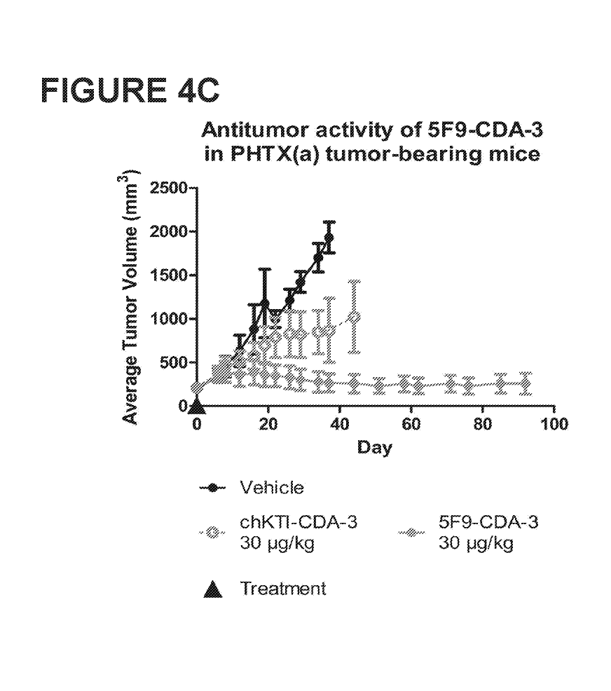

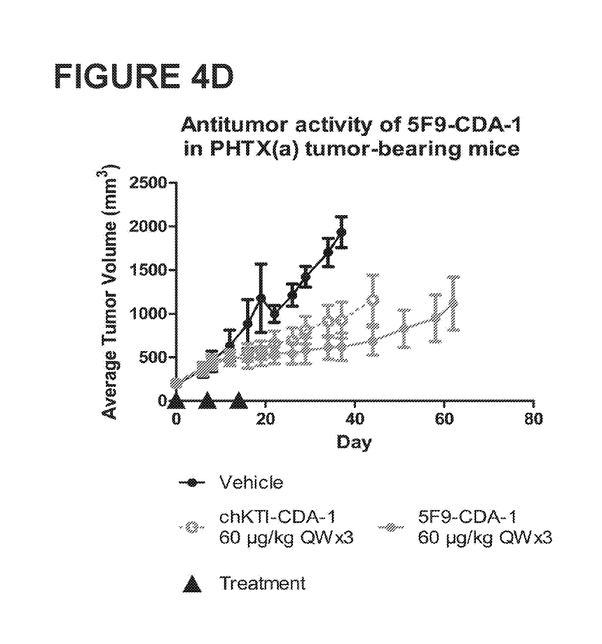

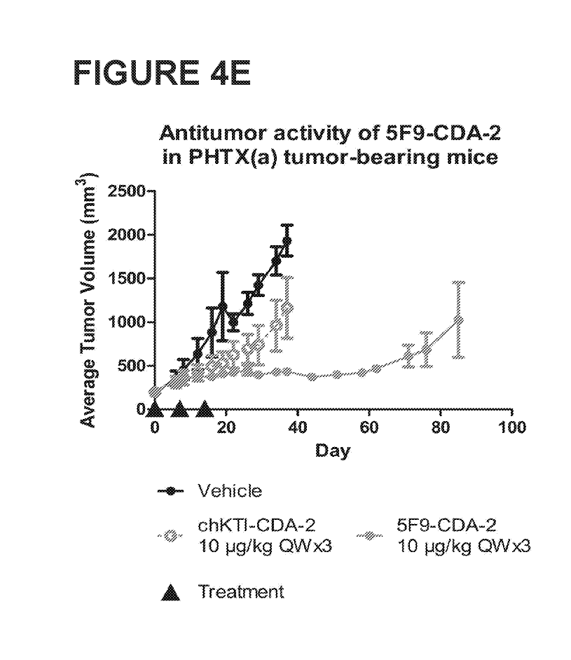

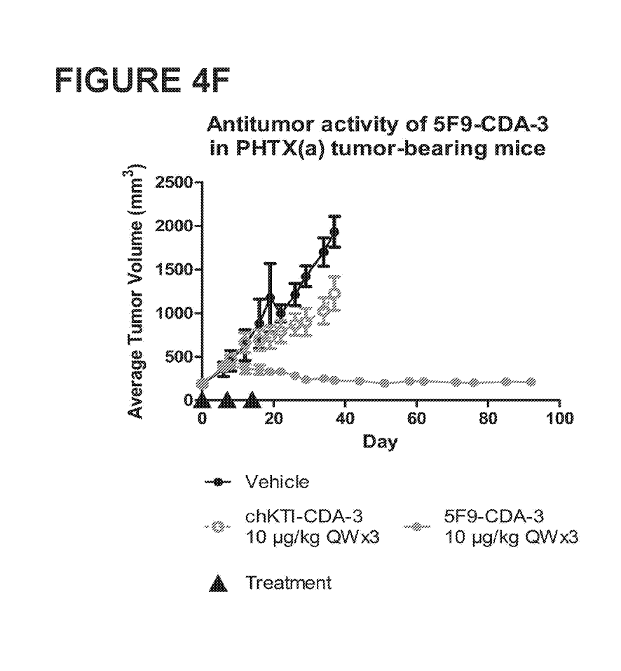

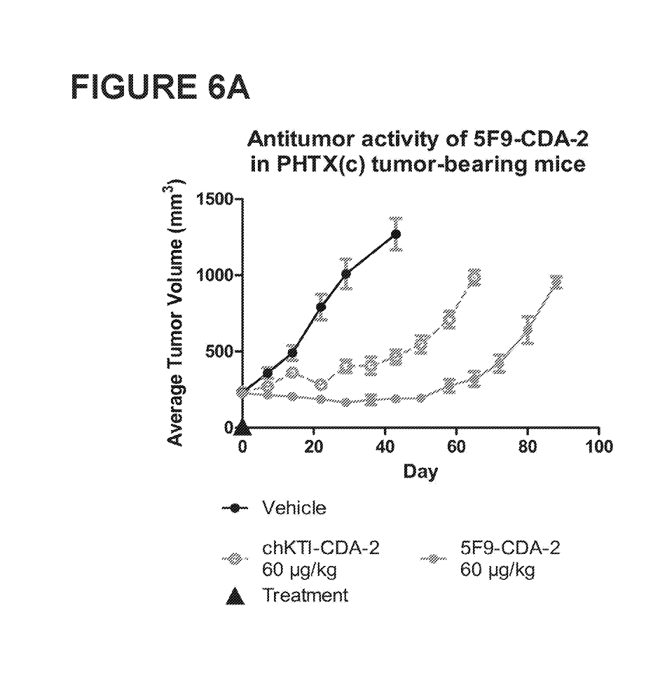

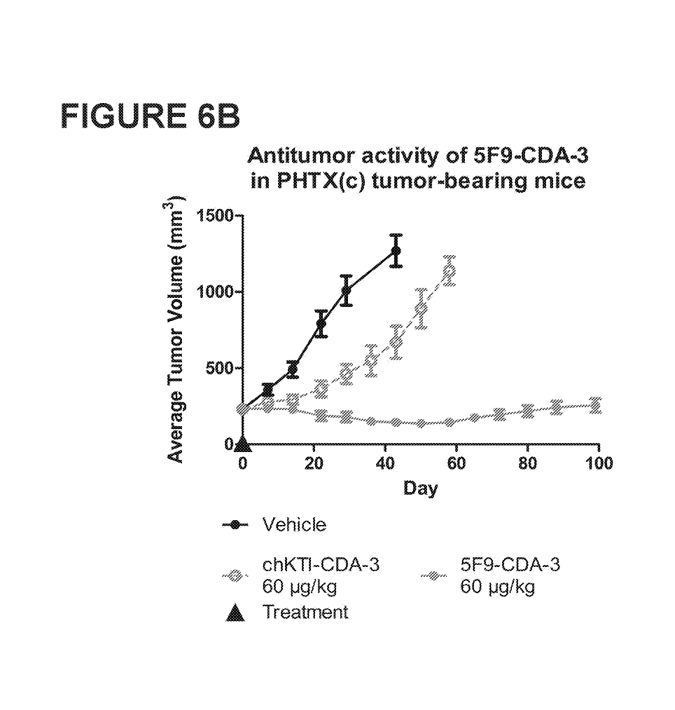

In primary human tumor xenograft (PHTX) colorectal models, treatment with 5F9-CDA-1 (60-180 μg/kg) delayed onset of tumor re-growth up to 5 weeks in PHTX(a) (FIGS. 4A and 4D), and PHTX(b) (FIG. 5A), which is a model that is refractory to MLN0264 (5F9-vcMMAE, see U.S. Pat. No. 8,785,600) treatment (FIGS. 4A and 4D). Growth inhibition was observed for even longer periods of time in PHTX(a), PHTX(b), and PHTX(c) tumors treated with 5F9-CDA-2 (FIGS. 4B, 4E, 5B, and 6A) or 5F9-CDA-3 (FIGS. 4C, 4F, 5C, and 6B) at lower doses (20-60 μg/kg). Similar to observations in HEK293-GCC tumor-bearing mice, intravenous administration of 5F9-CDA-3 yielded the longest delay of tumor re-growth, ranging from at least 8 to 14 weeks post-treatment.

Tumor/control (T/C) values are shown in Table 12 for in vivo efficacy studies performed in each primary tumor model. T/C is a metric that reports the tumor size for a given treatment arm (T) relative to the control arm (C). Strong anti-tumor activity is generally defined as a T/C≤0.40. For each study, T/C was calculated on the last day that the control arm was measured. 5F9-CDA-1 achieved a T/C value ≤0.40 at higher doses (90 and 120 μg/kg), whereas both 5F9-CDA-2 and 5F9-CDA-3 achieved T/C values ≤0.40 at lower doses (20-45 μg/kg) in each of the models.

TABLE 12

| | HEK293-GCC | PHTX(a) | PHTX(b) | PHTX(c) |
|---|---|---|---|---|
| | Day 14 | Day 37 | Day 47 | Day 43 |
| 5F9-CDA-1 120 μg/kg QW3 | | | 0.40 | |
| 5F9-CDA-1 180 μg/kg Single | | 0.34 | | |
| 5F9-CDA-1 60 μg/kg QW3 | 0.03 | 0.32 | | |
| 5F9-CDA-2 10 μg/kg QW3 | 0.04 | 0.22 | | |
| 5F9-CDA-2 30 μg/kg QW3 | | | 0.15 | |
| 5F9-CDA-2 30 μg/kg Single | | 0.16 | | |
| 5F9-CDA-2 60 μg/kg Single | | | | 0.15 |
| 5F9-CDA-3 10 μg/kg QW3 | 0.03 | 0.12 | | |
| 5F9-CDA-3 20 μg/kg QW3 | | | 0.09 | |
| 5F9-CDA-3 30 μg/kg Single | | 0.14 | | |
| 5F9-CDA-3 60 μg/kg Single | | | | 0.11 |

B. Pharmacokinetic/Pharmacodynamic Studies

Studies were performed to determine the pharmacokinetics (PK) of 5F9-CDA conjugates in HEK293-GCC tumor-bearing mice. PK studies followed the same subcutaneous inoculation protocol as the efficacy studies described above. When mean tumor volume reached approximately 500 mm³, animals were randomized into treatment groups.

Mice were administered a single intravenous dose of 5F9-CDA conjugates or vehicle at 30 μg/kg. Three animals were sacrificed at each defined time point (1, 24, 48, 96, 168, 336, and 504 hours) post-injection, and tumor and whole blood samples were harvested. The blood samples were transferred into serum separator tubes (BD Biosciences; Catalog No. 365956). Tumor tissues were formalin-fixed and paraffin-embedded for analysis of pharmacodynamic biomarker changes, as described below.

C. PK Assessment of Total Antibody and Total ADC in Plasma

Evaluation of the amount of total antibody and total antibody-drug conjugate (ADC) in murine blood samples following 5F9-CDA treatment was performed using a sandwich immunoassay. A 96-well plate was coated with protein comprising the extracellular domain of GCC, fused to a mouse Fc region. This portion of the GCC antigen is capable of capturing 5F9-CDA conjugate present in the samples. Ruthenylated donkey anti-human Fc-γ antibody was used to detect captured 5F9-CDA for the total antibody assay, while ruthenylated anti-CDA antibody was used to measure total ADC. In the presence of tripropylamine-containing read buffer, the ruthenium tag produces a chemiluminescent signal that is triggered by voltage. Chemiluminescence was measured on a MESO QuickPlex SQ 120 instrument (Meso Scale Diagnostics, Rockville, Md.).

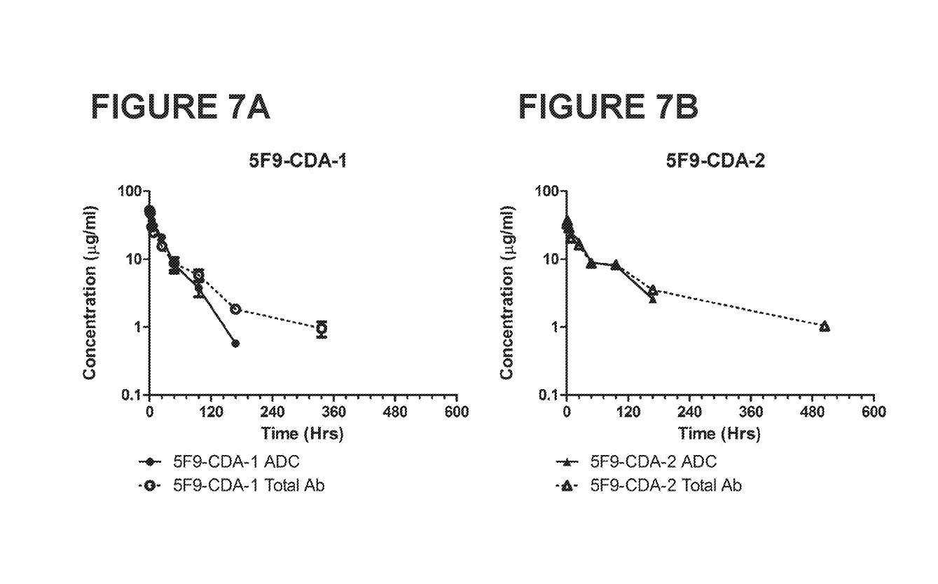

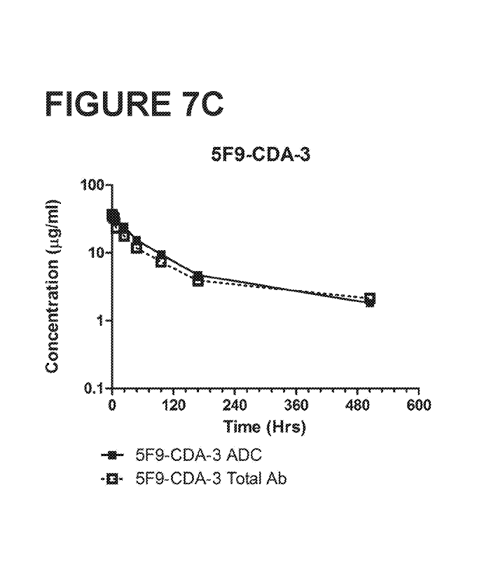

The total antibody and total ADC levels were appreciably different from one another following treatment with 5F9-CDA-1, particularly at the 168 and 336 hour time points (FIG. 7A). This difference suggests some degree of instability of the ADC in circulation. In contrast, total antibody and total ADC levels were comparable at all time points for both 5F9-CDA-2 and 5F9-CDA-3 (FIGS. 7B and 7C), indicating that these conjugates are stable in vivo.

Table 13 below reports PK parameters calculated using non-compartmental analysis. Notably, 5F9-CDA-3 demonstrated slower clearance (CL) than either 5F9-CDA-1 or 5F9-CDA-2. This difference resulted in greater exposure of the 5F9-CDA-3 conjugate over time, as reflected in the area under the curve (AUC) values.

TABLE 13

| | | t_{1/2} | C₀ | AUCₗₐₛₜ | AUC_{INF} | CL | Vss |
|---|---|---|---|---|---|---|---|
| Analyte | Treatment | (hr) | (μg/mL) | (hr * μg/mL) | (hr * μg/mL) | (mL/hr/kg) | (mL/kg) |
| Total Ab | 5F9-CDA-1 | 101.0 | 52.3 | 1,740 | 1,880 | 0.9 | 93.9 |
| Total ADC | | 30.6 | 45.7 | 1,510 | 1,540 | 1.1 | 43.0 |
| Total Ab | 5F9-CDA-2 | 150.3 | 33.2 | 2,300 | 2,526 | 0.81 | 149.46 |
| Total ADC | | 64.4 | 34.37 | 1,650 | 1,890 | 1.08 | 87.35 |
| Total Ab | 5F9-CDA-3 | 262.5 | 36.98 | 2,744 | 3,551 | 0.55 | 175.8 |
| Total ADC | | 190.2 | 38.32 | 3,261 | 3,763 | 0.52 | 116.22 |

D. PD Biomarker Activity Following Single Administration of 5F9 ADC

Pharmacodynamic (PD) biomarkers were detected by immunohistochemical staining of paraffin-embedded sections of HEK293-GCC#2 tumors. Sections were mounted onto glass slides, incubated with an EDTA-based solution (pH 9.0) for epitope retrieval for 20 min at 100° C., and blocked in serum-free protein block (Dako, Carpinteria, Calif.; Catalog No. X0909) to prevent non-specific antibody binding. Primary antibody solutions were then prepared with antibodies that recognize phospho-CHK1 (1:200; AbCam, Cambridge, Mass.; Catalog No. MIL2.091411.fzh) and phospho-γ-H2AX (1:1500; Cell Signaling Technologies, Beverly, Mass.; Catalog No. 9178), and incubated with the sections in a humidified chamber for 1 hour. Checkpoint kinase 1 (CHK1) is a serine/threonine-specific protein kinase whose activation is indicative of cell cycle arrest and certain forms of genotoxic stress, whereas γ-H2AX is a member of the histone family which becomes phosphorylated during the recruitment and localization of DNA repair proteins. DAB (3,3′-diaminobenzidine) polymer detection reagent was used for detection and visualization of the stains, and custom image analysis algorithms were used to determine the amount of staining relative to background staining. Results are reported as the percentage of antigen-positive cells/total viable cells in tissue sections.

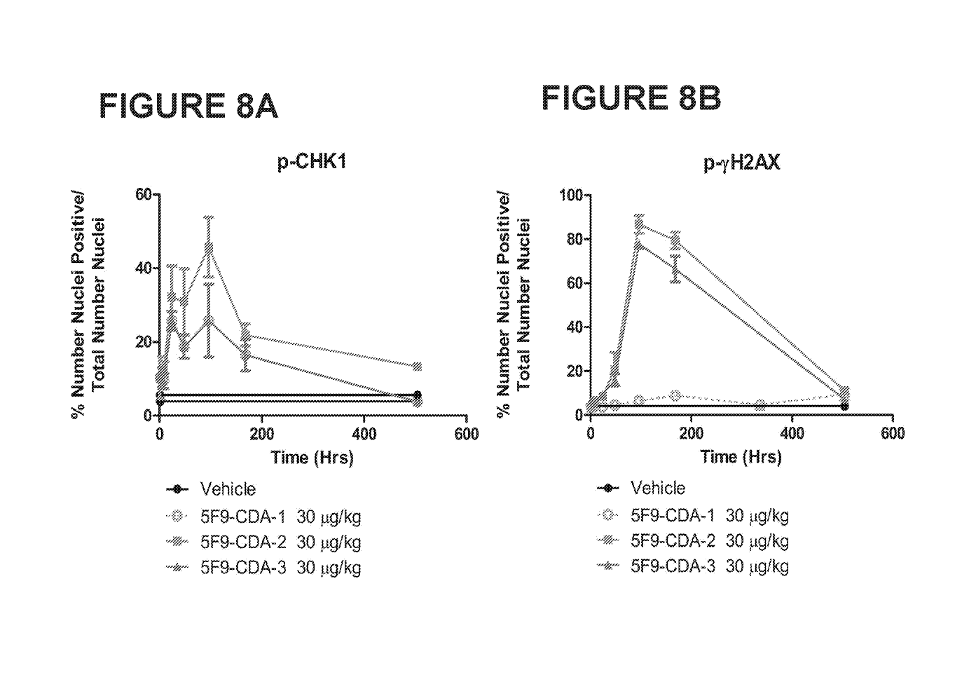

FIG. 8 shows that a single administration of each 5F9-CDA conjugate resulted in a marked increase in both phospho-CHK1 (FIG. 8A) and phospho-γ-H2AX (FIG. 8B), and that this increase was most pronounced following treatment with 5F9-CDA-2 and 5F9-CDA-3. Thus, DNA damage response biomarkers can be used to detect activity of 5F9-CDA conjugates in vivo.

In summary, 5F9-CDA-1, 5F9-CDA-2, and 5F9-CDA-3 have all been tested in vitro and in vivo for impact on cell/tumor growth in GCC positive models. Taken together, the data generated with each of these ADCs suggest that 5F9-CDA-3 possesses greater GCC-dependent activity in a broad range of models. While the margin of activity for 5F9-CDA-2 and 5F9-CDA-3 are comparable in vitro, the ADCs begin to separate when tested in vivo. The tolerability of each ADC following a single administration or repeat dosing is comparable in preclinical murine cancer models, yet antitumor activity is more pronounced compared to the corresponding isotype control ADC. Furthermore, the antitumor activity of 5F9-CDA-3 is consistently more durable than observed for 5F9-CDA-2. This is illustrated in FIGS. 3-6 using fractionated dosing and/or following a single administration. The PK data shown in FIG. 8 were calculated using non-compartmental analysis. Notably, 5F9-CDA-3 demonstrated slower clearance (CL) than either 5F9-CDA-1 or 5F9-CDA-2. This difference resulted in greater exposure of the 5F9-CDA-3 conjugate over time, as reflected in the area under the curve (AUC) values. Consistent with this observation, we have also observed most robust activation of the PD biomarkers pCHK-1 and pg-H2AX following a single administration of 5F9-CDA-3.

1-32. (canceled)

33. An antibody-drug conjugate or a pharmaceutically acceptable salt thereof, comprising: 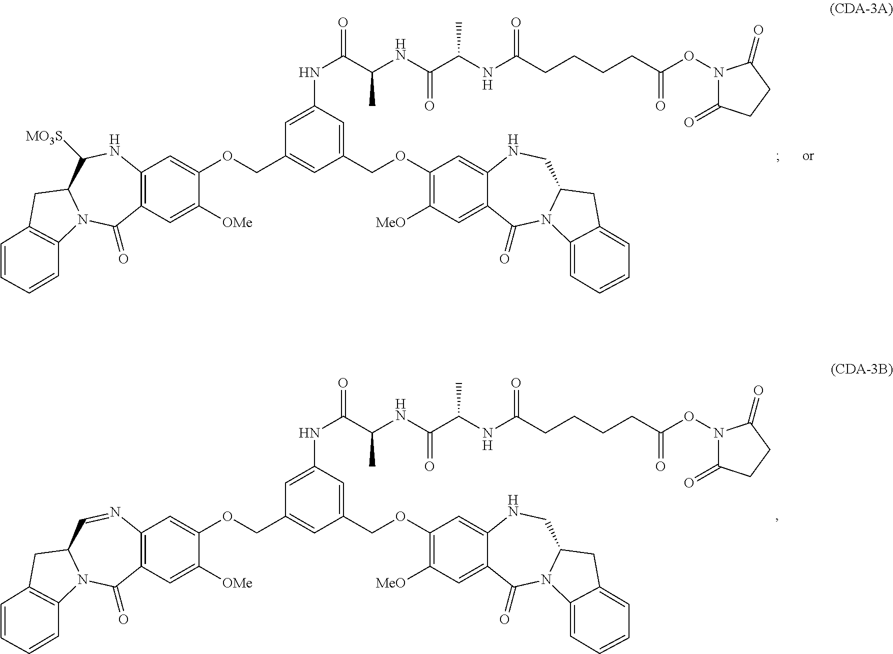
conjugated to an antibody, wherein the antibody comprises a heavy chain variable region (VH) comprising complementarity determining region (CDR) amino acid sequences of SEQ ID NO:1 (VHCDR1), SEQ ID NO:2 (VHCDR2), and SEQ ID NO:3 (VHCDR3); and a light chain variable region (VL) comprising complementarity determining region (CDR) amino acid sequences of SEQ ID NO:4 (VLCDR1), SEQ ID NO:5 (VLCDR2), and SEQ ID NO:6 (VLCDR3).

34. The antibody-drug conjugate or pharmaceutically acceptable salt of claim 33, comprising: [symbol]
wherein M is —H or a pharmaceutically acceptable cation; and wherein HN [symbol] is the antibody.

35. The antibody-drug conjugate or pharmaceutically acceptable salt of claim 33, wherein the antibody comprises a heavy chain variable region comprising an amino acid sequence of SEQ ID NO:7; and a light chain variable region comprising an amino acid sequence of SEQ ID NO:8.

36. The antibody-drug conjugate or pharmaceutically acceptable salt of claim 33, wherein the antibody comprises a heavy chain comprising an amino acid sequence of SEQ ID NO:9; and a light chain comprising an amino acid sequence of SEQ ID NO:10.

37. The antibody-drug conjugate or pharmaceutically acceptable salt of claim 33, wherein the drug:antibody ratio (DAR) ranges from about 1 to about 8.

38. The antibody-drug conjugate or pharmaceutically acceptable salt of claim 37, wherein the DAR ranges from about 2 to about 3.

39. A method of treating a subject for a cancer of gastrointestinal origin, comprising administering to the subject a therapeutically effective amount of the antibody-drug conjugate or pharmaceutically acceptable salt of claim 33.

40. The method of claim 39, wherein the cancer of gastrointestinal origin is selected from colon cancer, colorectal cancer, rectal cancer, gastroesophageal cancer, stomach cancer, and esophageal cancer.

41. The method of claim 40, wherein the colorectal cancer is selected from colorectal adenocarcinoma, colorectal leiomyosarcoma, colorectal lymphoma, colorectal melanoma, and a colorectal neuroendocrine tumor, or any metastases thereof; wherein the stomach cancer is selected from gastric adenocarcinoma, gastric lymphoma, and gastric sarcoma, or any metastases thereof; and/or wherein the esophageal cancer is selected from squamous cell carcinoma and adenocarcinoma of the esophagus, or any metastases thereof.

42. A method of treating a subject for pancreatic cancer, comprising administering to the subject a therapeutically effective amount of the antibody-drug conjugate or pharmaceutically acceptable salt of claim 33.

43. A method of reducing or inhibiting growth of a GCC-expressing tumor in a subject, comprising administering to the subject a therapeutically effective amount of the antibody-drug conjugate or pharmaceutically acceptable salt of claim 33.

44. A method of reducing the number or size of metastatic lesions and/or reducing tumor load in a subject suffering from a GCC-expressing cancer, comprising administering to the subject a therapeutically effective amount of the antibody-drug conjugate or pharmaceutically acceptable salt of claim 33.

45. A method of prolonging survival time and/or maintaining or improving the quality of life of a subject suffering from a GCC-expressing cancer, comprising administering to the subject a therapeutically effective amount of the antibody-drug conjugate or pharmaceutically acceptable salt of claim 33.

46. A pharmaceutical composition comprising the antibody-drug conjugate or pharmaceutically acceptable salt of claim 33, and a pharmaceutically acceptable carrier.

47. The pharmaceutical composition of claim 46, wherein the antibody-drug conjugate or pharmaceutically acceptable salt is formulated in 10 mM histidine, 50 mM sodium chloride, 8.5% sucrose, 0.01% Tween-20, 50 μM sodium bisulfite, pH 6.2; or wherein the antibody-drug conjugate or pharmaceutically acceptable salt is formulated in 50 mM histidine, 6.7% sucrose, 0.1% polysorbate-80, 50 μM sodium bisulfite, pH 5.5.

48. A method of preparing an antibody-drug conjugate or a pharmaceutically acceptable salt thereof, comprising reacting: (1) an antibody comprising a heavy chain variable region (VH) comprising complementarity determining region (CDR) amino acid sequences of SEQ ID NO:1 (VHCDR1), SEQ ID NO:2 (VHCDR2), and SEQ ID NO:3 (VHCDR3); and a light chain variable region (VL) comprising complementarity determining region (CDR) amino acid sequences of SEQ ID NO:4 (VLCDR1), SEQ ID NO:5 (VLCDR2), and SEQ ID NO:6 (VLCDR3); with (2) a cytotoxic drug agent selected from: 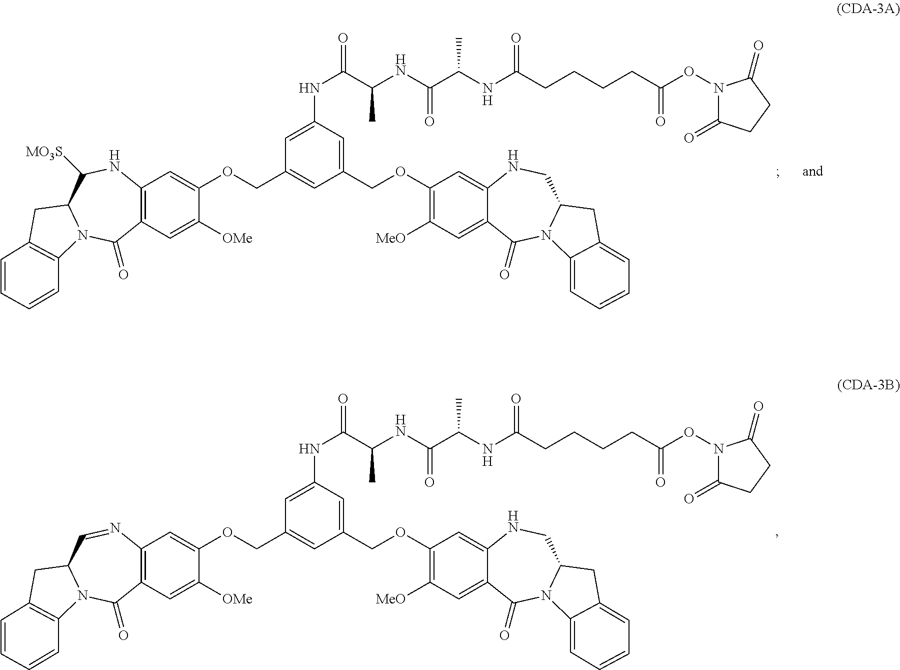
or pharmaceutically acceptable salts thereof.

49. The method of claim 48, wherein the reaction is carried out in a mixture of 75 mM EPPS buffer, pH 8.0, and dimethylacetamide; or wherein the reaction is carried out in a mixture of 130 mM EPPS buffer, pH 8.7, and dimethylacetamide.

50. The method of claim 49, wherein the amount of dimethylacetamide is 5-20% by volume.

51. The method of claim 48, wherein the reaction is carried out at a temperature of 22-25° C.

52. The method of claim 48, wherein the reaction is quenched with 150 mM histidine hydrochloride and 750 mM EPPS prior to purification; or wherein the reaction is quenched with 750 mM EPPS prior to purification.

53. The method of claim 48, wherein the method further comprises purifying the antibody-drug conjugate or pharmaceutically acceptable salt.

54. The method of claim 53, wherein the antibody-drug conjugate or pharmaceutically acceptable salt is purified using a chromatography column; or wherein the antibody-drug conjugate or pharmaceutically acceptable salt is purified using filtration followed by tangential flow filtration (TFF).


SEQUENCE LISTING (21 sequences)

SEQ ID NO: 1 - protein, 5 aa, Artificial Sequence
  source: /note=“Description of Artificial Sequence Synthetic peptide”
GYYWS

SEQ ID NO: 2 - protein, 16 aa, Artificial Sequence
  source: /note=“Description of Artificial Sequence Synthetic peptide”
EINHRGNTNDNPSLKS

SEQ ID NO: 3 - protein, 11 aa, Artificial Sequence
  source: /note=“Description of Artificial Sequence Synthetic peptide”
ERGYTYGNFDH

SEQ ID NO: 4 - protein, 11 aa, Artificial Sequence
  source: /note=“Description of Artificial Sequence Synthetic peptide”
RASQSVSRNLA

SEQ ID NO: 5 - protein, 7 aa, Artificial Sequence
  source: /note=“Description of Artificial Sequence Synthetic peptide”
GASTRAT

SEQ ID NO: 6 - protein, 9 aa, Artificial Sequence
  source: /note=“Description of Artificial Sequence Synthetic peptide”
QQYKTWPRT

SEQ ID NO: 7 - protein, 119 aa, Artificial Sequence
  source: /note=“Description of Artificial Sequence Synthetic polypeptide”
QVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 8 - protein, 107 aa, Artificial Sequence
  source: /note=“Description of Artificial Sequence Synthetic polypeptide”
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIK

SEQ ID NO: 9 - protein, 468 aa, Artificial Sequence
  source: /note=“Description of Artificial Sequence Synthetic polypeptide”
MGWSCIILFLVATATGVHSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 233 aa, Artificial Sequence
  source: /note=“Description of Artificial Sequence Synthetic polypeptide”
MGWSCIILFLVATATGVHSEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1444 bp, Artificial Sequence
  source: /note=“Description of Artificial Sequence Synthetic polynucleotide”
gaattcctcaccatgggatggagctgtatcatcctcttcttggtagcaacagctacaggtgtccactcccaggtgcagctacagcagtggggcgcaggactgttgaagccttcggagaccctgtccctcacctgcgctgtctttggtgggtctttcagtggttactactggagctggatccgccagcccccagggaaggggctggagtggattggggaaatcaatcatcgtggaaacaccaacgacaacccgtccctcaagagtcgagtcaccatatcagtagacacgtccaagaaccagttcgccctgaagctgagttctgtgaccgccgcggacacggctgtttattactgtgcgagagaacgtggatacacctatggtaactttgaccactggggccagggaaccctggtcaccgtcagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataatagggataacagggtaatactagag

SEQ ID NO: 12 - DNA, 722 bp, Artificial Sequence
  source: /note=“Description of Artificial Sequence Synthetic polynucleotide”
gcggccgcctcaccatgggatggagctgtatcatcctcttcttggtagcaacagctacaggtgtccactccgaaatagtgatgacgcagtctccagccaccctgtctgtgtctccaggggaaagagccaccctctcctgcagggccagtcagagtgttagcagaaacttagcctggtatcagcagaaacctggccaggctcccaggctcctcatctatggtgcatccaccagggccactggaatcccagccaggttcagtggcagtgggtctgggacagagttcactctcaccatcggcagcctgcagtctgaagattttgcagtttattactgtcagcagtataaaacctggcctcggacgttcggccaagggaccaacgtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgaccctgagcaaagcagactacgagaaacacaaagtctacgcctgcgaagtcacccatcagggcctgagctcgcccgtcacaaagagcttcaacaggggagagtgttagtctaga

SEQ ID NO: 13 - DNA, 112 bp, Artificial Sequence
  source: /note=“Description of Artificial Sequence Synthetic primer”
ataagaatgcggccgcctcaccatgggatggagctgtatcatcctcttcttggtagcaacagctacaggtgtccactccgaaatagtgatgacgcagtctccagccaccctg

SEQ ID NO: 14 - DNA, 45 bp, Artificial Sequence
  source: /note=“Description of Artificial Sequence Synthetic primer”
gccaccgtacgtttgatttccacgttggtcccttggccgaacgtc

SEQ ID NO: 15 - DNA, 103 bp, Artificial Sequence
  source: /note=“Description of Artificial Sequence Synthetic primer”
ccggaattcctcaccatgggatggagctgtatcatcctcttcttggtagcaacagctacaggtgtccactcccaggtgcagctacagcagtggggcgcaggac

SEQ ID NO: 16 - DNA, 45 bp, Artificial Sequence
  source: /note=“Description of Artificial Sequence Synthetic primer”
ggaggctgagctgacggtgaccagggttccctggccccagtggtc

SEQ ID NO: 17 - DNA, 1407 bp, Artificial Sequence
  source: /note=“Description of Artificial Sequence Synthetic polynucleotide”
atgggatggagctgtatcatcctcttcttggtagcaacagctacaggtgtccactcccaggtgcagctacagcagtggggcgcaggactgttgaagccttcggagaccctgtccctcacctgcgctgtctttggtgggtctttcagtggttactactggagctggatccgccagcccccagggaaggggctggagtggattggggaaatcaatcatcgtggaaacaccaacgacaacccgtccctcaagagtcgagtcaccatatcagtagacacgtccaagaaccagttcgccctgaagctgagttctgtgaccgccgcggacacggctgtttattactgtgcgagagaacgtggatacacctatggtaactttgaccactggggccagggaaccctggtcaccgtcagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 18 - DNA, 700 bp, Artificial Sequence
  source: /note=“Description of Artificial Sequence Synthetic polynucleotide”
atgggatggagctgtatcatcctcttcttggtagcaacagctacaggtgtccactccgaaatagtgatgacgcagtctccagccaccctgtctgtctccaggggaaagagccaccctctcctgcagggccagtcagagtgttagcagaaacttagcctggtatcagcagaaacctggccaggctcccaggctcctcatctatggtgcatccaccagggccactggaatcccagccaggttcagtggcagtgggtctgggacagagttcactctcaccatcggcagcctgcagtctgaagattttgcagtttattactgtcagcagtataaaacctggcctcggacgttcggccaagggaccaacgtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgaccctgagcaaagcagactacgagaaacacaaagtctacgcctgcgaagtcacccatcagggcctgagctcgcccgtcacaaagagcttcaacaggggagagtgttag

SEQ ID NO: 19 - protein, 4 aa, Artificial Sequence
  source: /note=“Description of Artificial Sequence Synthetic peptide”
ALAL

SEQ ID NO: 20 - protein, 4 aa, Artificial Sequence
  source: /note=“Description of Artificial Sequence Synthetic peptide”
GFLG

SEQ ID NO: 21 - protein, 4 aa, Artificial Sequence
  source: /note=“Description of Artificial Sequence Synthetic peptide”
ELLG